(19) United States
(12) Patent Application Publication
(10) Pub. No.: US 2024/0066060 A1
(43) Pub. Date: Feb. 29, 2024

(54) METHODS FOR TREATING INHERITED METABOLIC DISORDERS

(71) Applicant: BLUEROCK THERAPEUTICS LP, Cambridge, MA (US)

(72) Inventors: Ibrahim John DOMIAN, Swampscott, MA (US); Panagiotis DOUVARAS, Basking Ridge, NJ (US); Christoph PATSCH, Heidelberg (DE); Chew-Li SOH, Brooklyn, NY (US); Stephanie A. FISHER, Scarborough (CA); Maya SRINIVAS, Scarsdale, NY (US); Jing WANG, Syosset, NY (US); Carlos PALADINI, Hoboken, NJ (US); Ashley E. LEPACK, Wilton, CT (US)

(21) Appl. No.: 18/308,587

(22) Filed: Apr. 27, 2023

Related U.S. Application Data
(63) Continuation-in-part of application No. PCT/US2023/011916, filed on Jan. 31, 2023.
(60) Provisional application No. 63/305,624, filed on Feb. 1, 2022.
(60) Provisional application No. 63/313,420, filed on Feb. 24, 2022.
(60) Provisional application No. 63/314,960, filed on Feb. 28, 2022.
(60) Provisional application No. 63/348,405, filed on Jun. 2, 2022.
(60) Provisional application No. 63/352,014, filed on Jun. 14, 2022.
(60) Provisional application No. 63/431,487, filed on Dec. 9, 2022.

(51) Int. Cl. A61K 35/15; A61P 3/00; C12N 5/0787; G01N 33/68

(52) U.S. Cl. CPC A61K 35/15; A61P 3/00; C12N 5/0642; G01N 33/6893; G01N 2333/924; G01N 2400/40; G01N 2800/04; G01N 2800/52

(57) ABSTRACT

This disclosure provides compositions and methods for the treatment of inherited metabolic disorders in a subject. Provided herein are compositions and methods to treat Lysosomal Storage Diseases (LSD) in a subject using myeloid cells.

### CROSS REFERENCE TO RELATED APPLICATIONS

This application is a continuation-in-part of International Application No. PCT/US2023/011916, filed Jan. 31, 2023, which claims the benefits of priority from U.S. Provisional Application Nos. 63/305,624, filed Feb. 1, 2022; 63/313,420, filed Feb. 24, 2022; 63/314,960, filed Feb. 28, 2022; 63/348,405, filed Jun. 2, 2022; 63/352,014, filed Jun. 14, 2022 and 63/431,487, filed Dec. 9, 2022, the entire contents of which are incorporated herein by reference.

### INCORPORATION BY REFERENCE OF SEQUENCE LISTING

The sequence listing in the XML, named as 40178Z_SequenceListing.XML of 16 KB, created on Apr. 25, 2023, and submitted to the United States Patent and Trademark Office via Patent Center, is incorporated herein by reference.

### BACKGROUND

Lysosomes are specialized organelles that degrade and recycle macromolecules, connecting catabolic and anabolic metabolism. While all tissues require lysosomes to maintain homeostasis, the metabolically demanding brain is especially reliant on lysosome function as evident by the neurological phenotypes of many lysosomal storage diseases (LSDs). LSDs are inherited diseases that are characterized by the pathologic build-up of toxic material in the cells of the body because of a genetic defect resulting in enzyme deficiencies. There are upwards of 70 LSDs, typically occurring in infancy and childhood and together affect 1 in 5,000 live births worldwide. Many LSDs present as cognitive or motor disorders and affect many peripheral organs. LSDs often affect the central nervous system. Hurler Syndrome is a rare LSD (the most severe form of Mucopolysaccharidosis Type 1 (MPS1)), characterized by a spectrum of clinical phenotypes, including cognitive abnormalities and reduced life expectancy. Most LSDs are due to the genetic absence of a single catabolic enzyme which causes accumulation of the enzyme's substrate within lysosomes. In MPS1, deficiency in alpha-L-iduronidase (IDUA) causes accumulation of glycosaminoglycans (GAGs) leading to cellular damage and eventually cell death.

While enzyme replacement therapies using recombinant proteins, and bone marrow gene therapies delivering functional genes to hematopoietic stem cells are promising therapeutic candidates, they often fall short in delivering therapeutically relevant levels of enzyme to the central nervous system (CNS).

Accordingly, compositions and methods for treating lysosomal dysfunction in human LSDs are needed. Pluripotent stem cell-derived myeloid cells offer an exciting new therapeutic approach to the treatment of several LSDs. This cell+gene therapy contrasts with enzyme replacement and gene therapies where each therapy is tailored to the specific genetic defect.

### SUMMARY OF THE DISCLOSURE

In one aspect, this disclosure provides a method of treating a metabolic disorder comprising: administering pluripotent stem cell (PSC)-derived myeloid cells into a central nervous system of a subject to be treated, allowing the administered PSC-derived myeloid cells to engraft and to produce an enzyme, monitoring the level of the enzyme in a serum sample or a cerebrospinal fluid sample of the subject to be treated and monitoring the amount of substrate in the serum sample, a cerebrospinal fluid (CSF) sample or a urine sample of the subject to determine the progress of the treatment.

In some embodiments, said metabolic disorder is an inherited metabolic disorder.

In some embodiments, said inherited metabolic disorder is Hurler Syndrome, and said enzyme is alpha-L-iduronidase (IDUA).

In some embodiments, said inherited metabolic disorder is Sly Syndrome, and said enzyme is Beta-glucuronidase (GUSB).

In some embodiments, said central nervous system is consisting of spinal cord, brain, and cerebrospinal fluid (CSF).

In some embodiments, said subject is a mouse or human.

In some embodiments, said substrate is glycosaminoglycans

In some embodiments, said subject to be treated has an accumulation of undegraded substrate in brain cells and all other cells.

In some embodiments, said enzyme is used to reduce the accumulated undegraded substrate in the brain cells of the subject to be treated.

In some embodiments, a sustained reduction in total substrate levels greater than about 20% is indicative of successful treatment of said metabolic disorder.

In some embodiments, the amount of enzyme in said serum sample is compared to the amount of enzyme in said serum sample of a healthy subject.

In some embodiments, the amount of said cells to be injected is in the range of about 25×10⁶ to about 1250×10⁶ cells, about 50×10⁶ to about 1000×10⁶ cells, about 100×10⁶ to about 500×10⁶ cells, about 100×10⁶ to about 300×10⁶ cells and more preferably about 150×10⁶ to about 250×10⁶ cells.

### BRIEF DESCRIPTION OF THE FIGURES

The patent or application file contains at least 25 drawings executed in color. Copies of this patent or patent application publication with color drawing(s) will be provided by the Office upon request and payment of the necessary fee.

FIGS. 1A-1D. Overview of the Study Plan for the pilot, non-GMP, in vivo study to assess efficacy of myeloid cells transplantation in the IDUA-KO mouse model of Hurler Syndrome. A) Flow cytometry characterization of the myeloid cells that were transplanted into the brain of adult mice. B) Flow cytometry characterization of the myeloid cells that were transplanted into the brain of pediatric mice. C) Scheme depicting the two arms of the efficacy study including the time-points and the groups that were compared. D) Timeline of the in-vivo assessments and manipulations that took place during the “in-life” phase of the efficacy study.

FIGS. 2A-2B. The bar graphs depicted here show the biochemical analyses of CNS tissues from IDUA-KO mice 1 month after ICV delivery of myeloid cells. A) Considerable IDUA enzymatic activity can be detected in all CNS tissues of pediatric (at the time of transplantation) IDUA-KO animals assessed 1 month after myeloid cells transplantation, in a dose dependent manner. B) IDUA enzymatic activity in CNS tissues of adult (at the time of transplantation) IDUA-KO mice assessed 1 month after myeloid cell transplantation. Percentages show the enzymatic activity relative to that of WT littermates. Graph bars represent means, dots represent individual samples and error bars show standard deviation.

FIGS. 3A-3B. Quantification of the accumulated substrate of IDUA (represented as total GAGs) in CNS tissues of IDUA-KO animals 1 month after myeloid cell transplantation. The bar graphs depicted here show that myeloid cell transplantation into pediatric (A) or adult (B) IDUA-KO animals reduced the accumulation of the toxic substrate in all the CNS tissues and in a dose-dependent manner, compared to vehicle treated animals. Percentages show the reduction relative to the vehicle-treated animals. Graph bars represent means, dots represent individual samples and error bars show standard deviation.

FIGS. 4A-4B. The bar graphs depicted here show the biochemical analyses of CNS tissues from IDUA-KO mice 5 months after ICV delivery of myeloid cells. A) High IDUA enzymatic activity can be detected in all CNS tissues of pediatric (at the time of transplantation) IDUA-KO animals assessed 5 months after myeloid cell transplantation, in a dose dependent manner. B) Considerable IDUA enzymatic activity in CNS tissues of adult (at the time of transplantation) IDUA-KO mice assessed 5 months after myeloid cell transplantation. Percentages show the enzymatic activity relative to that of WT littermates. Graph bars represent means, dots represent individual samples and error bars show standard deviation.

FIGS. 5A-5D. A) Quantification of the accumulated substrate of IDUA (represented as total GAGs) in CNS tissues of IDUA-KO animals 1, 5 and 8 months after myeloid cell transplantation. Myeloid cell transplantation into pediatric (A) or adult (B) IDUA-KO animals show reduction of the accumulated toxic substrate in all CNS tissues and in a dose-dependent manner, compared to vehicle treated animals. The reduction is sustained between 1, 5 and 8 months after myeloid cell transplantation (compare with FIGS. 3A-3B). Percentages show the reduction relative to the vehicle-treated animals. Graph bars represent means, dots represent individual samples and error bars show standard deviation. B) Myeloid cells prevent GAG accumulation in the CNS of MPS I and MPS VII mouse models at 1-, 5- or 8-months post-treatment. Schematic depicting the experimental design of adult and juvenile myeloid cells transplantation in MPS I and MPS VII mouse models. Accumulation of GAGs at 1-, 5- and 8-months post-treatment in the brain, spinal cord, or CSF of the juvenile-treated MPS I cohorts. Myeloid cells were administered at a low (0.4×10⁶) or high dose (1.4×10⁶). Accumulation of GAGs at 1- and 5-months post-treatment in the brain, spinal cord, or CSF of the adult-treated MPS I cohorts. Accumulation of GAGs at 1-month post-treatment in the brain and spinal cord of the juvenile-treated MPS VII cohorts. Accumulation of GAGs at 1- and 5-months post-treatment in the brain and spinal cord of the adult-treated MPS VII cohorts. C) Enzymatic activity levels in CNS tissues of MPS I mice after myeloid cells treatment. Schematic depicting the experimental design of adult and juvenile myeloid cells transplantation in MPS I mouse model. IDUA enzymatic activity levels at 1-, 5-, or 8-months post-treatment in the brain, spinal cord, or CSF of the juvenile-treated MPS I cohort. IDUA enzymatic activity levels at 1- or 5-months post-treatment in the brain, spinal cord, or CSF of the adult-treated MPS I cohort. Myeloid cells were administered at a low (0.4×10⁶) or high dose (1.4×10⁶). IDUA levels are displayed as a percentage of wildtype animals from the same cohort and timepoint. D) Myeloid cells prevent GAG accumulation and restores deficient enzymatic activity in peripheral tissues of MPS I and MPS VII mice. Schematic depicting the experimental design of adult and juvenile myeloid cells transplantation in MPS I and MPS VII mouse models. Accumulation of GAGs in urine of MPS I mice sampled at regular intervals for the 1-, 5-, or 8-month cohort of the juvenile- or adult-treated MPS I mice. Accumulation of GAGs in urine of MPS VII mice sampled at regular intervals for the 1- or 5-month cohort of the juvenile- or adult-treated MPS I mice. IDUA enzymatic levels in serum of MPS I mice sampled at regular intervals for the 1-, 5-, or 8-month cohort of the juvenile- or adult-treated MPS I mice. IDUA enzymatic levels in serum of MPS VII mice sampled at regular intervals for the 1- or 5-month cohort of the juvenile- or adult-treated MPS I mice. GAG accumulation levels at 5-months post-treatment in the heart and liver of MPS I mice or the liver of adult-treated MPS VII mice.

FIGS. 6A-6B. The line graphs depicted here show the time-course of IDUA enzymatic activity and substrate quantification in the periphery of adult IDUA-KO animals 1 and 5 months after myeloid cell transplantation. A) IDUA activity can be detected in the peripheral blood of IDUA-KO animals that received high dose of myeloid cells via ICV injection. Consistent levels of IDUA activity can be detected at 1 and 5 months post myeloid cells transplantation. B) Reduction of total GAGs secreted in the urine can be detected for IDUA-KO animals that received a high or a low dose of myeloid cells via ICV injection. Sustained reduction in total GAGs was observed at 1 and 5 months post myeloid cell transplantation. The increase in GAG levels for the vehicle-treated group shows the progressive nature of the disease. Error bars show standard deviation.

FIGS. 7A-7B. The line graphs depicted here show the time-course of IDUA enzymatic activity and substrate quantification in the periphery of pediatric IDUA-KO animals 1 and 5 months after myeloid cell transplantation. A) IDUA activity can be detected in the peripheral blood of IDUA-KO animals after receiving a high or low dose of myeloid cells via ICV injection. Consistent levels of IDUA activity can be detected at 1 and 5 months post myeloid cell transplantation. B) Reduction of total GAGs secreted in the urine can be detected for IDUA-KO animals that received a high or a low dose of myeloid cells via ICV injection. Sustained reduction in total GAGs was observed between 1 and 5 months post myeloid cell transplantation. The increase in GAG levels for the vehicle-treated group shows the progressive nature of the disease. Error bars show standard deviation.

FIGS. 8A-8B. A) Hindlimb clasping, a behavioral test used as a marker of disease progression in models of neurodegeneration and cerebellar ataxia. The photos depict mice with different severity scores in the hindlimb clasping test. The table provides further explanation of the scoring. B) Photos depicting additional mice with severity scores (0-3) in the hindlimb clasping test with a WT and Knockout Mouse shown.

FIG. 9. Myeloid cells prevent ataxia defect in MPS I and MPS VII mice. Schematic depicting the experimental design of adult and juvenile myeloid cells transplantation in MPS I and MPS VII mouse models. Hindlimb clasping scores at 5- or 8-months post-treatment in juvenile MPS I mice. Hindlimb clasping scores at 1-, 2- and 5-months post-treatment in adult MPS VII mice. Hindlimb clasping scores at 1-month post-treatment in juvenile MPS VII mice.

FIGS. 10A-10B. A) Schematic denoting the experimental design of ICV transplantations in MPS I (IDUA-KO) and MPS VII (GUSB-KO) mouse models. B) Immunohistochemistry of two sagittal sections from adult IDUA-KO brain, 1 month after ICV delivery of Myeloid cells. Top photos are low magnification, stitched images from tiled scanned images of a lateral (left) or medial (right) section. Sections were stained for nuclei (DAPI; in grey), a myeloid marker (CD45; in red) and a human marker (hKu80, in blue). Yellow boxes depict the relative part of the section that is shown in higher magnification in the lower panels. Myeloid cells (arrows) are positive for all markers and are found in the choroid plexus and the meninges surrounding the brain.

FIG. 11. Immunohistochemistry of two sagittal sections from pediatric IDUA-KO brain, 1 month after ICV delivery of myeloid cells. Top photos are low magnification, stitched images from tiled scanned images of a lateral (left) or medial (right) section. Sections were stained for nuclei (DAPI; in grey), a myeloid marker (CD45; in red) and a human marker (hKu80, in blue) or for nuclei (DAPI; in grey), a microglia marker (IBA1; in red), a human marker (hKu80, in blue) and a myeloid marker (human specific CD163; in green). Yellow boxes depict the relative part of the section that is shown in higher magnification in the lower panels. Myeloid cells (arrows) are positive for all markers and are found in the choroid plexus and the meninges surrounding the brain.

FIG. 12. Immunohistochemistry of two sagittal sections from adult IDUA-KO brain, 5 months after ICV delivery of myeloid cells. Top photos are low magnification, stitched images from tiled scanned images of a lateral (left) or medial (right) section. Sections were stained for nuclei (DAPI; in grey), a myeloid marker (CD45; in red) and a human marker (hKu80, in blue). Yellow boxes depict the relative part of the section that is shown in higher magnification in the lower panels. Myeloid cells (arrows) are positive for all markers and are found in the choroid plexus and the meninges surrounding the brain. Some myeloid cells are entering the brain parenchyma showing typical microglial morphology.

FIGS. 13A-13G. A) Immunohistochemistry of a lateral sagittal section from pediatric IDUA-KO brain, 5 months after ICV delivery of myeloid cells. Photos on the left are low magnification, stitched images from tiled scanned images of a lateral section. Sections were stained for nuclei (DAPI; in grey), a microglia marker (IBA1; in red), a human marker (hNA, in blue) and a myeloid marker (human specific CD163; in green). Yellow boxes depict the relative part of the section that is shown in higher magnification in the lower panels. Myeloid cells (arrows) are positive for all markers and are found in the choroid plexus and the meninges surrounding the brain. B) Myeloid cells have long term engraftment in the MPS I mouse model. Representative pictures of MPS I brain sections 1-month post-treatment. C) Representative pictures of MPS I brain sections 8-months post-treatment showing myeloid cells. Pictures were developed using DAB-HRP due to autofluorescence in the tissue at this timepoint. D) Myeloid cells proliferative fraction decreases with time post-engraftment. Representative images of myeloid cells in MPS I mice 1-month post-treatment. Myeloid cells were checked for expression of the cell proliferation marker ki67. E) Quantification of the percentage of myeloid cells that are positive for ki67 at 1 and 5 months in the adult- and juvenile-treated cohorts of MPS I mice. F) Representative images of myeloid cells in MPS VII mice 1-month post-treatment. G) Quantification of the percentage of myeloid cells that are positive for ki67 at 1- and 5-months post-treatment in the adult-cohort of MPS VII mice.

FIGS. 14A-14B. Overview of the Study Plan for the pilot, non-GMP in vivo study to assess efficacy of myeloid cells transplantation in GUSB-KO mouse model of Sly Syndrome. A) Timeline of the “in-life” phase when various samples were collected from the mice and when hindlimb clasping was performed. B) Coronal section of the mouse brain atlas showing the site of myeloid cell delivery in both lateral ventricles (red arrows).

FIGS. 15A-15B. The bar graphs depicted here show the biochemical analyses of CNS tissues from GUSB-KO mice 1 month after ICV delivery of Myeloid cells. A) GUSB enzymatic activity in CNS tissues of adult GUSB-KO animals assessed 1 month after myeloid cell transplantation. B) Quantification of the accumulated substrate of GUSB (represented as total GAGs) in CNS tissues of GUSB-KO animals 1 month after myeloid cell transplantation. Myeloid cell transplantation into adult GUSB-KO animals reduced the accumulation of the toxic substrate in the CNS Percentages show the reduction of GAGs relative to the vehicle-treated animals. Graph bars represent means, dots represent individual samples.

FIGS. 16A-16B. A) & B) Hindlimb clasping test in adult GUSB-KO animals 1 and 5 months after myeloid cell transplantation. Myeloid cell transplantation reduced the behavioral deficit observed in GUSB-KO animals, especially 5 months after transplantation. Graph bars represent means, dots represent individual animals. Brain, Spinal Cord and Urine GAG levels are shown.

FIGS. 17A-17B. Immunohistochemistry of a lateral sagittal section from adult GUSB-KO brain, 1 month and 5 months after ICV delivery of myeloid cells. A) Photos on the left are low magnification, stitched images from tiled scanned images of a lateral section. Sections were stained for nuclei (DAPI in grey), a microglia marker (IBA1 in red), a human marker (hNA, in blue) and a myeloid marker (human specific CD163 in green). Yellow boxes depict the relative part of the section that is shown in higher magnification in the lower panels. Myeloid cells (arrows) are positive for all markers and are found in the choroid plexus and the meninges surrounding the brain. B) Photo on the left show cells injected into the ICV distribute throughout the brain and penetrate into the parenchyma at five months post transplantation. Photos on the right show cells successfully travel and penetrate through the length of the spinal cord.

FIGS. 18A-18B. IDUA enzymatic activity in brain lysates of IDUA-KO mice 5 months after transplantation of wild-type (WT) or IDUA overexpressing (OE) myeloid cells. A) Graph showing IDUA enzymatic activity in IDUA-KO brain lysates, expressed as percentages relative to the activity of wild-type animals. B) Quantification of IDUA enzymatic activity in brain lysates of IDUA-KO mice after transplantation with microglia that were WT, OE or KO for the IDUA gene. Transplantation of either WT or OE microglia results in considerable levels of IDUA in brain lysates of IDUA-KO animals. IDUA Het and IDUA WT are heterozygous or wild-type littermates to the IDUA-KO animals. Graph bars represent means, dots represent individual animals and error bars show standard deviation.

FIGS. 19A-19C. Reduction of the accumulated substrate of IDUA (represented as total GAGs) in brain lysates of IDUA-KO mice 5 months after transplantation with wild-type (WT) or IDUA over-expressing (OE) myeloid cells. A) Graph showing the reduction of GAG levels in IDUA-KO brain lysates, expressed as percentages relative to the GAG levels of the IDUA-KO animals. B) Quantification of GAG levels in brain lysates of IDUA-KO mice after transplantation with microglia that were WT, OE or KO for the IDUA gene. Transplantation of either WT or OE microglia results in considerable reduction of GAGs in brain lysates of IDUA-KO animals, in levels close to the IDUA heterozygous animals. IDUA Het and IDUA WT are heterozygous or wild-type littermates to the IDUA-KO animals. Graph bars represent means, dots represent individual animals and error bars show standard deviation. C) Myeloid cells prevent GAG accumulation in the CNS and ataxia defects in MPS I and MPS VII mouse models. Schematic depicting the experimental design of adult and juvenile myeloid cells transplantation in MPS I and MPS VII mouse models. Accumulation of GAGs at 5 months post-treatment in the brain, spinal cord or CSF of the adult- or juvenile-treated MPS I cohorts. Myeloid cells were administered at a low (0.4×10⁶) or high dose (1.4×10⁶). Correlation between CSF and brain GAG levels at 5 months post-treatment in the juvenile MPS I cohort. Accumulation of GAGs at 5 months in the brain or spinal cord of the adult MPS VII cohort. Hindlimb clasping scores at 5 months post-treatment in the juvenile MPS I and adult MPS VII cohorts.

FIGS. 20A-20C. A) In vitro correction of the accumulated toxic substrate in MG-KO after co-culture with myeloid cells. Knock-out microglia (MG-KO) are derived from human iPSC lines in which a lysosomal enzyme has been knocked-out. In this case MG-KO was used to model the mucopolysaccharidosis family of LSDs by knocking out IDUA, SGSH, NAGLU or GUSB. These lines are models of the lysosomal storage disorders MPS1 (Hurler Syndrome), MPS3A (SanFilippo A Syndrome), MPS3B (SanFilippo B Syndrome) and MPS7 (Sly Syndrome) respectively. Co-culture of the diseased cells with myeloid cells results in considerable reduction of intracellular GAGs for all the tested models of LSDs, providing evidence of functional delivery of active lysosomal enzymes from myeloid cells to neighboring diseased cells. B) Myeloid cells restore MPS enzymes to MG-KO cells and reduced glycoprotein accumulations to wildtype levels through a mannose-6-phospate dependent process. Enzymatic activity levels of IDUA and GUSB found in the supernatant of myeloid cells after 96 h in vitro. Schematic depicting the transwell-based system developed to test cross-correction of enzymes by myeloid cells to MG-KO cells. Accumulation of GAGs in cell lysates of WT control cells (myeloid cells), MG-KOs and MG-KOs co-cultured with myeloid cells for 9-10 days. Each graph denotes a MG-KO deficient in a different enzyme: IDUA, GUSB, SGSH, and NAGLU. Values are depicted as percentage of the GAG levels found in untreated MG-KOs at the end of the assay. Accumulation of GAGs in cell lysates of MG-KO co-cultured with myeloid cells in the presence and absence of a mannose-6-inhibitor. Values are depicted as percentage of the GAG levels found in untreated MG-KOs at the end of the assay. C) Co-culture with myeloid cells restores enzymatic levels in enzyme-deficient MG-KOs. Enzymatic activity of IDUA, GUSB, NAGLU in cell lysates of myeloid cells and corresponding MG-KOs alone or after co-culturing with myeloid cells. Quantification of immunoreactivity by western blot of SGSH in cell lysates of myeloid cells, SGSH-KOs, and SGSH-KOs co-cultured with myeloid cells.

FIG. 21. In vitro evidence for lysosomal enzyme transfer into MG-KO after co-culture with myeloid cells. Knock-out microglia (MG-KO) are derived from human iPSC lines in which a lysosomal enzyme has been knocked-out. In this case we used MG-KO to model MPS1 (Hurler Syndrome), MPS3B (SanFilippo B Syndrome) and MPS7 (Sly Syndrome) by knocking out IDUA, NAGLU or GUSB respectively. Co-culture of the diseased cells with myeloid cells results in variable, detectable intracellular levels of each of the missing lysosomal enzyme, providing evidence of functional delivery of lysosomal enzymes from myeloid cells to neighboring diseased cells.

FIG. 22. In vitro evidence for functional GAA lysosomal enzyme transfer into MG-KO model of Pompe Disease after co-culture with myeloid cells. To model the LSD called Pompe disease, GAA-KO microglia were derived from human iPSCs in which GAA, the gene responsible for the production of the lysosomal enzyme acid alpha-glucosidase, has been knocked-out. Co-culture of GAA-KO cells with myeloid cells results in higher levels of glucosidase and lower levels of glycogen—the accumulating substrate of glucosidase in Pompe Disease—compared to GAA-KO cells. These results provide evidence of functional delivery of active glucosidase from myeloid cells to neighboring diseased cells.

FIGS. 23A-23B. Cell engineering design to treat MPS, e.g., MPS1. A) CRISPR-Cas9 strategy for insertion of aEF1-IDUA overexpression construct into AAVS1 locus. IDUA-OE cells produce high levels of functional IDUA. B) CRISPR-Cas9 strategy for generation of IDUA knockout in human induced pluripotent stem cells (hiPSCs). IDUA-KO produce no detectable amount of IDUA.

FIGS. 24A-24F. Human induced pluripotent stem cells were used to derive authentic microglia based on a publicly available protocol. Schematic of the differentiation process for myeloid cells A) microglia cells express canonical microglia markers B), have high recovery and viability after thaw C), Representative phase contrast images of polarized myeloid cells 12 hours after the addition of pHrodo-conjugated E. coli particles. Upper left insets depict the detected fluorescence of pHrodo particles after phagocytosis. Quantification of representative phagocytosis assay. Graph depicts pHrodo fluorescence after addition to myeloid cells. Images were acquired every 30 m for a span of 24 h D) and pro-inflammatory cytokine (TNFα, CXCL10 and IL-6) expression levels after polarization of myeloid cells to M1-like state using LPS and IFNγ. Graphs denote mean and SEM E). This bioprocess robustly produced CD45+ microglia in many different cell lines F).

FIGS. 25A-25C. A transwell culture system was used to co-culture genetically modified microglia to assay for the ability of enzymatic rescue. This system comprises a regular tissue culture treated dish that can accommodate an insert, creating two compartments that are separated by a microporous membrane. A) IDUA-KO microglia cells were cocultured with myeloid cells genetically overexpressing IDUA (IDUA-OE) for 1 week, separated and assayed for enzymatic activity and substrate accumulation. B) When cultured alone, IDUA c.2 show high enzymatic activity/levels compared to IDUA-KO. However, IDUA-KO co-cultured for 7 days (IDUA c.2+IDUA-KO) showed an increase in intracellular enzymatic activity. Reduction in intracellular GAG concentrations was observed when IDUA-KO were co-cultured with IDUA c.2 (IDUA c.2+.IDUA-KO). C) When cultured alone, high GAG concentrations were observed for the KO Control.

FIG. 26 Immunoreactivity to human-specific CD45 in the brain of NSGQ/NSGS mice 3 months after p3 administration of myeloid cells.

FIG. 27. Immunoreactivity to human-specific CD45 and ku80 in the spinal cord of an NSGQ mouse 3 months after P3 administration of myeloid cells.

FIG. 28. Immunoreactivity to human-specific CD45 and ku80 in the brain parenchyma. Ramified myeloid cells engrafted in the parenchyma can be seen in the striatum and ventral midbrain, among many other areas.

FIG. 29. Immunoreactivity to human-specific TMEM119 and ku80 in the striatum of an NSGQ mouse 3 months after P3 administration of myeloid cells.

FIG. 30. Immunoreactivity to CD163, IBA1, and human Nuclear Antigen (hNA) in the brain of an NSGQ mouse 3 months after P3 administration of myeloid cells.

FIGS. 31A-31B. A) Detection of the number of CD45+/hku80+ cells found in mouse brain (automatic counts); B) Extrapolated counts of number of CD45+/hku80+ cells found in mouse brain per hemisphere.

FIGS. 32A-32D. Derivation and characterization of myeloid cells. (A) Total myeloid cells produced in a T-75 flask in 3 iPSC lines [N≥6]; (B) Viability of myeloid cells after thaw for each of the 3 iPSC lines [N≥6]; (C) Panel of representative images depicting immunoreactivity of myeloid cells to CD45, CD68, P2RY12, TREM2, IB1, TMEM119 and PU.1 after 2 days in culture; (D) Representative flow-cytometry plots for CD45/CD14 and CX3CR1/CD11B in post-thaw myeloid cells [N=3 for each iPSC line].

FIGS. 33A-33G. Transcriptomic analysis of myeloid cells before and after engraftment. (A) UMAP plots of the Bian et al. 2020 dataset of fetal hematopoietic (CD45+) cells depicting the origin, Carnegie stage and cell type grouping of all cells; (B) Same UMAP plot as (A) displaying expression level of representative markers for Mac 1-3 and Mac 4 groups; (C) PCA of myeloid cells, hiPSCs and Bian et al 2020 dataset. PC plots on the right display the identity score for each cell when compared to the Mac 1-3 or Mac 4 signature. Violin plots display the distribution of results for each sample; (D) Heatmap of differentially expressed markers in each cell group of Bian et al 2020 when compared to all other groups combined; (E) Schematic depicting the workflow for bulk RNAseq of myeloid cells vs engrafted myeloid cells; (F) Dot plot displaying the average expression and percentage of cells expressing Mac 4 and Mac 1-3 genes in each group; (G) Heatmap of unique genes shown in (F) displaying data from bulk RNAseq of engrafted myeloid cells. Green boxes highlight known microglial markers.

FIGS. 34A-34G. Pluripotency panel and karyotype analysis of hiPSC lines 6, 82 and 83. (A) Pluripotency flow cytometry results for hiPSC line 6. Blue overlays represent the experimental unstained control. (B) Phase contrast images from hiPSC line 6 24 hr post-thaw at 4× and 10× magnification. (C) Pluripotency flow cytometry results for hiPSC line 82. Blue overlays represent the experimental unstained control. (D) Phase contrast images from hiPSC line 82 24 hr and 48 hr post-thaw at 4× and 10× magnification. (E) Pluripotency flow cytometry results for hiPSC line 83. Blue overlays represent the experimental unstained control. (F) Phase contrast images from hiPSC line 83 24 hr and 48 hr post-thaw at 4× and 10× magnification. (G) Karyograms for hiPSC lines 6, 82 and 83 (G-banded cells analyzed, n=20).

### DETAILED DESCRIPTION OF THE DISCLOSURE

### Definitions

Unless defined otherwise, all technical and scientific terms used herein have the same meaning as commonly understood by one of ordinary skill in the art to which this disclosure belongs.

As used herein, the term “about” refers to an approximately +/−10% variation from a given value.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject. As used herein a “healthy subject” is characterized as disease free and/or free of any metabolic diseases.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like.

The term “pluripotent stem cells” or “PSCs,” as used herein, has its usual meaning in the art, i.e., self-replicating cells that have the ability to develop into endoderm, ectoderm, and mesoderm cells. In some embodiments PSCs are human PSCs (“hPSC”). PSCs include embryonic stem cells (ESCs), induced pluripotent stem cells and human induced pluripotent stem cells (“iPS cells” or “iPSCs” or “hiPSCs”). The terms ES cells and iPS cells have their usual meaning in the art.

Terms such as “treating” or “treatment” or “to treat,” as used herein, refer to therapeutic measures that cure, restore regenerative function, slow down, lessen symptoms of, and/or halt progression of a diagnosed pathologic disease or disorder. Thus, those in need of treatment include those already with the disorder. In certain embodiments, a subject is successfully “treated” for a disease or disorder if the subject shows, e.g., total, partial, permanent, or transient, alleviation or elimination of any symptom associated with the disease or disorder.

The term “myeloid cells” as used herein, refer to PSC-derived cells that are differentiated following a hemopoietic differentiation, express myeloid markers (including but not limited to: CD45, CD11b, CD33, CD14, CX3CR1), and are able to perform regular myeloid functions including but not limited to phagocytosis, respond to external stimuli, secretion of cytokines and polarization to pro-inflammatory or anti-inflammatory states. These myeloid cells will become the tissue-resident macrophages upon delivery to different organs in a live organism, e.g., they will become microglia when delivered into the brain of live animals, as they will express canonical microglia markers including but not limited to TMEM119, IBA1, CD163, CX3CR1, CD45, CD206.

The term “microglial progenitor cells” as used herein, refer to PSC-derived myeloid cells.

The term “microglia” as used herein, refer to PSC-derived myeloid cells.

The term “substrate” as used herein, refer to glycosaminoglycans or other undegraded compounds accumulated as a result of lysosomal storage diseases (LSD).

The term “central nervous system” as used herein, refers to the spinal cord, brain, and cerebrospinal fluid (CSF).

The term “KO (knock out)” as used herein, refers to a cell line or an organism that has been genetically engineered to lack one or more specific genes.

The term “WT (wild type)” as used herein, refers to a typical or most common form, appearance or strain existing in the wild; a normal, non-mutated version of a gene common in nature or an allele at each locus required to produce the wild-type phenotype.

The term “MG-WT” as used herein, refers to wild type myeloid cells.

The term “heterozygous” as used herein, refers to having inherited different forms of a particular gene from each parent. A heterozygous genotype stands in contrast to a homozygous genotype, where an individual inherits identical forms of a particular gene from each parent.

The term “metabolic disorders” or “inherited metabolic disorders” as used herein, refers to different types of medical conditions caused by genetic defects most commonly inherited from both parents that interfere with the body's metabolism.

The term “cells” as used herein, refers to the basic membrane-bound unit that contains the fundamental molecules of life and of which all living things are composed.

The term “somatic cells” as used herein, refers to any cell of the body except sperm and egg cells. Somatic cells are diploid, meaning that they contain two sets of chromosomes, one inherited from each parent. Somatic cells include brain cells. Brain cells include, but are not limited to neurons, oligodendrocytes, astrocytes, microglia, perivascular macrophages, meningial macrophages, endothelial cells, pericytes, ependymal cells and blood cells.

The term “xenotransplantation” as used herein refers to the transplantation, implantation or infusion into a recipient of cells (e.g., hiPSC-derived microglia) from a human source into another mammal, e.g., a mouse.

The term “ramified” as used herein refers to a microglial cell found at specific locations throughout the brain and spinal cord. The ramified microglia is in a “resting” state morphology.

The term “heatmap” as used herein refers to a two-dimensional table of numbers as shades of colors. Heatmap is used to depict gene expression and other multivariate data. The dense and intuitive display makes heatmaps well-suited for presentation of high-throughput data. Heat maps rely fundamentally on color encoding and on meaningful reordering of the rows and columns.

The term “gait analysis” as used herein refers to a critical tool to test mouse models. The gait analysis provides quantifiable behavioral data on how a given disease, injury, or drug impacts an animal's movement.

### Generation of Myeloid Cells from Pluripotent Stem Cells

Disclosed herein is a serum- and feeder-free protocol to differentiate PSCs (including iPSCs) towards the myeloid lineage. Various methods known in the art can be employed in connection with the present disclosure. Muffat et. al, Nat Med. 2016 November; 22(11): 1358-1367, Pandaya et. al, Nat Neurosci. 2017 May; 20(5): 753-759, Abud et. al, Neuron 2017 Apr. 19; 94(2):278-293, Douvaras et. al, Stem Cell Reports, Volume 8, Issue 6, P1516-1524, Jun. 6, 2017, Van Wilgenburg PLOS ONE, https://doi.org/10.1371/journal.pone.0071098—August 2013, Haenseler et. al, Stem Cell Reports 2017 Jun. 6; 8(6):1727-1742 and Takata et. al, Immunity 2017 Jul. 18; 47(1):183-198. The disclosures of these are incorporated herein by reference. Microglia cells arise from myeloid progenitors in the yolk sack during embryonic development. In an attempt to mimic the embryonic development of these myeloid cells, we generated primitive streak-like cells from PSCs, followed by hematopoietic and myelopoietic cocktails in serum-free media. This resulted in the appearance of myeloid cells in the supernatant fraction of the culture that expressed myeloid markers including but not limited to CD45, CD14, CX3CR1, CD33, CD11b. These myeloid cells can continue to be generated in the supernatant fraction of the culture for a considerable amount of time, often reaching 3 to 4 months. Myeloid cells are typically collected and frozen around 30 days after initiation of the differentiation protocol (the exact timing of collection is PSC-line dependent and should be experimentally defined based on the day of maximum yield). The yield is usually between 25 and 120 myeloid cells for every starting PSC, and their post-thaw viability is 85±10%.

In some embodiments the present disclosure provides methods for generating myeloid cells from pluripotent stem cells. In some such embodiments the pluripotent stem cells are from any mammalian species, but preferably from humans. In some embodiments the pluripotent stem cells are either induced pluripotent stem cells (“iPS cells” or “iPSCs”), or embryonic stem cells (“ES cells” or “ESCs”). Such methods involve a step where the pluripotent stem cells are cultured under conditions that induce myeloid differentiation, leading to the generation of CD 45+/CD14 +/CX3CR1+ myeloid cells. In some embodiments the differentiation medium comprises BMP4, GM-CSF, VEGF, SCF, IL3, TPO, M-CSF and FLT31. In some embodiments the medium further optionally comprises bFGF.

In some of the embodiments, the pluripotent stem cells are cultured under conditions that induce myeloid differentiation, leading to the generation of CX3CR1+ myeloid cells. In some of the embodiments, the pluripotent stem cells are cultured under conditions that induce myeloid differentiation, leading to the generation of CD45+ myeloid cells. In some embodiments the pluripotent stem cells are cultured or expanded in a bioreactor. In some embodiments the pluripotent stem cells are cultured in a cell factory under active gassing. By “active gassing” is meant exerting or applying a gas mixture pressure gradient in the cell factory or cell factories. Gas mixtures contemplated by the present disclosure include ratios of about 1% to about 20% CO₂ to about 80% to about 99% air, about 3% CO₂ to about 97% air and about 5% CO₂ to about 95% air.

In some of the embodiments of the present disclosure that involve culturing pluripotent stem cells under conditions that induce myeloid differentiation (leading to the generation of CD45+/CD14+/CX3CR1+ myeloid cells), a multi-step process is used in which the cells are cultured with different combinations of cytokines and tissue culture media at each stage. Steps in these multi-step processes may result in inducing differentiation of pluripotent stem cells into primitive hemangioblasts, and/or inducing differentiation of primitive hemangioblasts into myeloid progenitors.

In some embodiments, the methods provided herein for the generation of CD45+/CD14+/CX3CR1+ myeloid cells from pluripotent stem cells comprise performing one or more of the following steps: First, contacting a cell culture with a first composition comprising BMP4 in a culture medium, wherein when the cell culture is initially contacted with the first composition the cell culture comprises pluripotent stem cells. A small molecule able to activate the same pathway as BMP4 can be used; Second, contacting the cell culture with a second composition comprising one or more of SCF, and VEGF, and optionally bFGF, (for example each of SCF, and VEGF, with or without bFGF) in a hematopoietic cell medium; Third, contacting the cell culture with a third composition comprising one or more of SCF, IL-3, TPO, M-CSF, and FLT3 ligand (for example each of SCF, IL-3, TPO, M-CSF, and FLT3 ligand) in a hematopoietic cell medium; and fourth, contacting the cell culture with a fourth composition comprising one or more of M-CSF, FLT3 ligand, and GM-CSF (for example each of M-CSF, FLT3 ligand, and GM-CSF) in a hematopoietic cell medium. In some embodiments all of the above four steps are performed in order. In some of such embodiments the medium used for any of these four steps is a serum free medium. In some of such embodiments the medium used for any of these four steps is a chemically defined medium.

In the first of the above four steps, in some embodiments a tissue culture medium suitable for maintenance of stem cells is used, while in other embodiments a tissue culture medium suitable for differentiation of stem cells is used. In the last three of the above four steps, any suitable hematopoietic cell medium can be used.

In some embodiments, when carrying out the methods described above or elsewhere herein for the generation of myeloid cells from pluripotent stem cells, instead of discarding the tissue culture supernatant when performing media changes, the supernatant is centrifuged, and the cells present in the supernatant are recovered and added back to the cell cultures. This is advantageous because certain of the key cell types induced during the conversion of pluripotent stem cells to myeloid cells are found predominantly in the cell supernatants—as opposed to being in the layer of cells that adheres to the cell culture plates. Thus, in some embodiments, when media is changed cells present in the culture supernatant are recovered and added back to the cell cultures. In some embodiments, for media exchanges performed when the cells are in contact with the third composition or the fourth composition, cells present in the culture supernatant are recovered and added back to the cell cultures. In some embodiments the present disclosure provides myeloid cells or microglial progenitor cells, such as those produced by the methods described herein. In some embodiments the present disclosure provides a “substantially pure” populations of such cells.

### Treatment of Metabolic Disorders

Disclosed herein are methods to treat patients with LSDs using a cell+gene therapy approach. Myeloid cells are generated from pluripotent stem cells, derived from a healthy donor. PSC-derived myeloid cells are injected into the central nervous system of a patient to be treated. In one embodiment, myeloid cells are delivered systemically by intravenous (IV) administration. In another embodiment, myeloid cells are delivered to the Central Nervous System via intracerebroventricular (ICV) injection or directly into the cerebrospinal fluid (CSF) of the subject to be treated. In another embodiment, the myeloid cells are administered via intracerebroventricular (ICV) injection into the lateral ventricles of the brain, the site where the cerebrospinal fluid (CSF) is produced, and the CSF flow starts. The direct injection of myeloid cells into the cerebrospinal fluid (CSF) is used to bypass the blood-brain barrier. Alternatively, the disclosure contemplates intraparenchymal administration of myeloid cells. For intraparenchymal administration, the myeloid cells are delivered directly into the brain parenchyma, that includes but is not limited to the striatum, forebrain and hippocampus, as representative sites of parenchymal administrations. The amount of myeloid cells to be administered via any of the aforementioned routes is in the range of about 25×10⁶ to about 1250×10⁶ cells, about 50×10⁶ to about 1000×10⁶ cells, about 100×10⁶ to about 500×10⁶ cells, about 100×10⁶ to about 300×10⁶ cells and more preferably about 150×10⁶ to about 250×10⁶ cells. These specific delivery methods ensure engraftment of the injected microglial progenitor cells/myeloid cells and delivery of the enzyme of interest in the CNS.

The levels of enzyme production are stable for long periods of time as the PSC-derived myeloid cells become resident myeloid cells of the tissue, providing a steady and continuous supply of the missing enzyme. By “stable for long periods of time” is meant about 5 months to about 25 years, about 10 months to about 25 years, about 15 months to about 25 years, about 20 months to about 25 years, about 2 years to about 25 years, about 5 years to about 25 years and about 10 years to about 25 years. Thus, the missing enzyme is constantly and consistently secreted from the transplanted cells and is entering the host's diseased cells, correcting the pathologic accumulation of toxic substrates, such as glycosaminoglycans, in the case of Hurler Syndrome and Sly Syndrome, thereby, resulting in correction of the underlying pathophysiology in the CNS tissues. Some amount of the enzyme produced in the CNS by the engrafted PSC-derived myeloid cells also accesses the blood stream, delivering the therapeutic enzyme to the periphery as well, reducing the total amount of accumulated substrate, as evidenced by a reduction in total urine GAGs. After transplantation, the level of the enzyme is monitored at different time points in a serum sample, a urine sample or a cerebrospinal fluid sample of the patient. In one embodiment the level of the enzyme is monitored at 1 month, 6 months, 12 months and 24 months after transplantation and annually thereafter. The amount of substrate or glycosaminoglycans is monitored in the serum sample, a urine or a cerebrospinal fluid sample of the patient 1 month, 6 months, 12 months and 24 months after transplantation and annually thereafter to determine the progress of the treatment. A sustained reduction in total substrate levels greater than about 20%, about 30%, about 40%, about 50%, about 60%, about 70%, about 80% or about 90% is indicative of successful treatment of said metabolic disorder compared to pre-treatment levels of the individual patient. For Hurler Syndrome, the enzyme is alpha-L-iduronidase (IDUA). For Sly Syndrome, the enzyme is Beta-glucuronidase (GUSB).

In accordance with the present disclosure, treatment of metabolic diseases can be shown by measuring the level of enzyme or the level of substrate or by the level of both enzyme and substrate. In one embodiment, successful treatment of a Hurler Syndrome is shown when the level of IDUA increases in the serum, urine and/or cerebrospinal fluid (CSF) of the patient compared to the state before the onset of the treatment. Successful treatment of a Hurler Syndrome is also shown when the level of glycosaminoglycans (GAGs) decreases in conventional bodily fluids including the urine, blood or cerebrospinal fluid of the patient compared to the state before the onset of the treatment.

In another embodiment, successful treatment of a Sly Syndrome is shown when the level of GUSB increases in the serum and cerebrospinal fluid (CSF) of the patient compared to the state before the onset of the treatment. Successful treatment of a Sly Syndrome is also shown when the level of glycosaminoglycans (GAGs) decreases in conventional bodily fluids including the urine, blood or cerebrospinal fluid of the patient compared to the state before the onset of the treatment. Where the subject is pediatric, assessment of the efficacy of the treatment disclosed herein can be shown by improvement of gross motor function as measured by the Peabody Developmental Motor Scale or Gross Motor Function Measure 5 months, 6 months, 7 months, 8 months, 9 months, 10 months, 11 months, 12 months, 15 months, 2 years, 3 years, 4 years post-treatment. Appropriate clinical standard assessments are appreciated by the skilled practitioner and accessed where the subject is not pediatric without undue experimentation. These clinical assessments are applicable 5 months, 6 months, 7 months, 8 months, 9 months, 10 months, 11 months, 12 months, 15 months, 2 years, 3 years, 4 years post-treatment up to 25 years post-treatment.

In accordance with the present disclosure successful treatment of the metabolic diseases provided herein is recognized by the skilled practitioner by neurocognitive assessment and gross motor assessment measured over a period of time. Neurocognitive assessment of the treated subject is also recognized by halting or delaying the progression of neurocognitive decline using standard clinically approved neurocognition tests.

Pluripotent stem cell-derived myeloid cells offer an exciting new therapeutic approach to the treatment of several LSDs. This contrasts with enzyme replacement and gene therapies where each therapy is tailored to the specific genetic defect.

In another embodiment, the lysosomal storage diseases treatable by the methods of the present disclosure include but are not limited to: MPS I (Hurler Syndrome), MPS IIIA (Sanfilippo A), MPS IIIB (Sanfilippo B), MPS VII (Sly Syndrome), Krabbe disease, Pompe (glycogen storage disease type II), GM1-gangliosidosis, GM2-gangliosidosis (Sandhoff/Tay-Sachs), Metachromatic leukodystrophy, MPS II (Hunter), MPS IIIC (Sanfilippo C), MPS IIID (Sanfilippo D), MPS IVA (Morquio syndrome A), MPS IX, MPS VI (Maroteaux-Lamy), Niemann—Pick disease types A and B, Adrenoleukodystrophy (ALD), Niemann—Pick disease type C2, Niemann-Pick disease type C1, Sphingolipid-activator deficiency, Galactosialidosis, GM2-gangliosidosis, (GM2-activator deficiency), GM3-gangliosidosis, Fabry disease, Gaucher disease types II and III, α-Mannosidosis, β-Mannosidosis, Fucosidosis, Aspartylglucosaminuria, Schindler disease, Sialidosis Galactosialidosis, Mucolipidosis II (I-cell disease), mucolipidosis III, Danon disease, Salla disease, Mucolipidosis IV, Multiple sulphatase deficiency, MCD and Neuronal Ceroid Lipofuscinosis.

Persons of skill in the art will understand that an effective amount of the myeloid cells used in the methods of the disclosure can be determined by routine experimentation. Based on in vitro and in vivo mouse data on Hurler Syndrome, literature-based human data (Hurler Syndrome), scaling between mouse-man and considering the changes in brain—vs CSF-GAG values, a Systems-based dose prediction model framework was developed. The model was then utilized to estimate human equivalent dose of myeloid cells (number of myeloid cells) that would release IDUA to metabolize increased CSF-GAG levels from disease to healthy range (Table 1 and Table 2).

TABLE 1
The effect of varying CSF-GAG levels (brain surface
area scaling between mouse-man) on the dose needed
for reducing initial CSF-GAG by 50, 80 or 90%.

| Diseased | Dose for 50% | Dose for 80% | Dose for 90% | |
|---|---|---|---|---|
| CSF-GAG | reduction from | reduction from | reduction from | |
| μg/mL | diseased baseline | diseased baseline | diseased baseline | |
| 10 | 46 million | 77 million | 92.5 | million |
| 20 | 92 million | 150 million | 173 | million |
| 30 | 138 million | 222 million | 254 | million |

TABLE 2
The effect of varying CSF-GAG levels (brain volume
scaling between mouse-man) on the dose needed
for reducing initial CSF-GAG by 50, 80 or 90%.

| Diseased | Dose for 50% | Dose for 80% | Dose for 90% | |
|---|---|---|---|---|
| CSF-GAG | reduction from | reduction from | reduction from | |
| μg/mL | diseased baseline | diseased baseline | diseased baseline | |
| 10 | 42 million | 70 million | 83.5 | million |
| 20 | 83 million | 135 million | 156 | million |
| 30 | 124 million | 200 million | 230 | million |

The disclosure contemplates that e.g., the dose for 50% reduction from diseased baseline (10 μg/ml diseased CSF-GAG) would require about 46 million myeloid cells, the dose for 80% reduction would require about 77 million myeloid cells and the dose for 90% reduction would require about 92.5 million cells for brain surface area scaling between mouse-man.

The disclosure contemplates that e.g., the dose for 50% reduction from diseased baseline (10 μg/ml diseased CSF-GAG) would require about 42 million myeloid cells, the dose for 80% reduction would require about 70 million myeloid cells and the dose for 90% reduction would require about 83.5 million myeloid cells for brain volume scaling between mouse-man.

The myeloid cells can be administered alone or optionally along with pharmaceutically acceptable carriers and excipients, in preformulated dosages. The administration of an effective amount of the myeloid cells will result in an increase in the lysosomal enzymatic activity of the cells of a patient sufficient to improve the symptoms of the disease.

The present disclosure in another embodiment provides treatment of lysosomal storage diseases through co-culture of wild type myeloid cells with diseased cells.

### Xenotransplantation

Disclosed herein are methods for xenotransplantation of human myeloid cells, such as microglia cells using the NSG mouse model. The immunocompromised background of the NSG (NOD/SCID/IL2R gamma) mouse permits evaluation of allogeneic and xenogeneic cell transplantation experiments without daily administration of immunosuppressive drugs to prevent graft rejection. NSG mice have no mature T cells or B cells, lack functional natural killer (NK) cells and have reduced numbers of lymphocytes and myeloid dendritic cells. Human growth factors have been reported to support the survival and stable engraftment of human myeloid cells (including microglia). The NSG-SGM3 (Coughlan, 2016) and the NSG-Q (Svoboda, 2019) mouse strains were developed to provide the required human growth factors to support myeloid survival and engraftment.

NSG-SGM3 mice used the present disclosure were developed on the NSG background. They are genetically immunocompromised. NSG-SGM3 mice contain 3 human transgenes; human stem cell factor (SCF), human granulocyte/macrophage-colony, stimulating factor 2 (GM-CSF), and human interleukin (IL-3). Triple transgenic NSG-SGM3 mice constitutively produce adequate circulating levels of these myelosupportive human cytokines correcting for the lack of cross-species activity, when administering human cells, providing cell proliferation and survival signals over time, which provides a stable environment for engraftment of human myeloid cells such as microglia.

The NSG-Q mouse is a model widely used in microglia xenotransplantations as it is immunocompromised and has stable expression of key myelosupportive human cytokines (CSF1, CSF2, KITLG, and IL3) relevant for survival of microglia and other myeloid cells (Svoboda, 2019).

The present disclosure is further illustrated by the following non-limiting examples.

### EXAMPLES

### Example 1

### Generation of Knock-Out Cell Lines

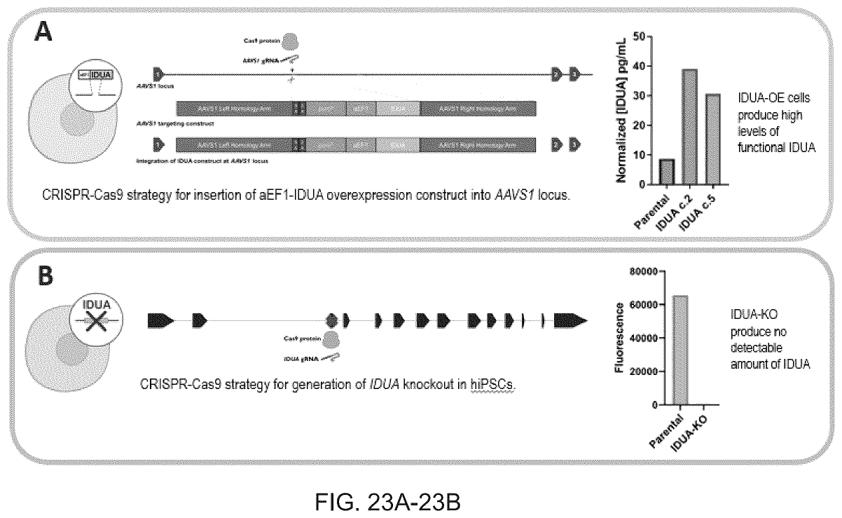

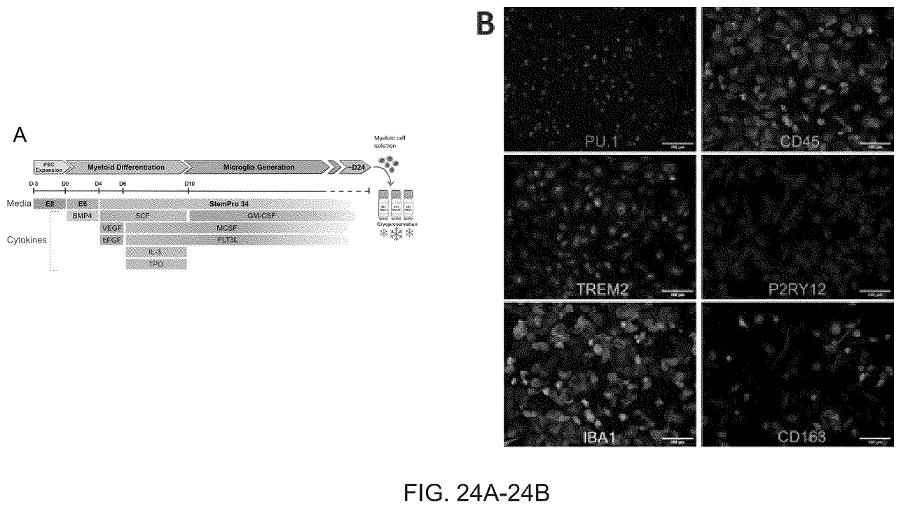

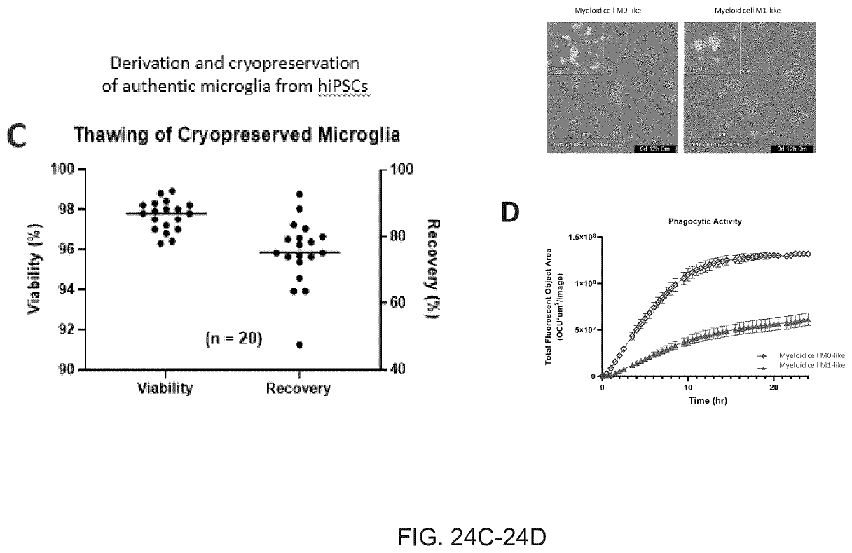

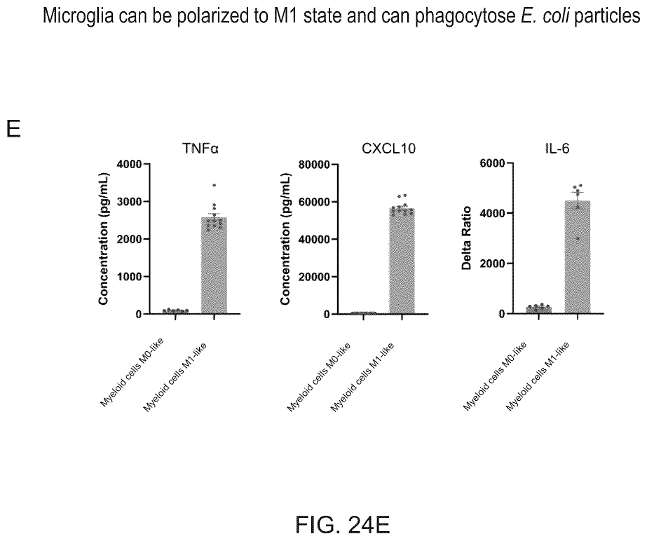

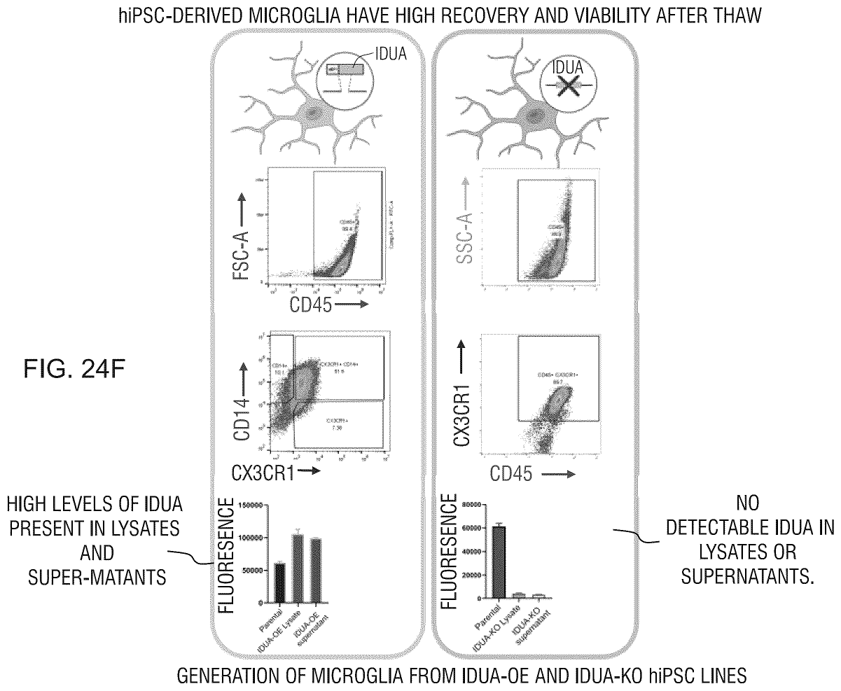

10 LSD-related genes were targeted to generate knock-out cell lines (null KOs) via CRISPR/Cas9-based editing: These genes are IDUA, GUSB, SGSH, NAGLU, GAA, GALC, GLB1, ARSA, HEXA and HEXB. These clones have been confirmed to be a null KO by genotype through sequencing, and null protein KO through either ELISA or Western Blot and enzyme activity assays. CRISPR/Cas9 enabled the creation of iPSC clones from a single parental line: a knock-in of an overexpression construct of IDUA enzyme for payload delivery and a knock-out of IDUA to use as target for enzyme cross-correction and as a negative control in animal studies. The genotypes were verified by Sanger sequencing and protein expression was confirmed by ELISA, resulting in minimal expression of the targeted knockout or increased expression in the overexpressing line. (FIGS. 23A-23B).

### Example 2

### In Vitro Cross-Correction Assay

Wildtype microglial cells (MG) were cultured on the bottom of a 6-well and knockout cells (PSC or MG) were co-cultured in a transwell for ˜2 weeks. Cells were either harvested or lysed in-well with Lysis buffer (1% Triton-X in HBSS, 1×Halt Protease Inhibitor). The total protein of each lysate is measured by Pierce BCA Protein Assay Kit (Thermo Fisher). Natural substrate accumulation is measured for each sample (GAG or Glycogen assay) and if possible, enzyme activity is also measured for each sample.

The results of these transwell cultures (in case of Hurler Syndrome) show that healthy microglia release IDUA enzyme into the culture media, the released IDUA is taken-up by IDUA-KO microglia and this leads to a reduction of the toxic accumulation of glycosaminoglycans (GAG) in the recipient IDUA knockout microglia. These results demonstrate that wildtype donor microglia can cross-correct the enzymatic deficiency in defective cells found in LSDs.

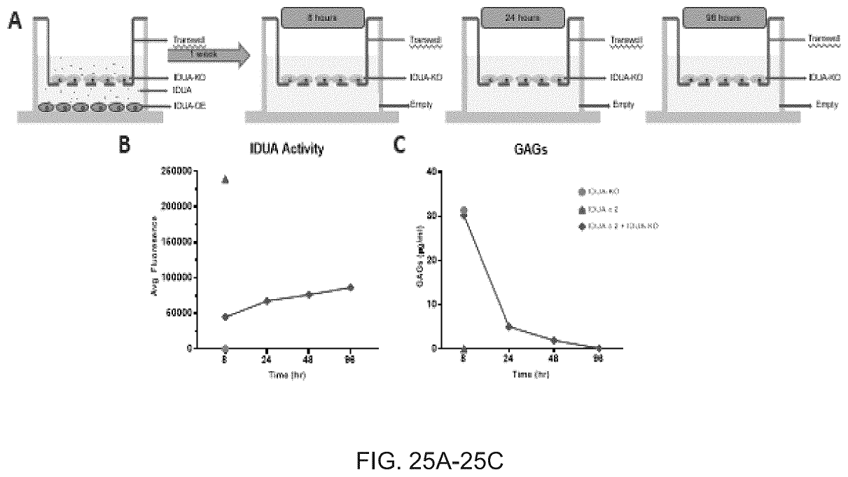

IDUA-OE and IDUA-KO hiPSC lines differentiated robustly into microglia. To demonstrate the ability of overexpressing cells to functionally correct diseased cells lacking a single lysosomal enzyme, IDUA-KO microglia were co-cultured with IDUA-OE microglia in a transwell system. An increase in intracellular IDUA enzymatic activity in IDUA-KO microglia was observed when co-cultured with the overexpressing line and a corresponding reduction in accumulation of GAG substrate. The enzymatic activity and total GAG accumulation results demonstrate that over expressing IDUA can metabolically cross-correct and restore function, by providing the appropriate missing enzyme. (FIGS. 25A-25C). Additional experimental data show that this general mechanism of cross-correction is true for several other LSDs, thus identifying the mechanism as an ideal cell therapy for LSDs.

### Example 3

### In Vivo Efficacy Assay

IDUA (alpha-L-iduronidase) knockout mice for Hurler Syndrome and GUSB (Beta-glucuronidase) knockout mice for Sly Syndrome were purchased from Jackson Labs.

### Hurler Syndrome

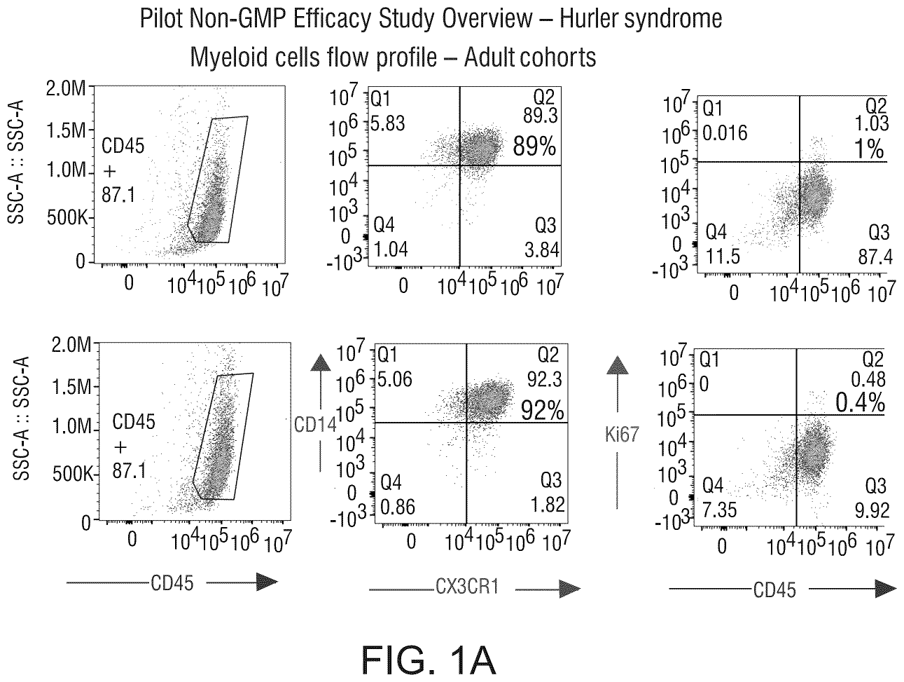

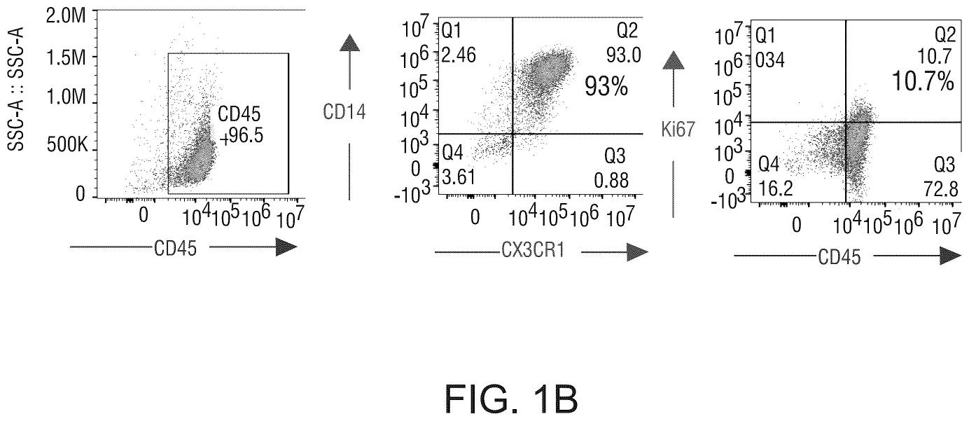

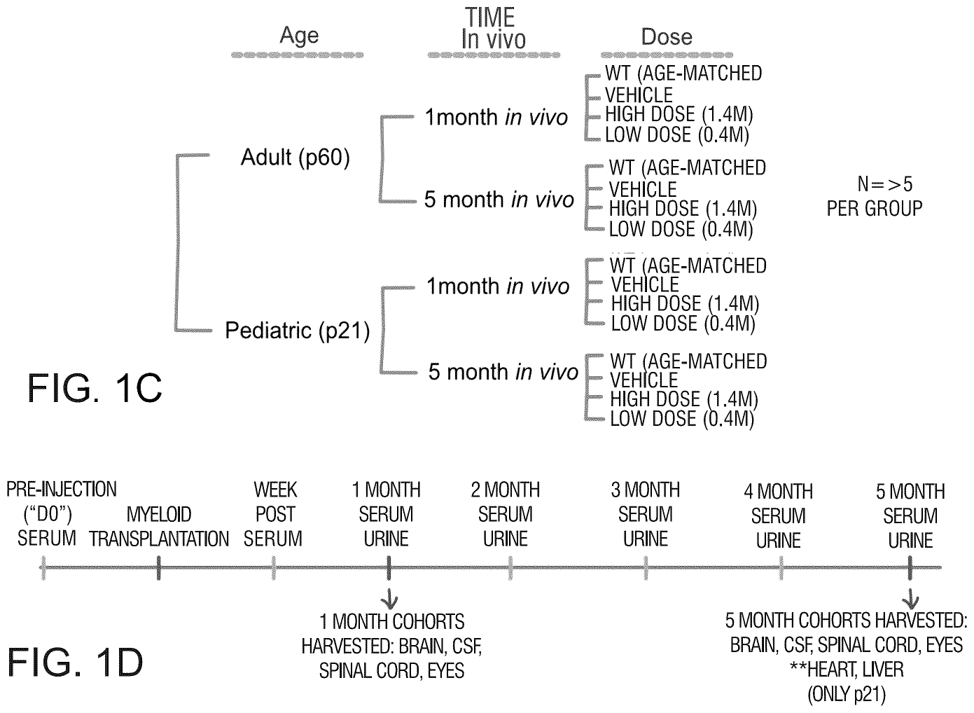

Microglial cells were transplanted into the brain of pediatric and adult IDUA knockout mice. 1.4×10⁶ cells are used for high dose and 0.4×10⁶ cells are used for low dose. At different time points (one month and five months), body fluids (serum, CSF, urine) and tissues (brain, spinal cord) are collected and the amount of IDUA enzyme and GAGs (glycosaminoglycans) are tested. FIGS. 1A-1D show the schematic overview of the non-GMP, in vivo study plan to assess the efficacy of myeloid cell transplantation in the IDUA-KO mouse model of Hurler Syndrome.

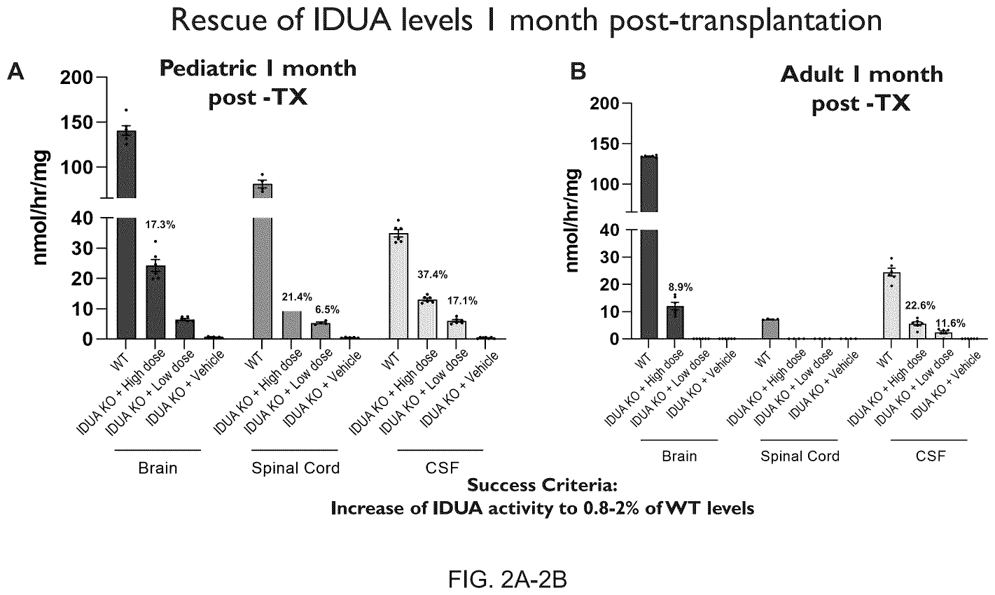

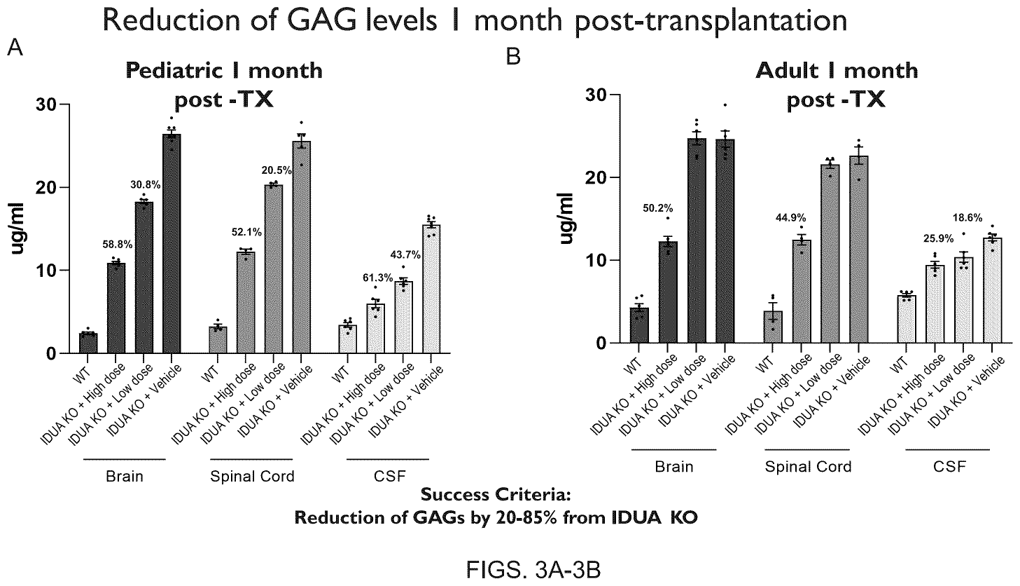

FIGS. 2A-2B show the biochemical analyses of CNS tissues from IDUA-KO mice 1 month after ICV delivery of myeloid cells. The IDUA enzyme level was rescued 1-month post-transplantation in pediatric and adult IDUA KO mice in a dose dependent manner. IDUA activity was increased to 10-15% of WT levels. FIGS. 3A-3B shows the quantification of the accumulated substrate of IDUA (represented as total GAGs) in CNS tissues of IDUA-KO animals 1 month after Myeloid cells transplantation. Myeloid cells transplantation into pediatric or adult IDUA-KO animals reduced the accumulation of the toxic substrate in all the CNS tissues in a dose-dependent manner compared to the vehicle treated animals. GAGs were reduced by 20-85% from IDUA KO mice relative to the vehicle-treated animals.

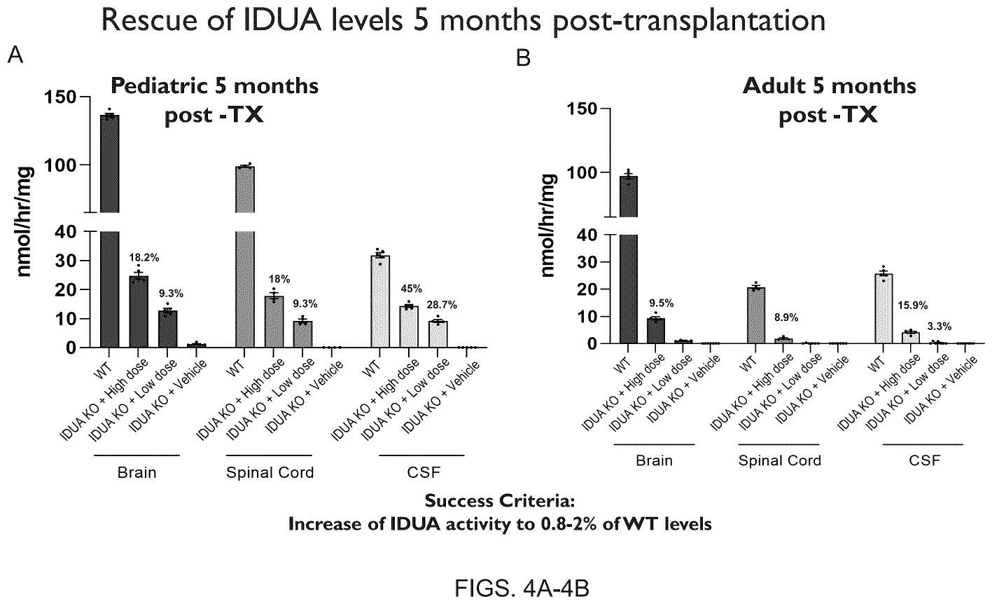

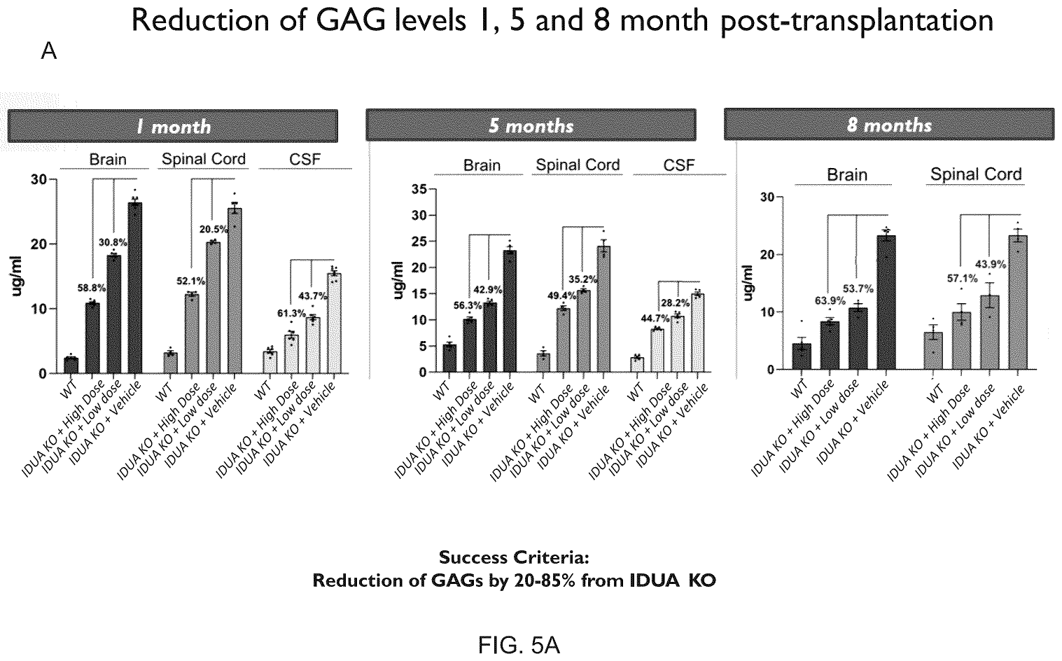

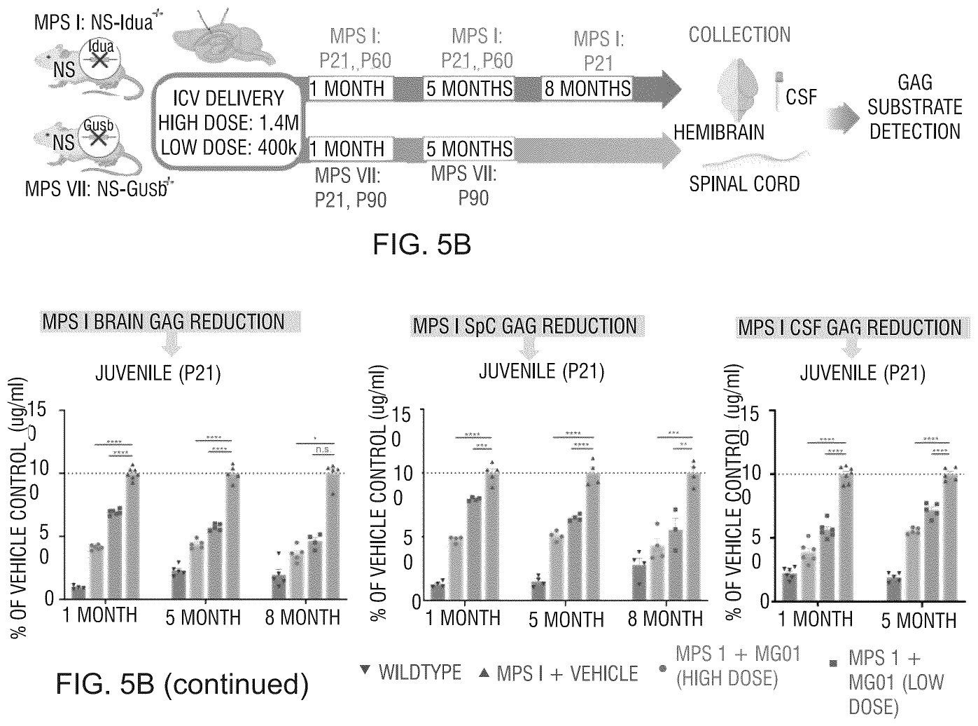

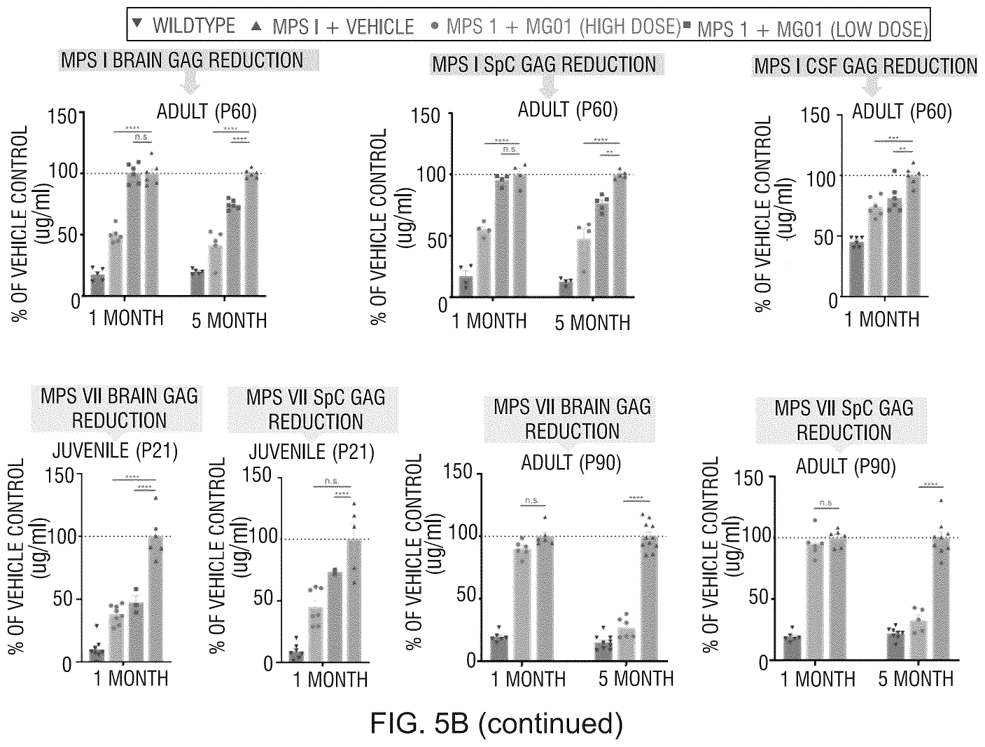

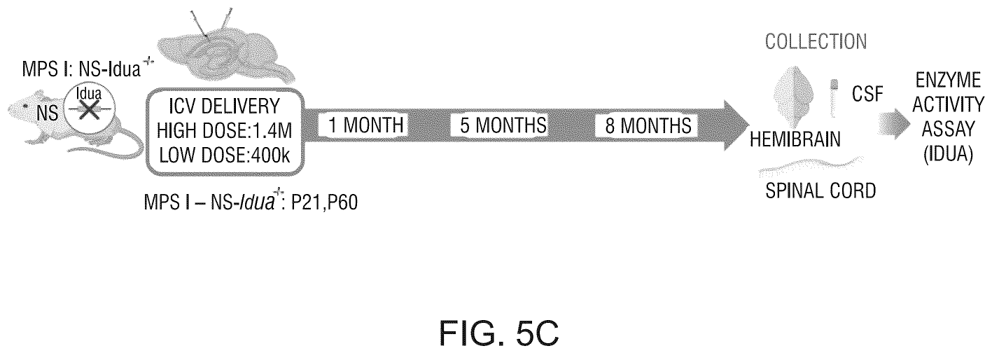

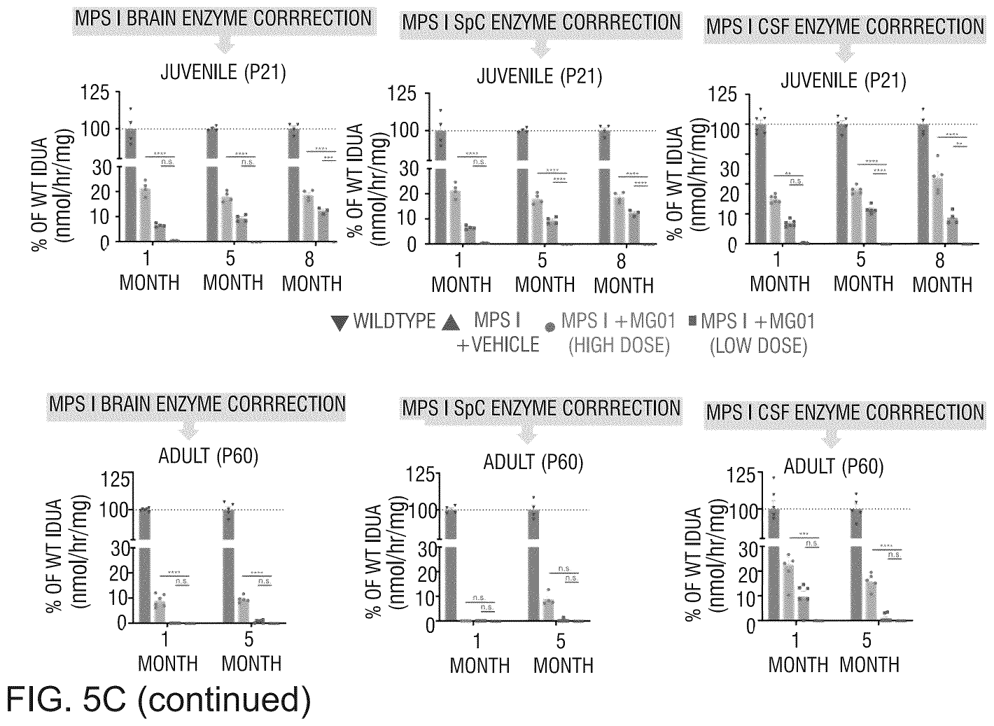

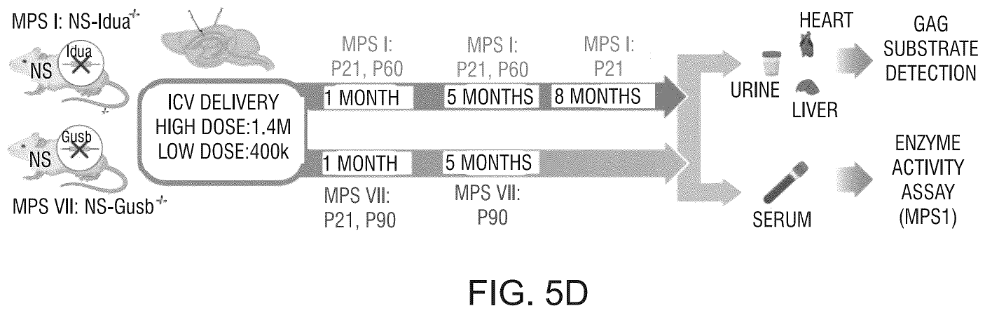

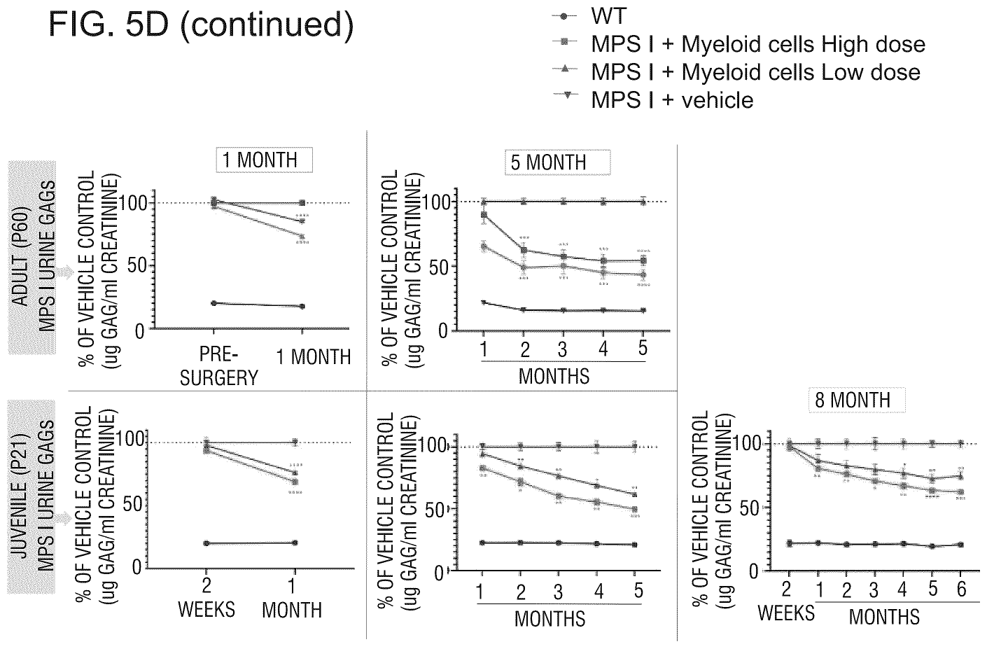

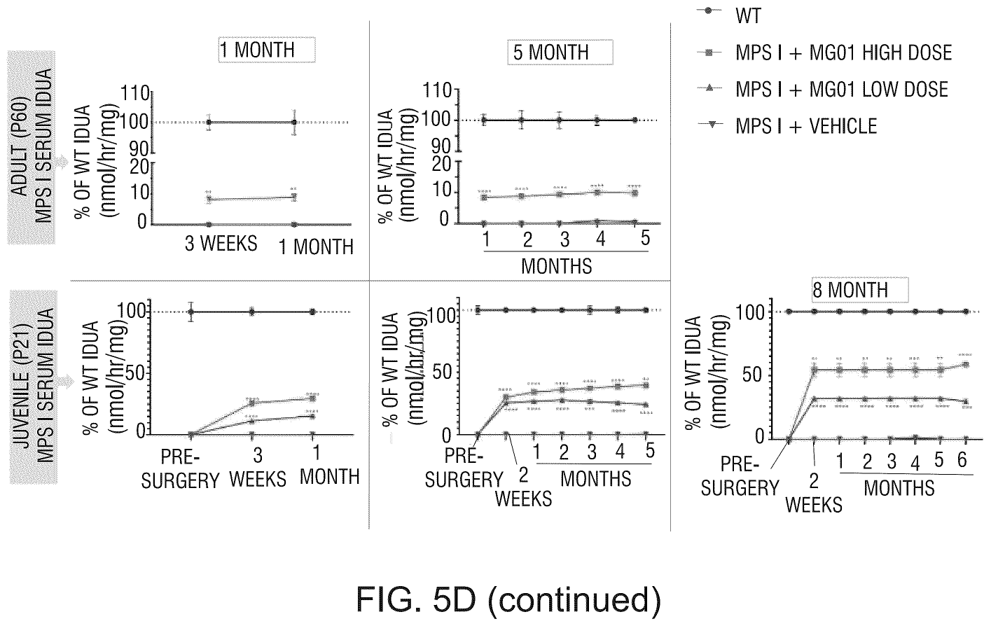

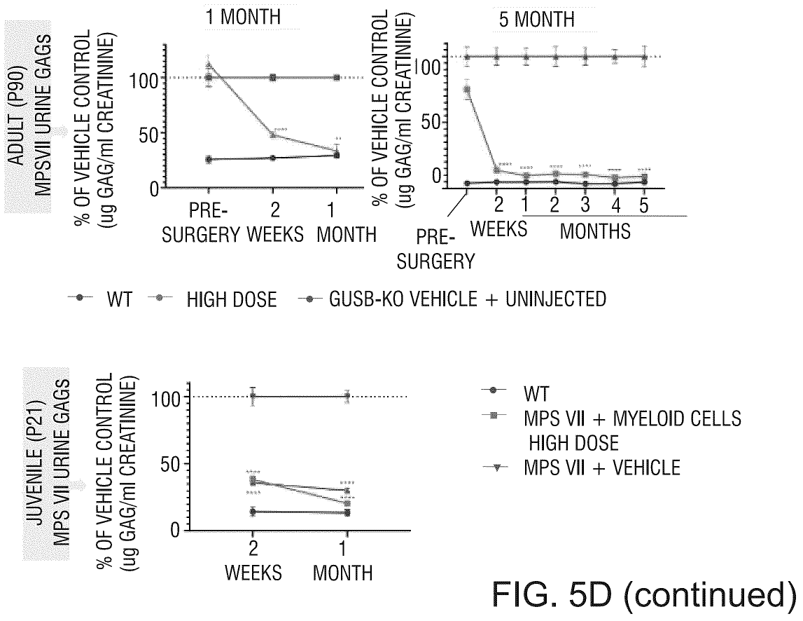

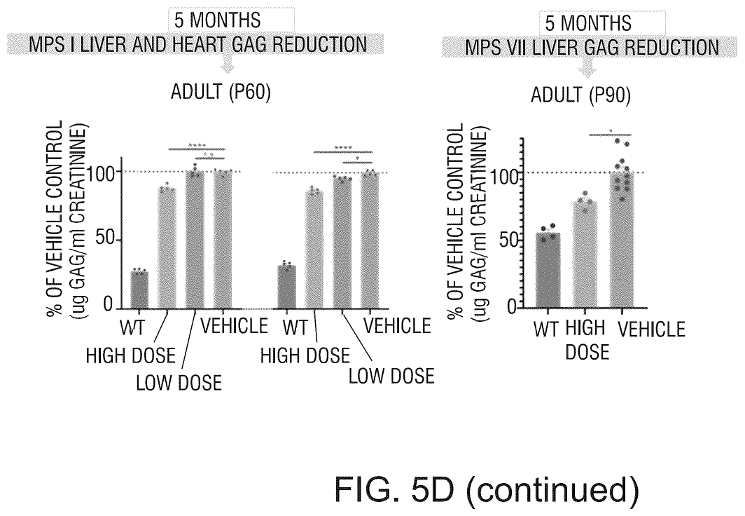

FIGS. 4A-4B shows the biochemical analyses of CNS tissues from IDUA-KO mice 5 months after ICV delivery of myeloid cells and similar results as of 1 month (FIGS. 2A-2B) were obtained. FIGS. 5A-5D show the quantification of the accumulated substrate of IDUA (represented as total GAGs) in CNS tissues of IDUA-KO animals 1, 5 and 8 months after myeloid cell transplantation and similar results as of 1 month (FIGS. 3A-3B) were obtained.

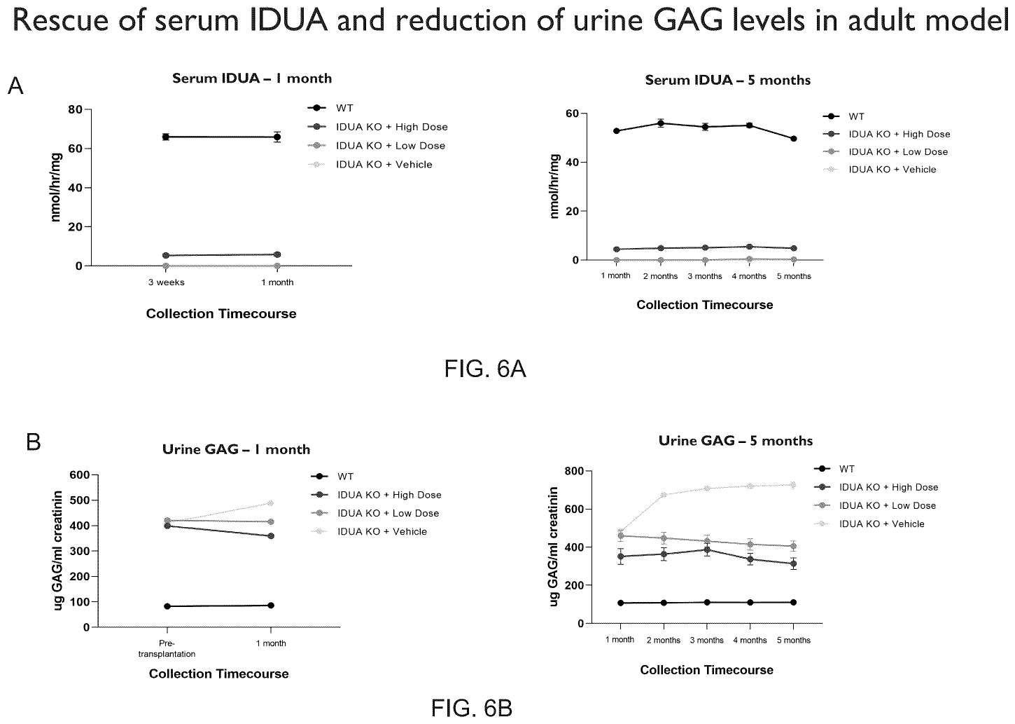

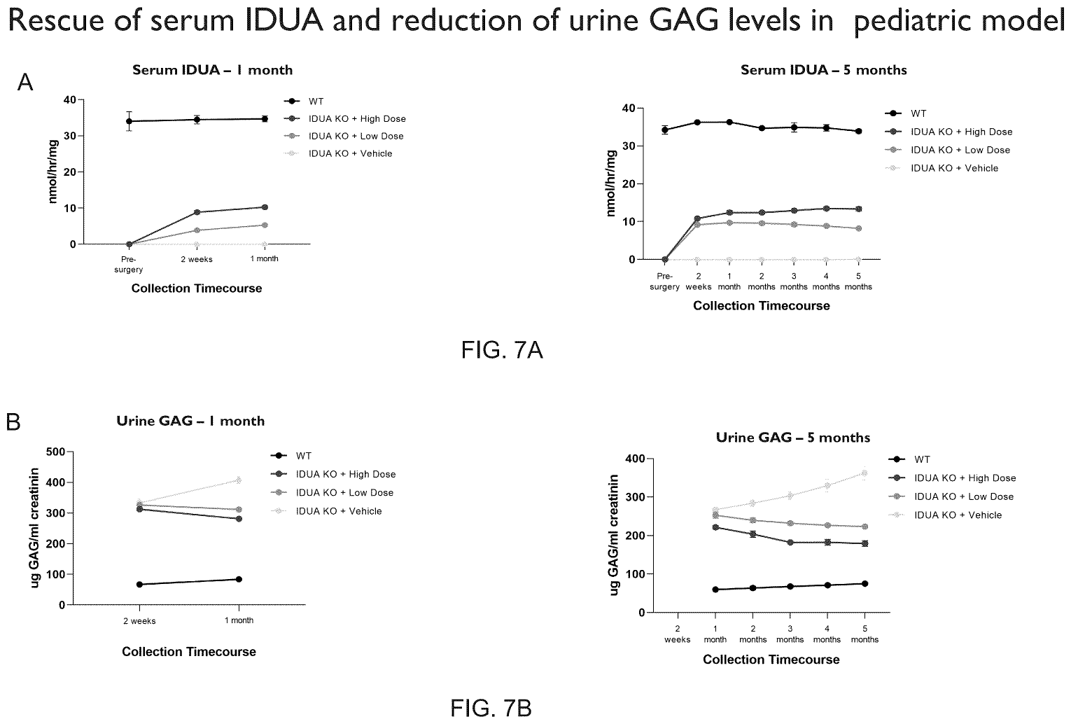

FIGS. 6A-6B shows rescue of serum IDUA and reduction of urine GAG levels in an adult mouse model. FIGS. 7A-7B shows rescue of serum IDUA and reduction of urine GAG levels in a pediatric mouse model.

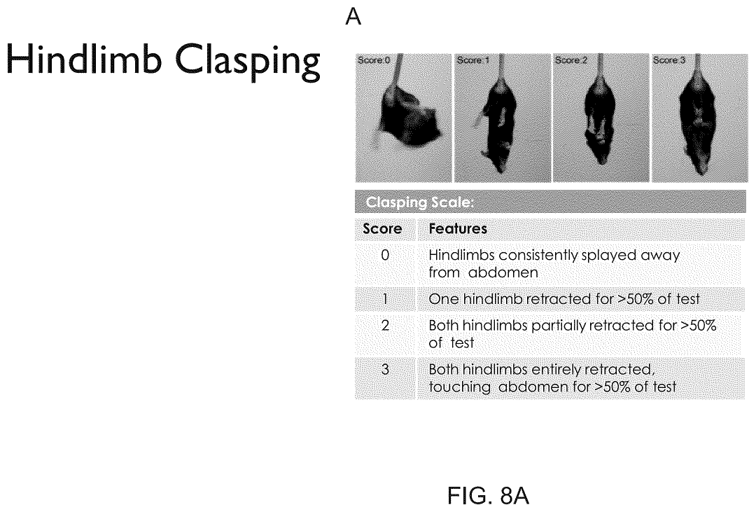

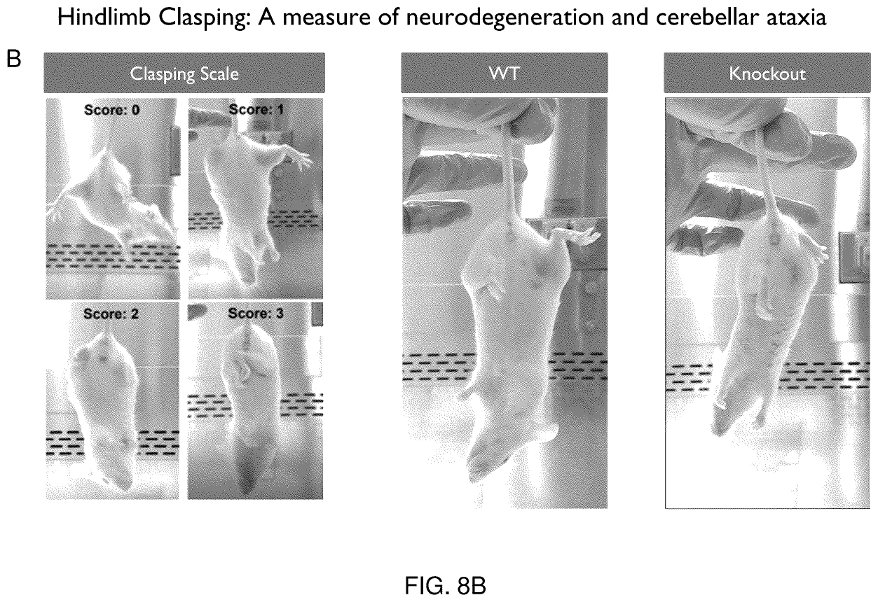

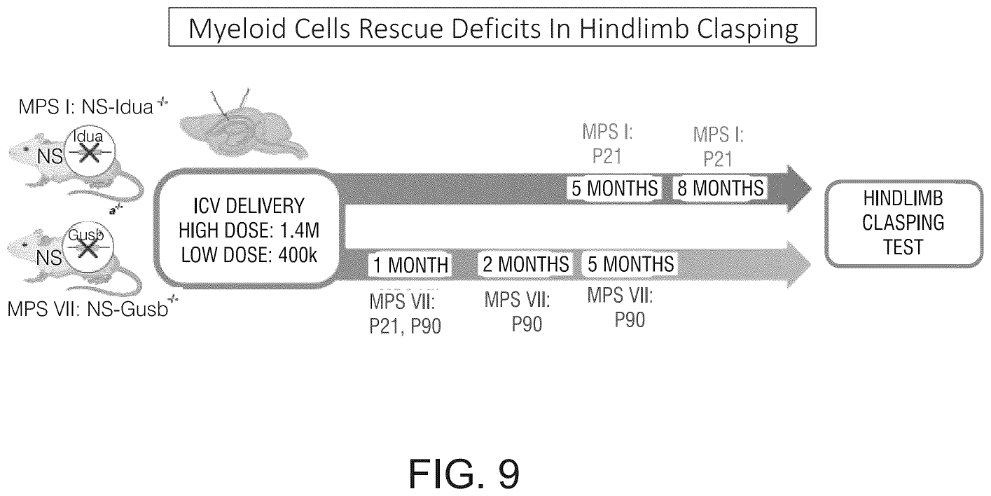

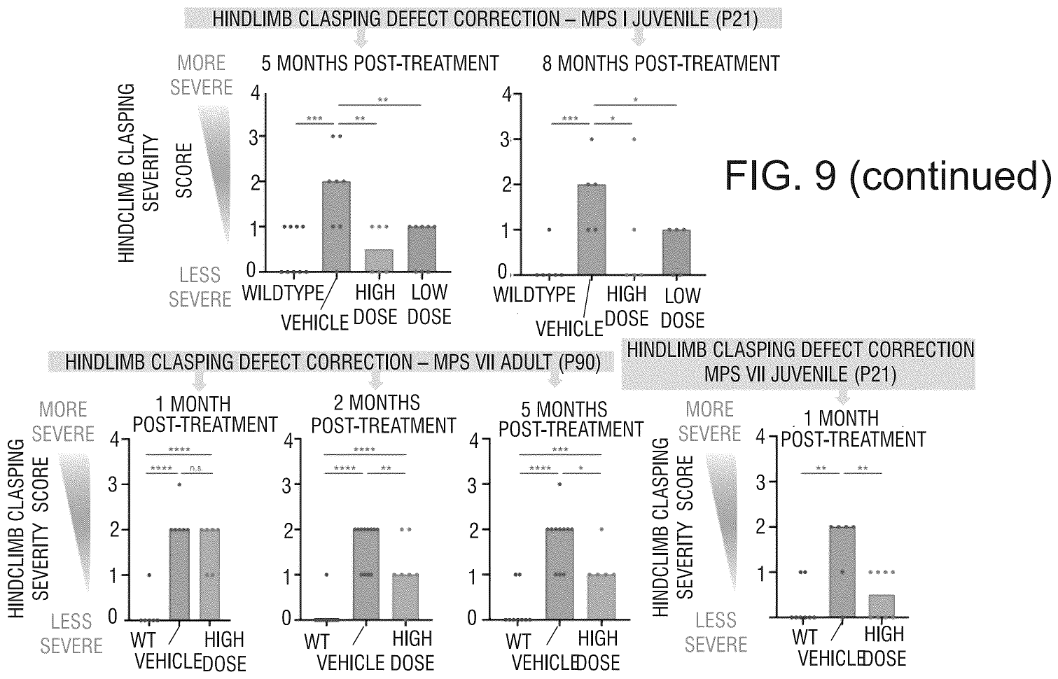

The photos as shown in FIGS. 8A-8B depict mice with different severity scores in the hindlimb clasping test. The table in FIGS. 8A-8B provide further explanation of the scoring. Hindlimb clasping is a behavioral test used as a marker of disease progression in models of neurodegeneration and cerebellar ataxia (Zhu et al. (2016) Toll-like Receptor 4 Deficiency Impairs Motor Coordination. Front. Neurosci.). FIG. 9 shows the hindlimb clasping test in pediatric IDUA-KO animals 6 and 9 months after myeloid cell transplantation. Both the high and the low dose of myeloid cells was able to reverse the behavioral deficit observed in IDUA-KO animals.

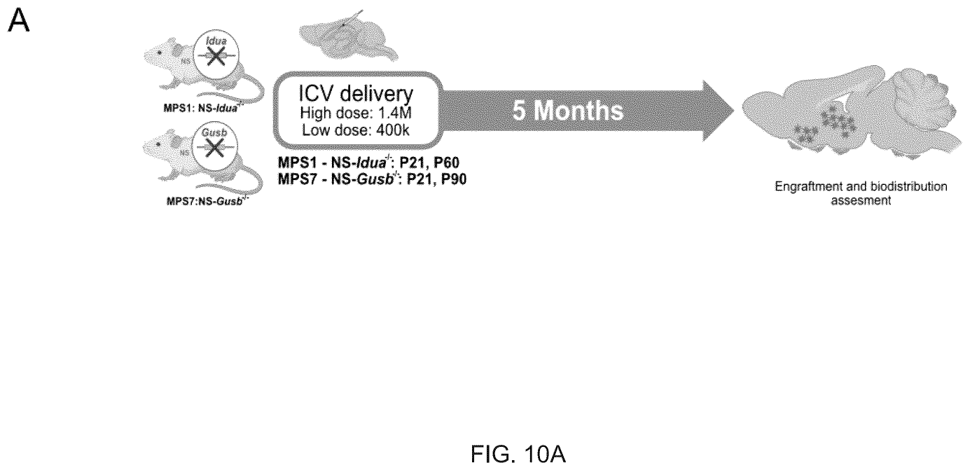

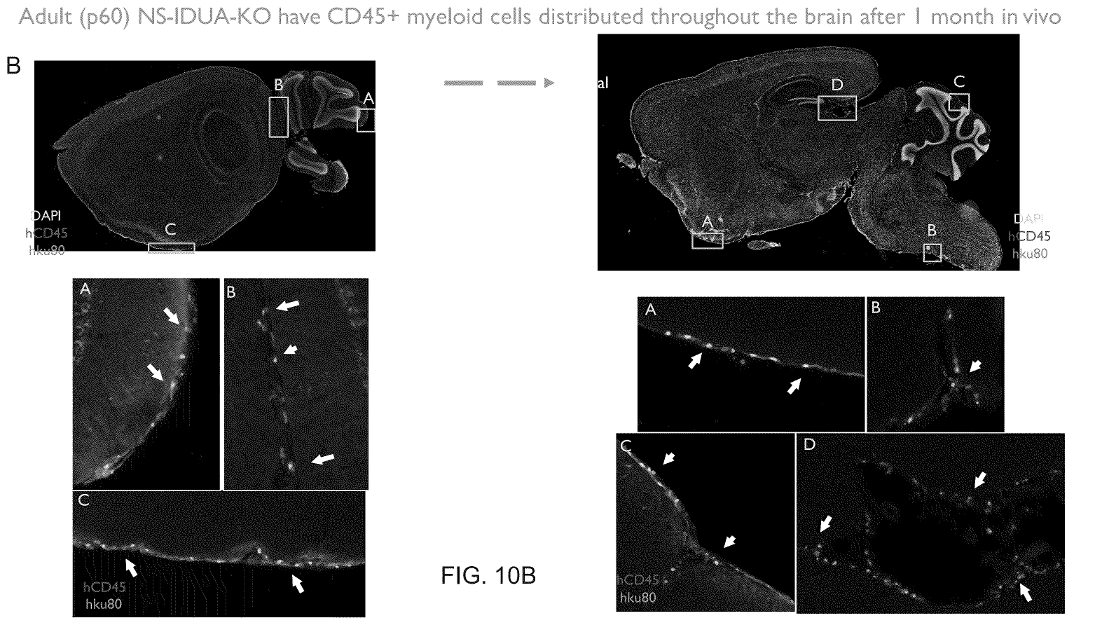

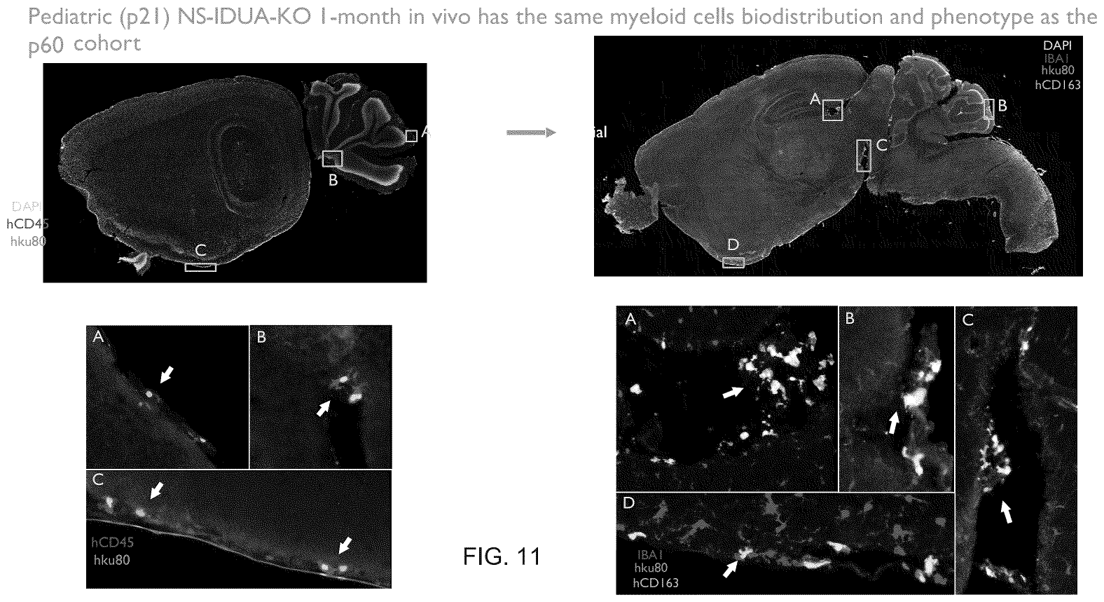

FIGS. 10A-10B and FIG. 11 show immunohistochemistry of two sagittal sections from adult IDUA-KO brain and pediatric IDUA-KO brain respectively, 1 month after ICV delivery of myeloid cells. The results show that both adult IDUA-KO and pediatric IDUA-KO have CD45+ myeloid cells distributed throughout the brain after 1 month in vivo.

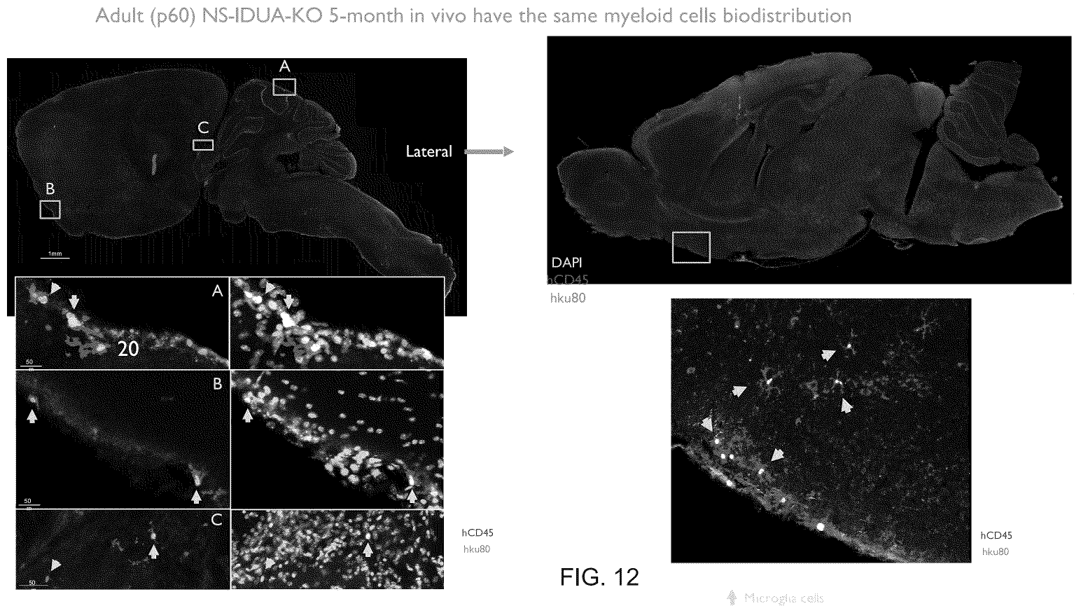

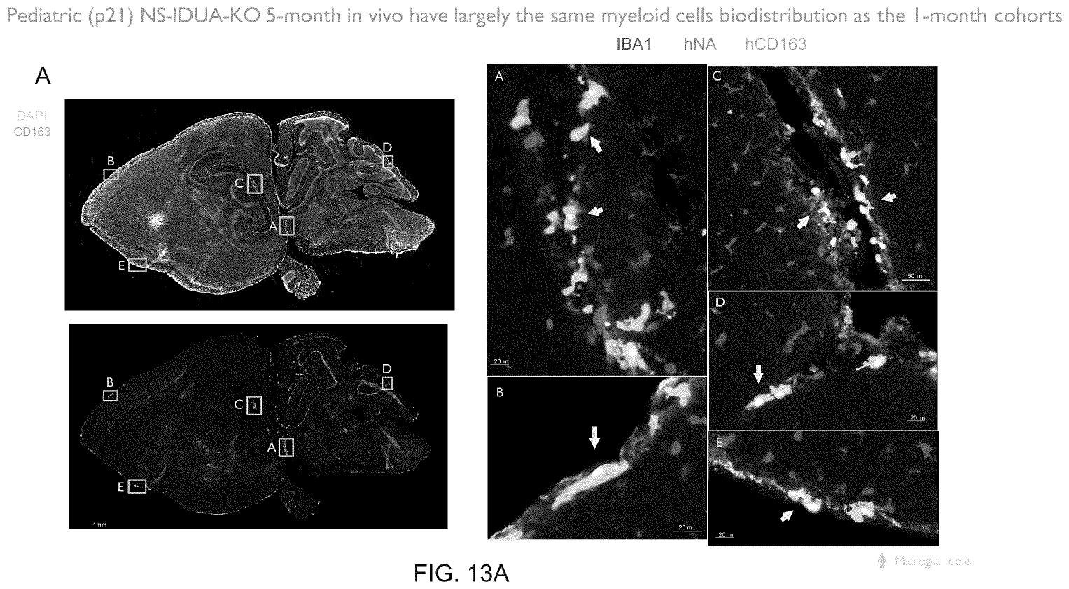

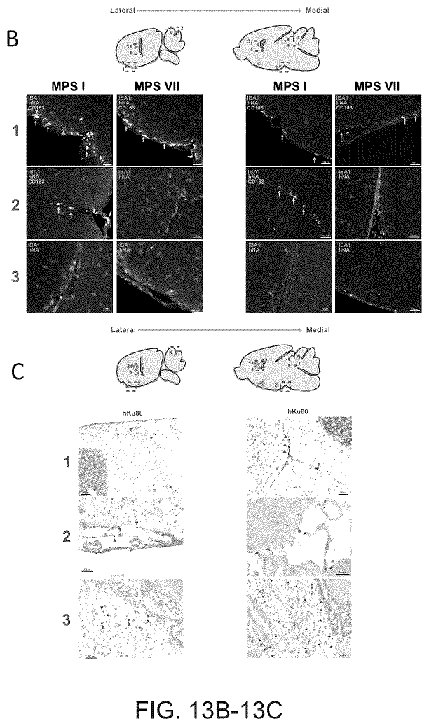

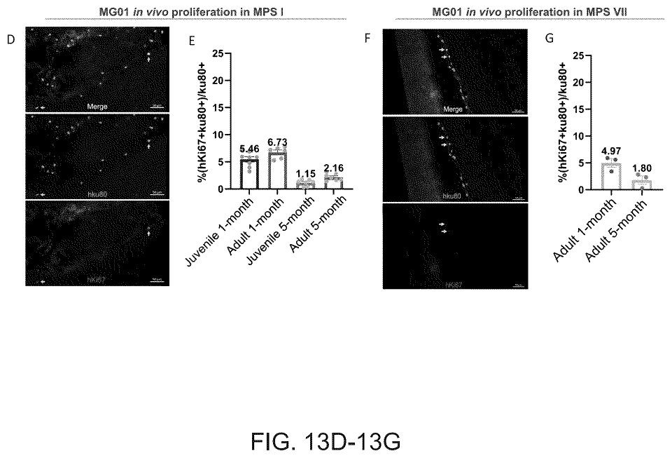

FIG. 12 and FIGS. 13A-13G show immunohistochemistry of two sagittal sections from adult IDUA-KO brain and pediatric IDUA-KO brain respectively, 5 months after ICV delivery of myeloid cells. The results show that at 5-months in vivo, both adult IDUA-KO and pediatric IDUA-KO have largely the same myeloid cell biodistribution as the 1-month cohorts.

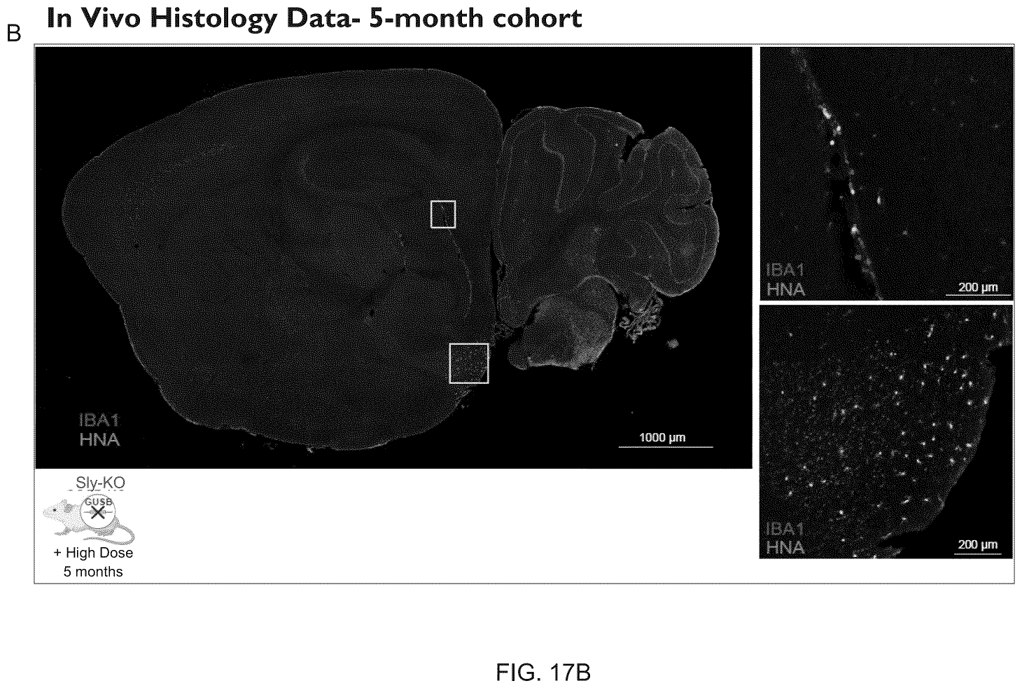

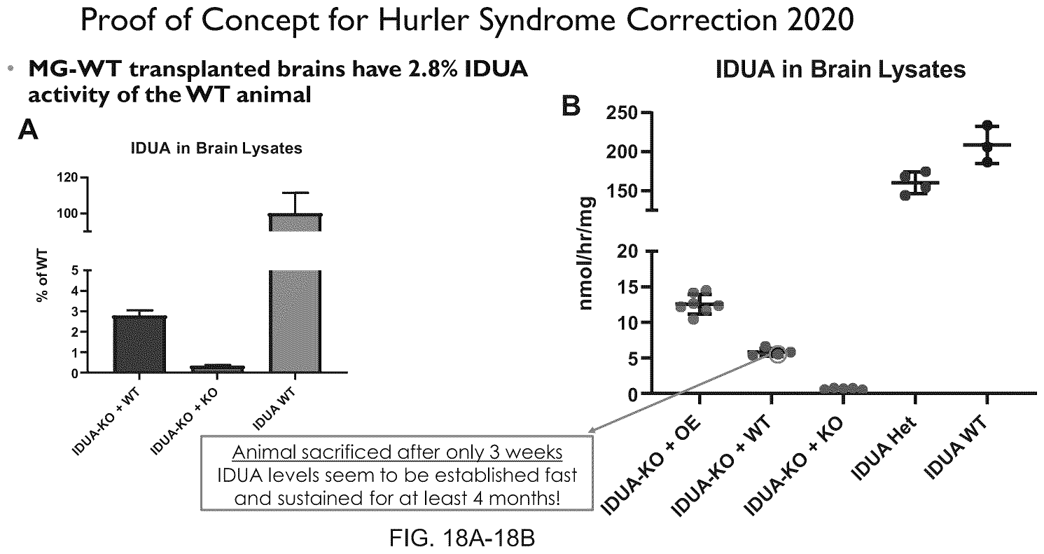

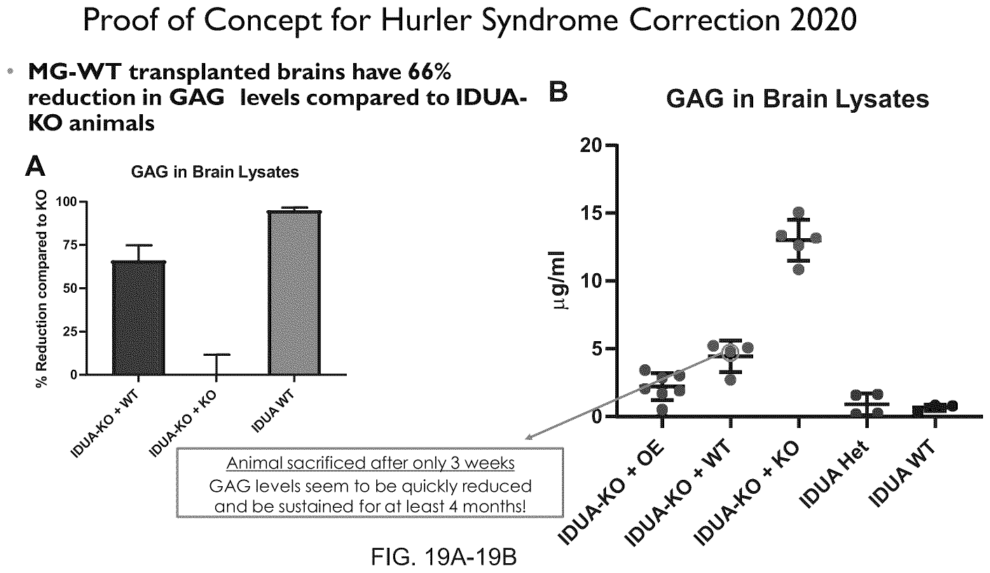

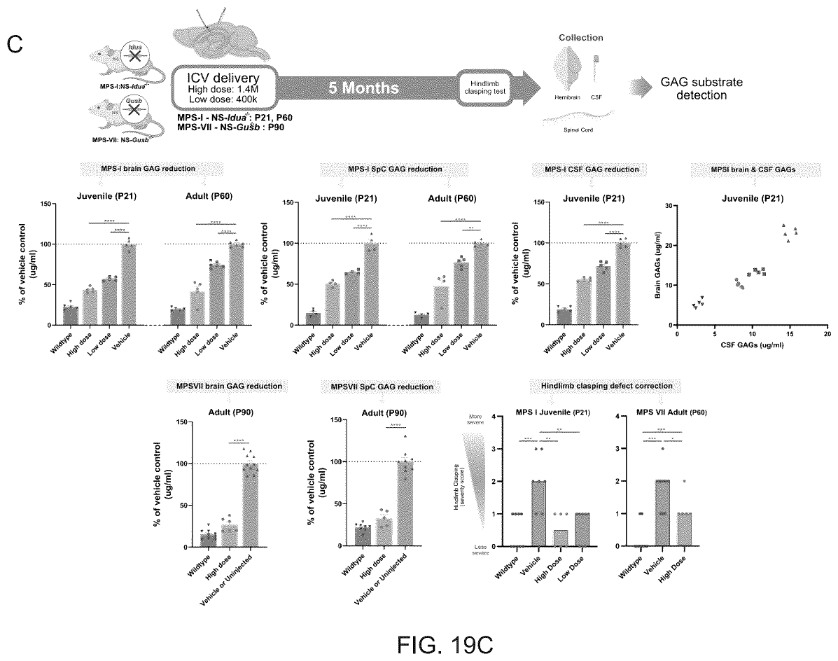

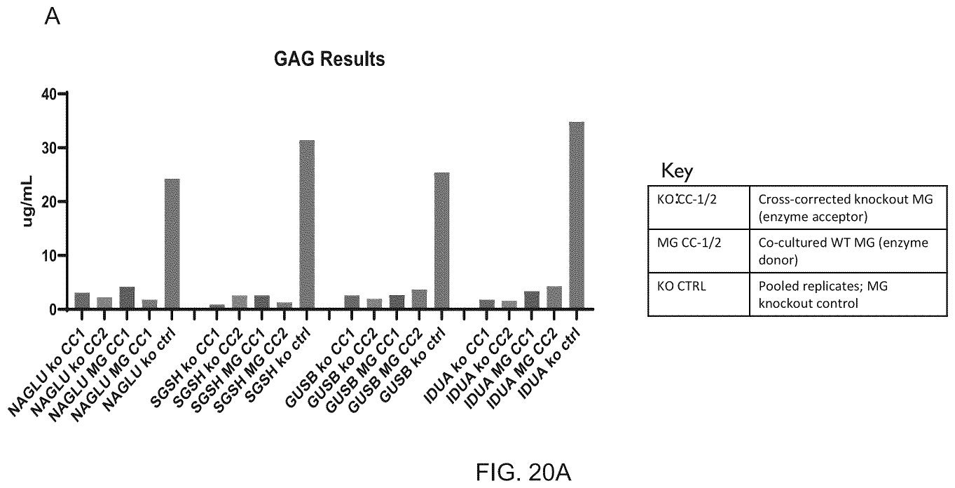

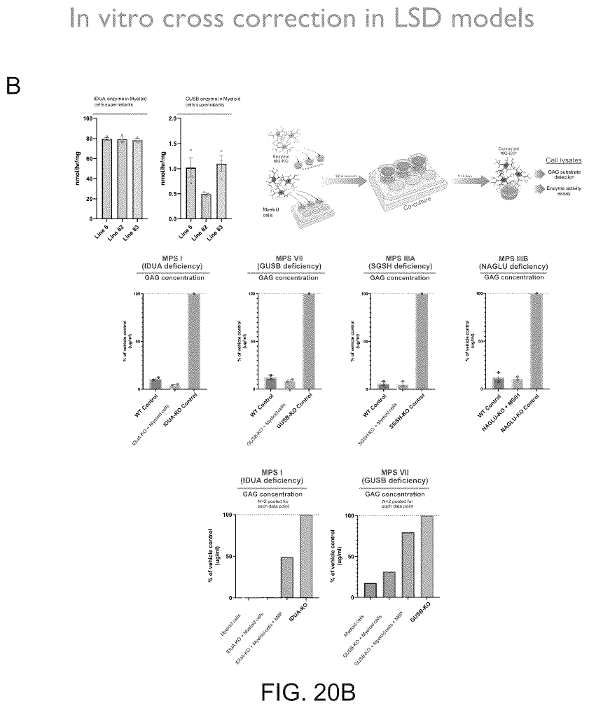

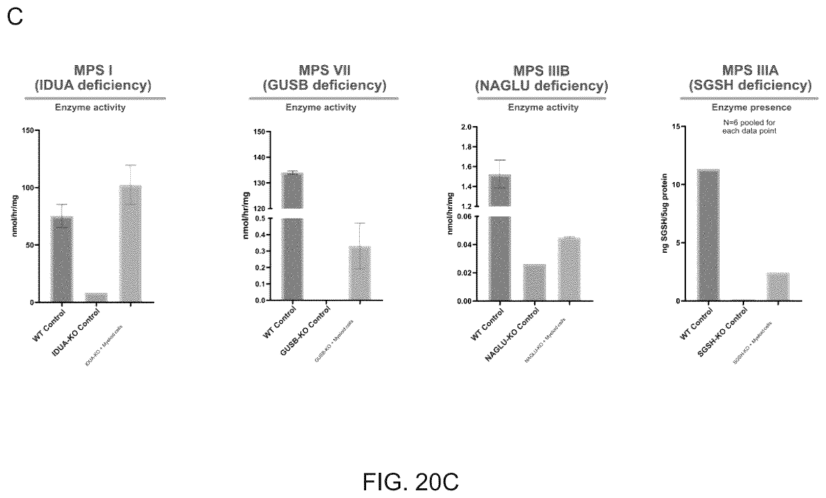

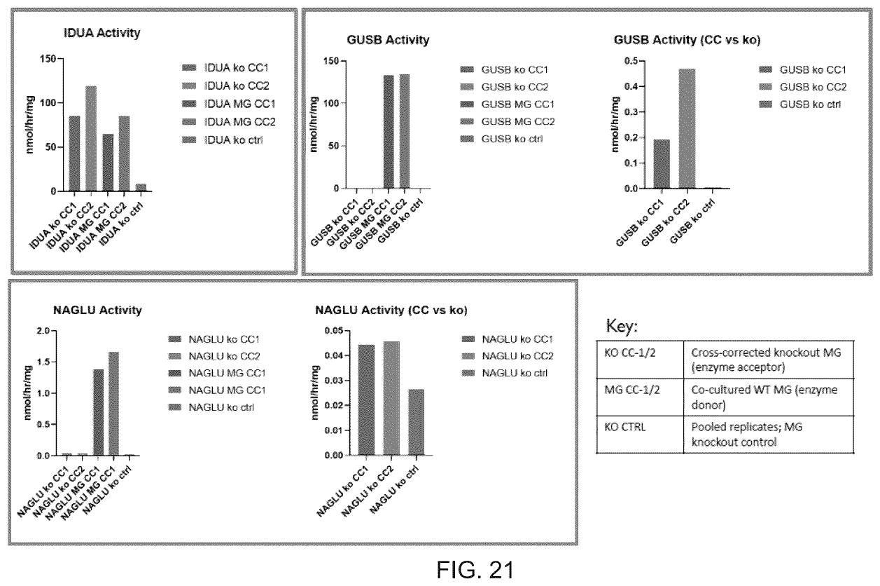

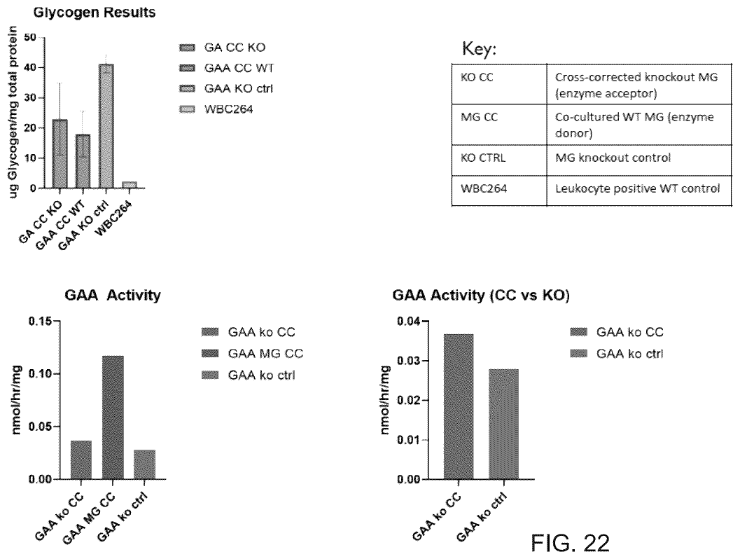

FIGS. 18A-18B show that MG-WT transplanted IDUA-KO mice brains have 2.8% IDUA activity of the WT mice. FIG. 19A. shows that MG-WT transplanted IDUA KO mice brains have 66% reduction in GAG levels compared to IDUA KO animals. FIG. 19B shows that that MG-WT transplanted IDUA KO mice Brains, Spinal Cord and CSF show significant reduction in GAG levels compared to IDUA KO animals at 1 and 5 months (Brain, Spinal Cord and CSF) and at 8 months (Brain and Spinal Cord). These results demonstrate the ability to correct Hurler Syndrome in vivo.

### Sly Syndrome

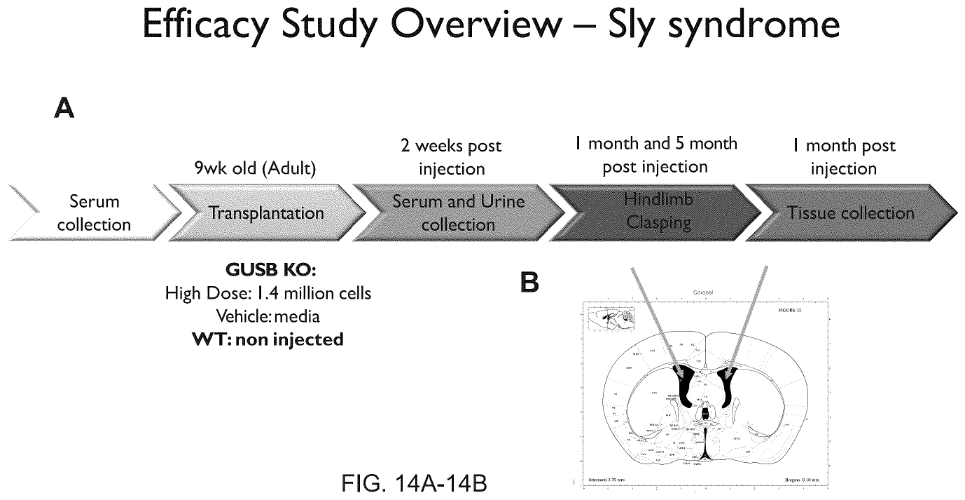

FIGS. 14A-14B show the schematic overview of the non-GMP, in vivo study plan to assess the efficacy of myeloid cell transplantation in the GUSB-KO mouse model of Sly Syndrome. Myeloid cells are transplanted into the brain of 9 weeks old adult IDUA knockout mice. 1.4×10⁶ cells are used for high dose. Coronal section of the mouse brain atlas shows the site of myeloid cell delivery in both lateral ventricles (red arrows). Timeline of the “in-life” phase shows when various samples were collected from the mice and when hindlimb clasping was performed.

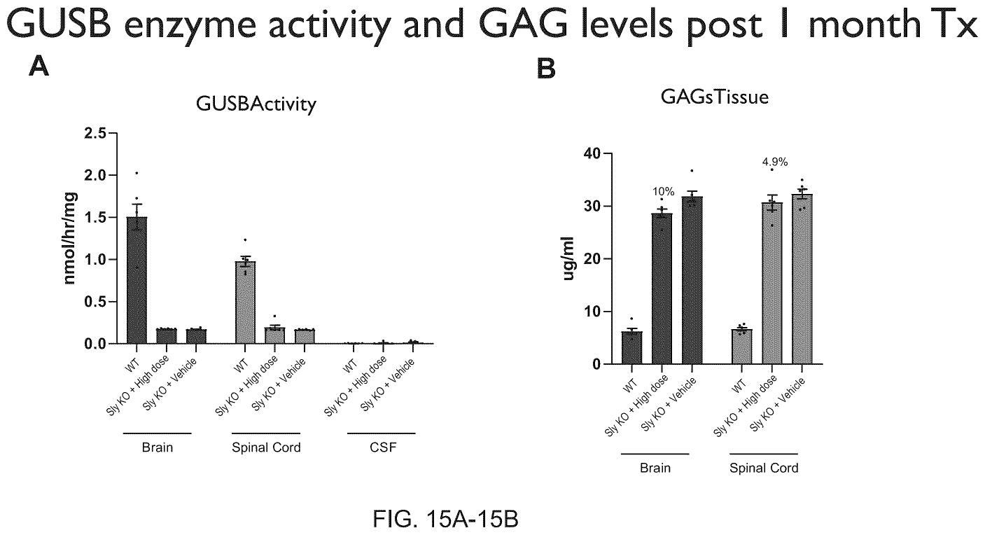

FIGS. 15A-15B show the biochemical analyses of CNS tissues from adult GUSB-KO mice 1 month after ICV delivery of myeloid cells. Myeloid cell transplantation into adult GUSB-KO animals reduced the accumulation of the toxic substrate (represented as total GAGs) in the CNS. Percentages show the reduction of GAGs relative to the vehicle-treated animals.

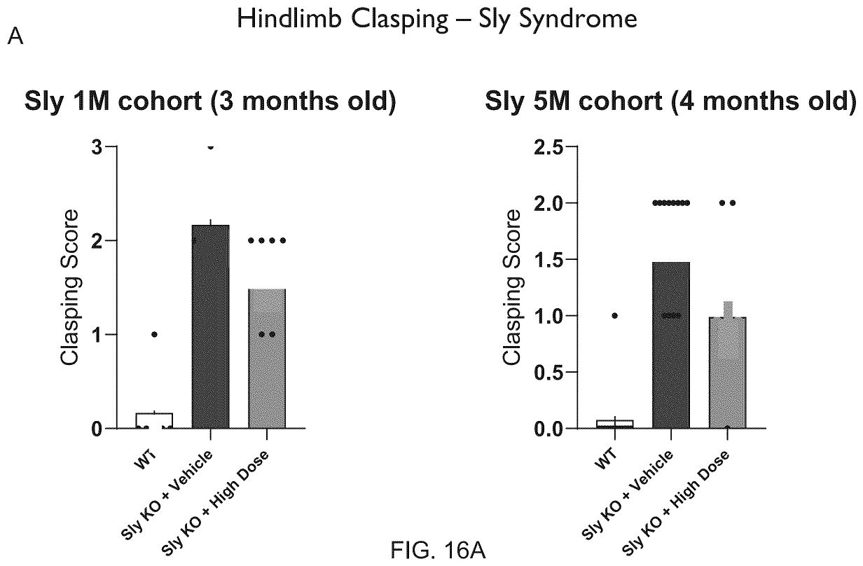

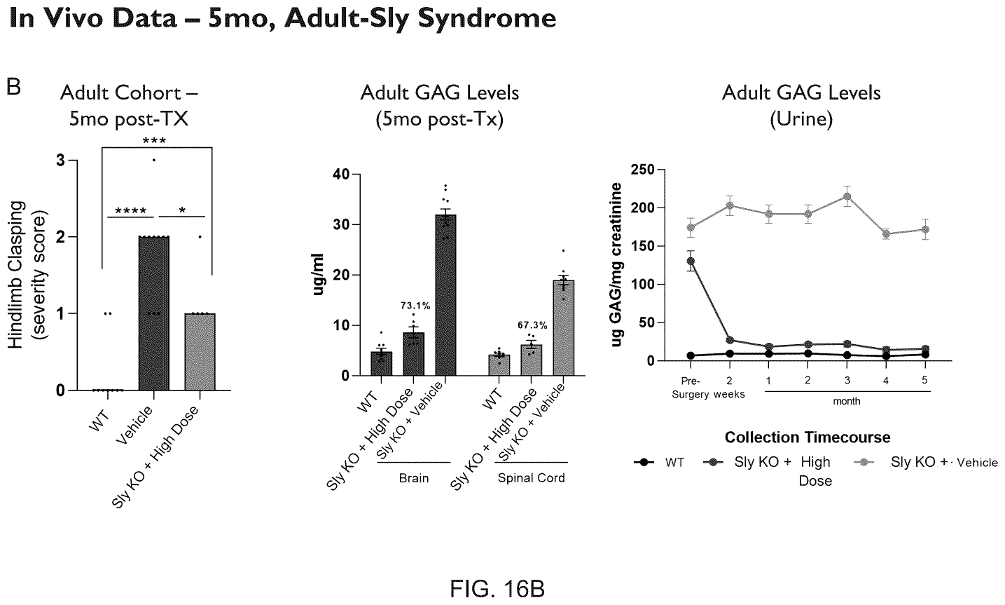

FIGS. 16A-16B show hindlimb clasping test in adult GUSB-KO animals 2 and 3 months after myeloid cell transplantation. Myeloid cell transplantation reduced the behavioral deficit observed in GUSB-KO animals, especially 3 months after transplantation.

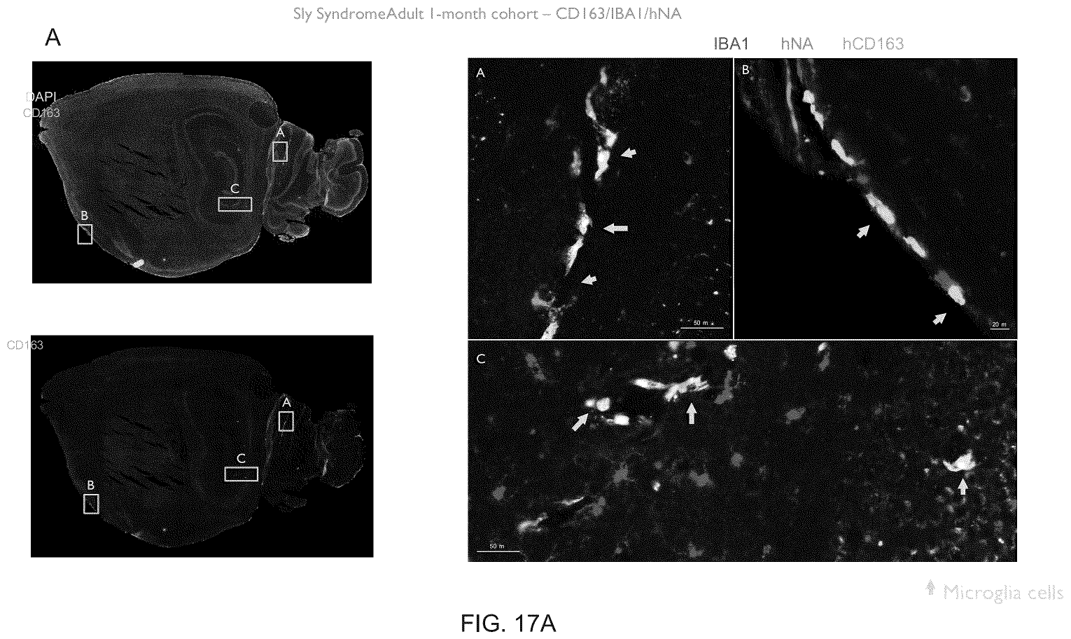

FIGS. 17A-17B show immunohistochemistry of a lateral sagittal section from adult GUSB-KO brain, 1 month after ICV delivery of myeloid cells. Myeloid cells (arrows) were positive for all markers and were found in the choroid plexus and the meninges surrounding the brain.

### Example 4

FIGS. 23A-23B show the generation of genetically engineered hiPSCs line that overexpresses IDUA, to serve payload carriers. The IDUA-OE cell line can contain insertions of cassettes including an aEF1-IDUA cassette into the AAVS1 locus, which leads to sustained expression of high levels of IDUA in two different clones (FIG. 18A). An IDUA knockout line was simultaneously engineered to serve as the target for correction in vitro and as a transplantation control in vivo (FIG. 18B).

### Example 5

NSG-Q mice were developed by crossing NSG-SGM3 with NSG-CSF1 mice they expressed human M-CSF (colony stimulating factor 1 (macrophage)) and the triple transgenic SGM3 transgenes, each with a human cytomegalovirus promoter/enhancer sequence. The CSF1 and SGM3 transgenes encode the human cytokines, which function to support the stable engraftment of human myeloid lineages and regulatory T cell populations. The NSG-Q strain is an immunodeficient mouse model producing human cytokines that can be used to support the survival of human induced pluripotent stem cells (hiPSCs). Brains from NSG-Q mice transplanted with hiPSC-derived microglia generate cells that develop microglial morphology and gene expression patterns similar to primary human microglia. In some brain areas, the donor cells represent up to 50% of the total number of microglia.

Myeloid cells were transplanted in NSG-Q mice at postnatal day 3. The pups were anesthetized and dosed intracerebroventricularly with 140 k cells per hemisphere, for a total of 280 k cells per pup and NSG-SGM3 were used as controls. The pups were left for 3 months to reach maturity and allow time for proper engraftment of myeloid cells.

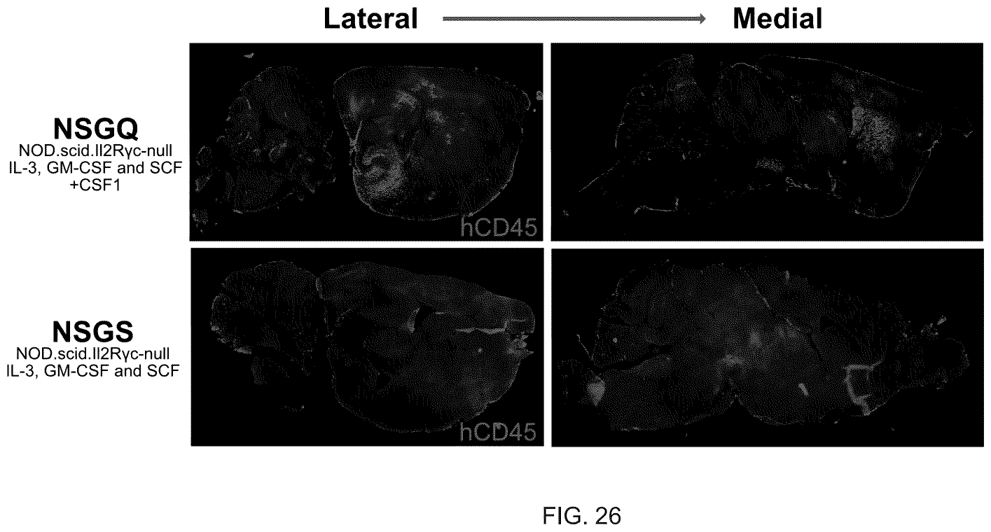

Immunofluorescence staining of the NSG-Q and NSGS reveal that myeloid cells delivered successfully in the lateral ventricles (FIG. 26). Myeloid cells engrafted in the meninges and choroid plexus, as expected based on previous experiments with other mouse models. However, engraftment in the NSG-Q is more widespread in the parenchyma, seen across the full extent of the hemisphere.

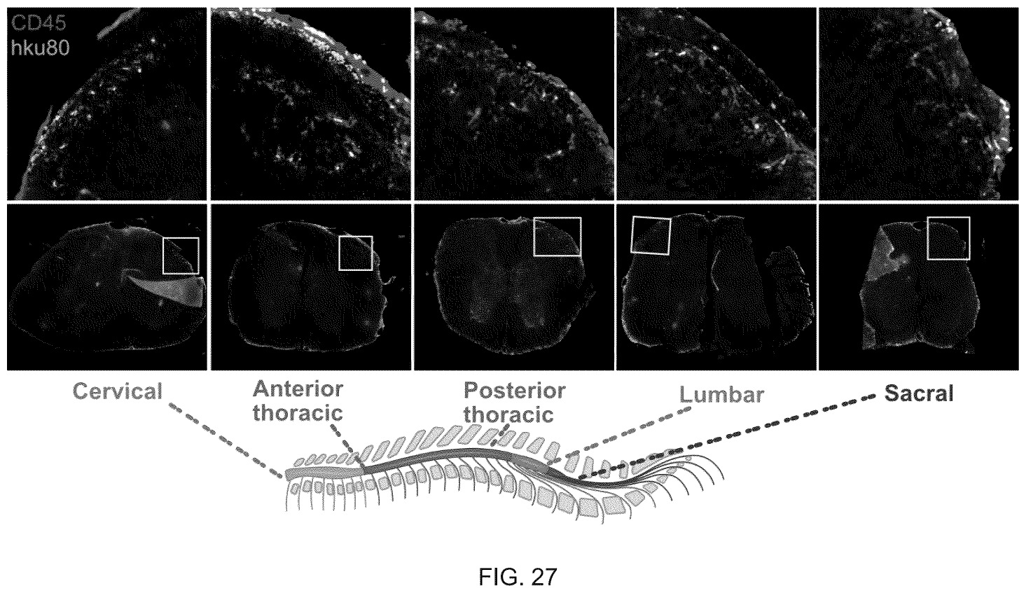

Engraftment of myeloid cells in the NSG-Q extends to the full length of the spinal cord. Engrafted myeloid cells can be detected in the thoracic, lumbar and sacral part of the spinal cord (FIG. 27).

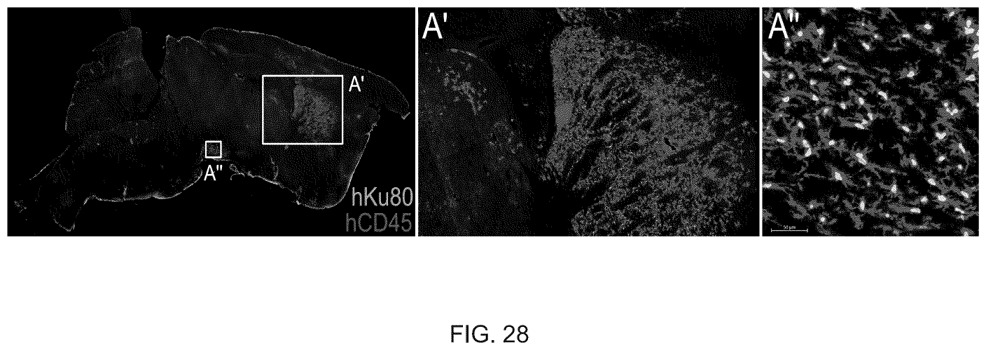

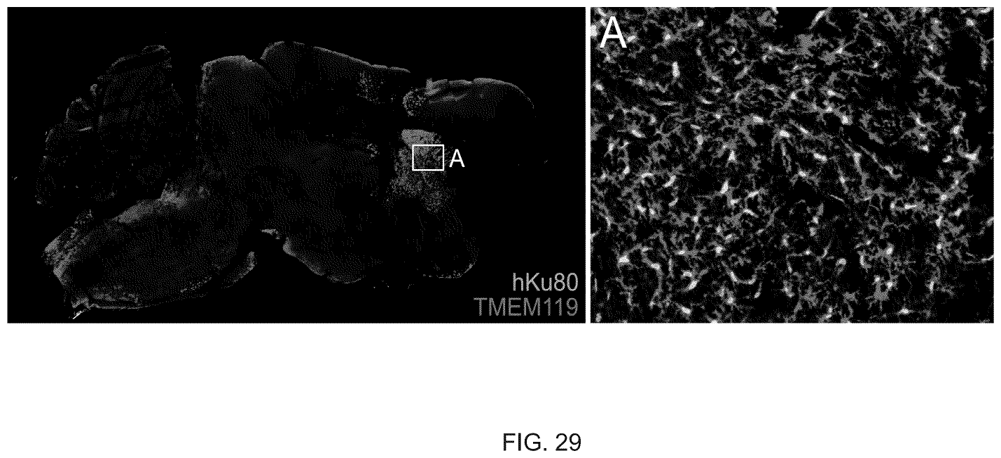

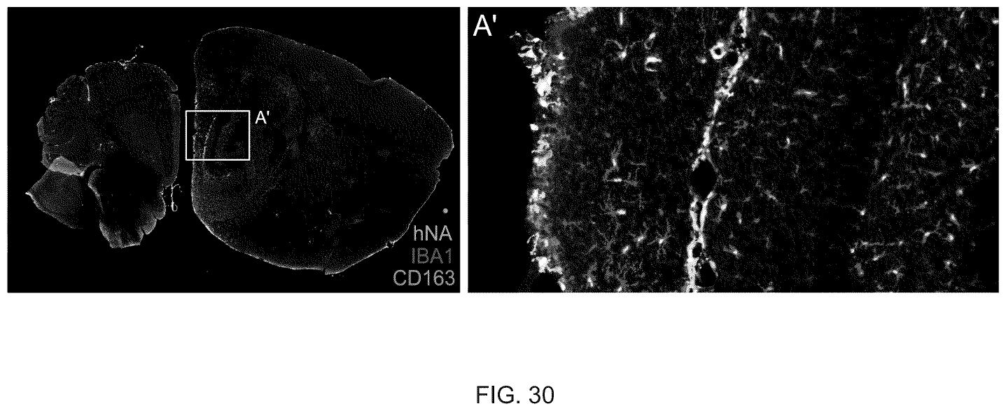

Engraftment of myeloid cells in the brain parenchyma results in a ramified “resting” state morphology of the resulting microglia and high expression of CD45. Ramified myeloid cells engrafted in the parenchyma can be seen in the striatum and ventral midbrain, among many other areas (FIG. 28). Myeloid cells in the brain parenchyma also express the canonical microglia marker TMEM119, which suggests that they have adapted to the niche and assume a resting microglia identity (FIG. 29). Myeloid cells are also positive for IBA1, a known microglia marker. When found in the meninges or choroid plexus, myeloid cells also express the canonical meningeal macrophage marker CD163. (FIG. 30).

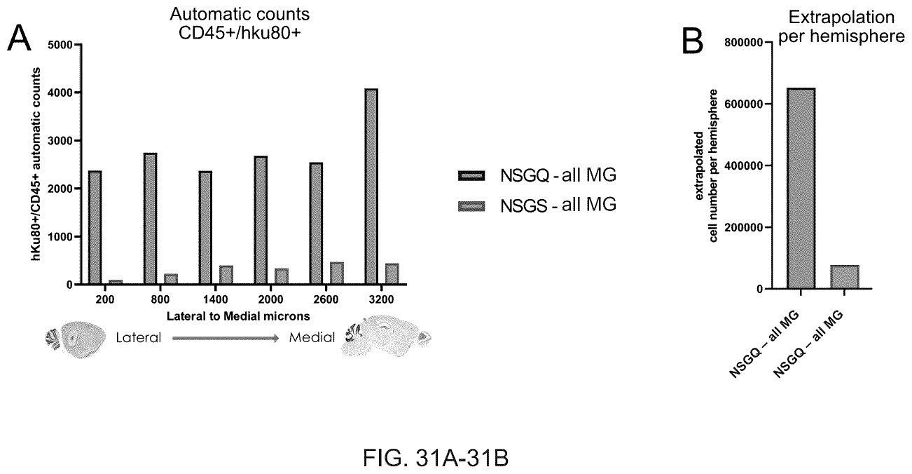

An automated counting method detected the number of CD45+/hku80+ myeloid cells found in mouse brain. There was a 10-fold difference in number of myeloid cells detected in the NSG-Q when compared to the NSGS. The extrapolation was an estimate of what was expected to be present in the whole hemisphere using the sampling counted as the basis for the calculation (FIGS. 31A and 31B).

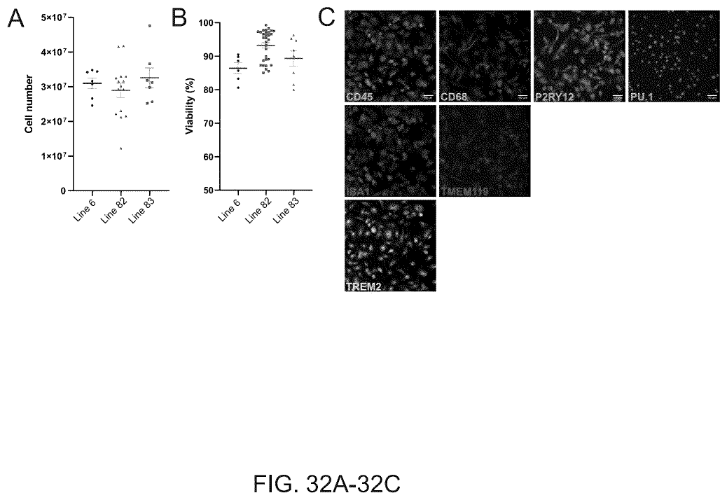

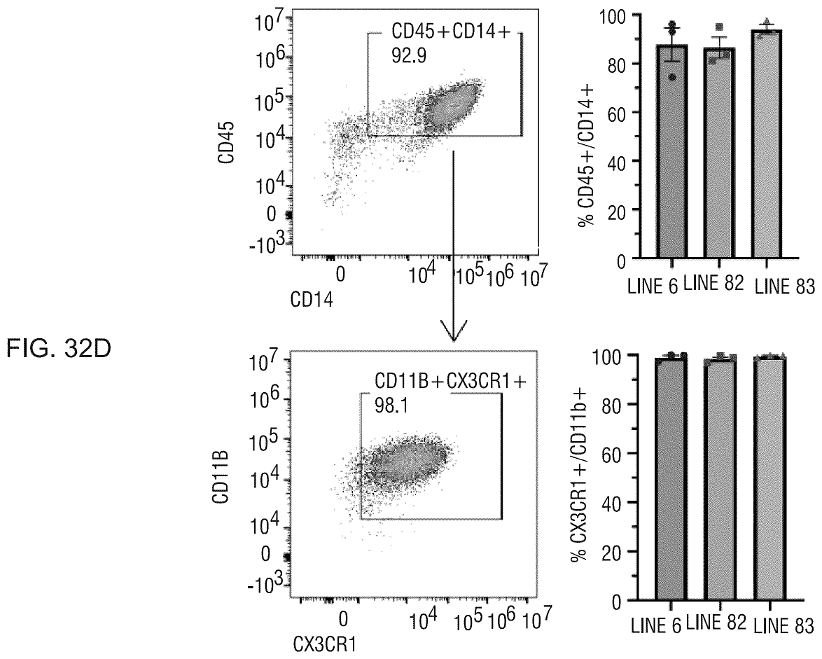

The myeloid cell differentiation protocol is highly efficient, with an average output of 43 myeloid cells for each iPSC and highly reproducible. All 3 lines tested, produce on average 3×10⁷ myeloid cells in a T-75 flask (FIG. 32A). Myeloid cells recovered from cryopreservation, show high viability across 3 hiPSC lines (FIG. 32B) and exhibit canonical microglia markers, including CD45, CD14, CD11B and CX3CR1 (FIG. 32D). Upon plating, myeloid cells exhibit typical microglia morphology and are immunoreactive for microglial markers such as CD45, CD68, TMEM119, IBA1, P2RY12, Pu.1 and TREM2 (FIG. 32C).

Microglia progenitors were isolated and measured on the automated cell counter NC-200 prior to freezing. Cells were resuspended in either BamBanker or STEM-CELLBANKER GMP grade freezing medium and transferred to cryogenic vials. Cryogenic vials with cells were frozen with a controlled rate freezer (CBS CRF2101) using an optimized freezing program. After the freezing program ended, cells were transferred to liquid nitrogen (gas phase) for long-term storage.

To thaw microglia progenitors, cryogenic vial with cells were transferred to a 37° C. water bath for ˜2 minutes until a small ice crystal remained. In a biosafety cabinet, cells from the cryogenic vial were transferred to a centrifuge tube and quenched with 1 mL of either RPMI-1640 or StemPro-34 medium in a drop-wise fashion to reduce osmotic shock. The cells were measured on the NC-200 and quenched with an additional 3 mL of its respective medium. Cells were centrifuged at 250 g for 5 minutes and resuspended in the appropriate assay medium.

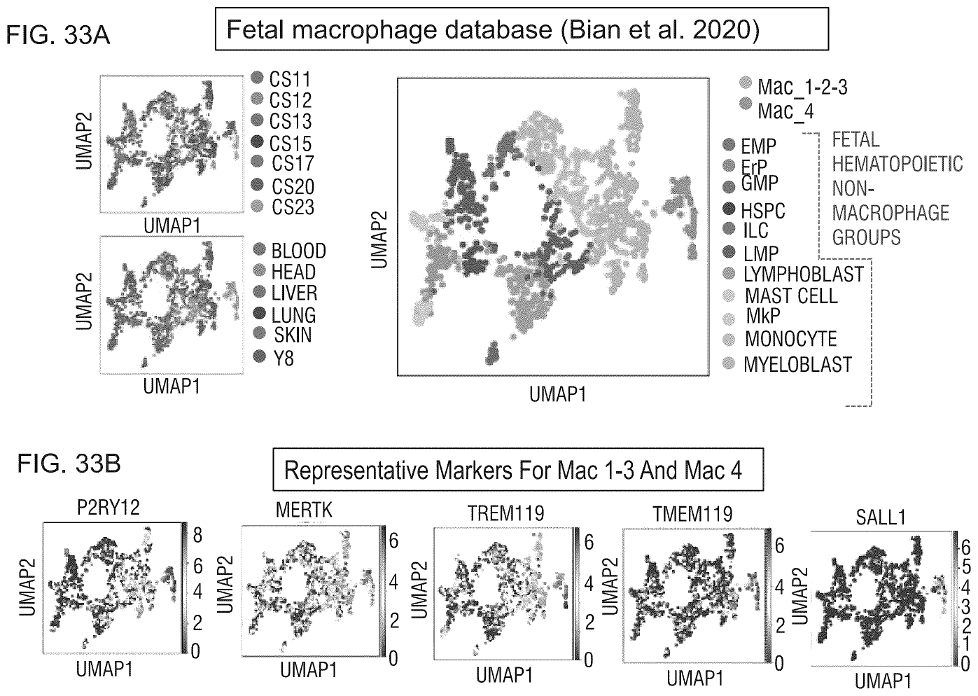

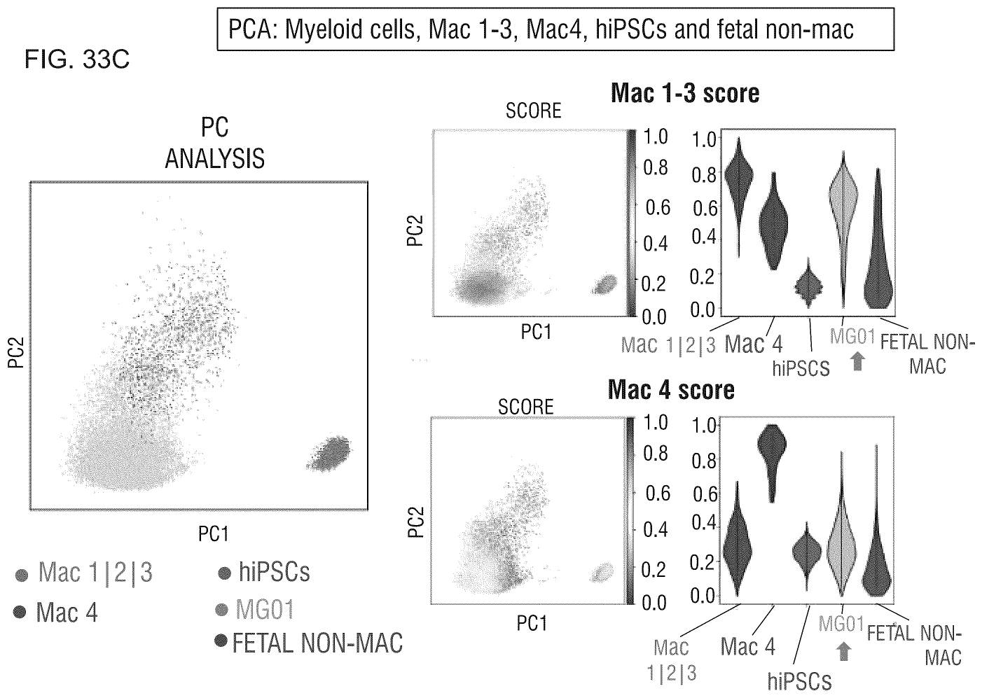

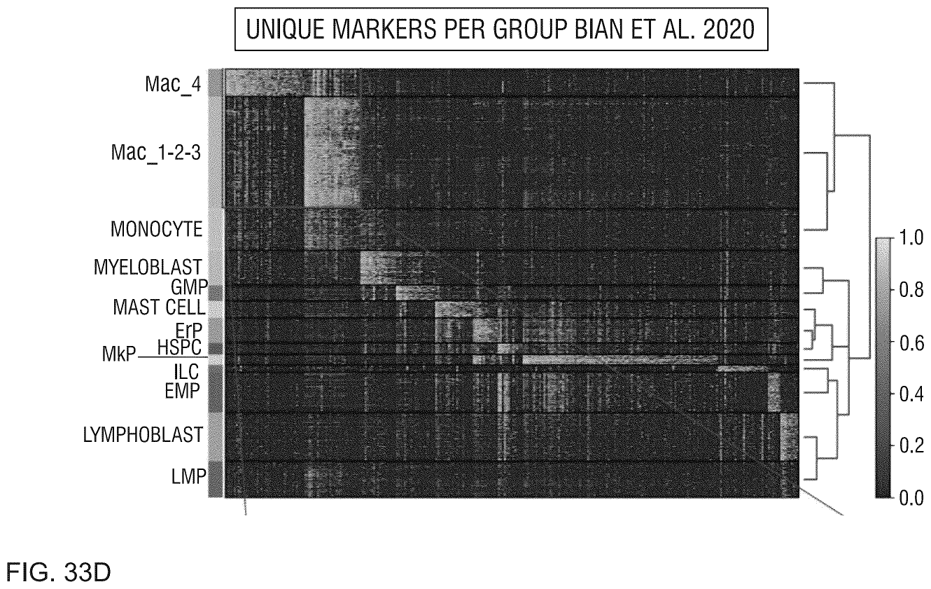

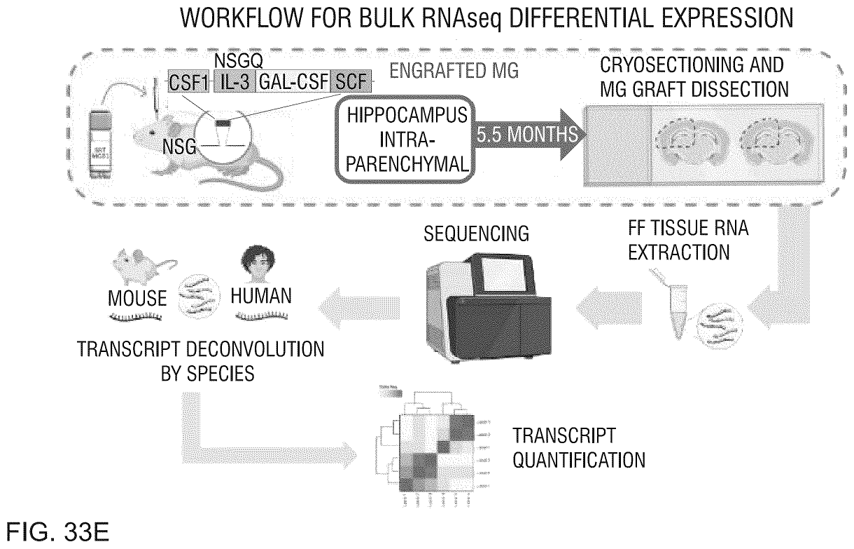

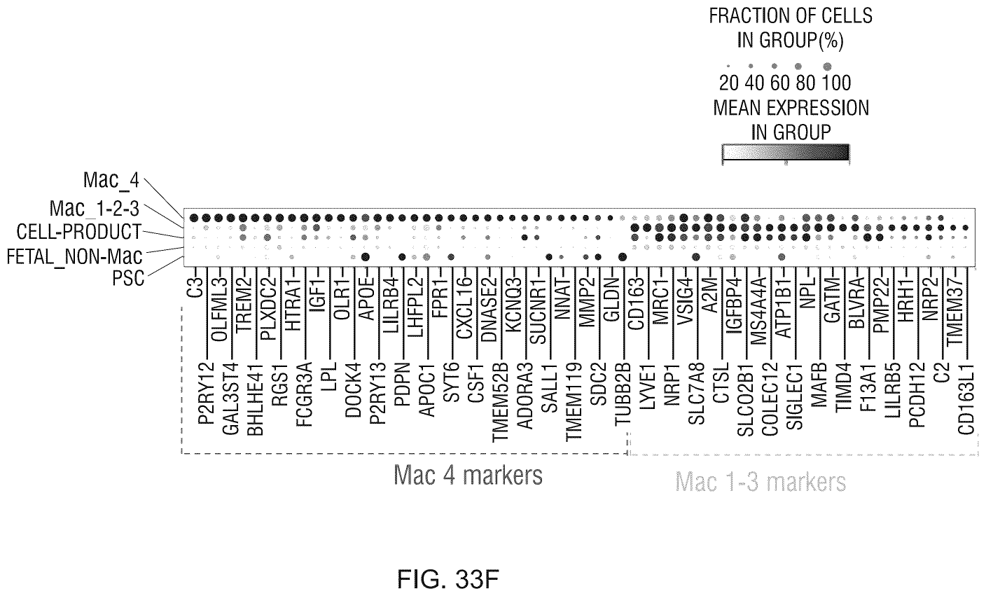

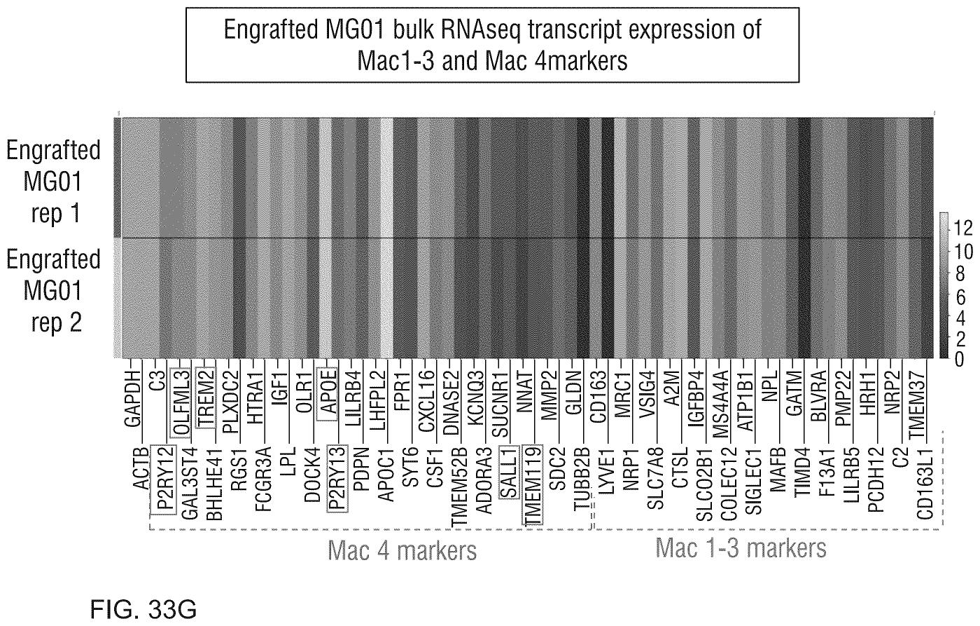

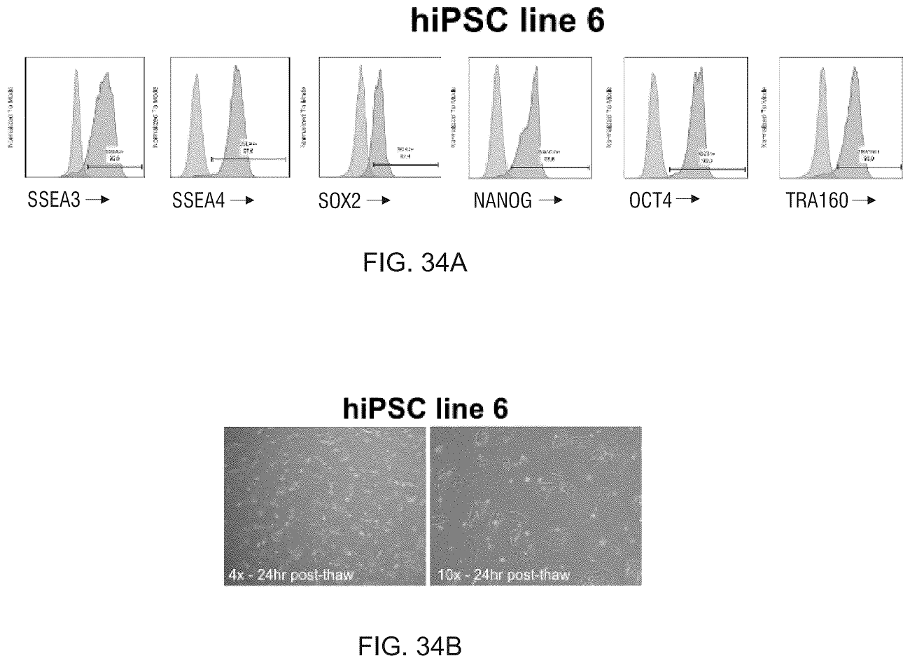

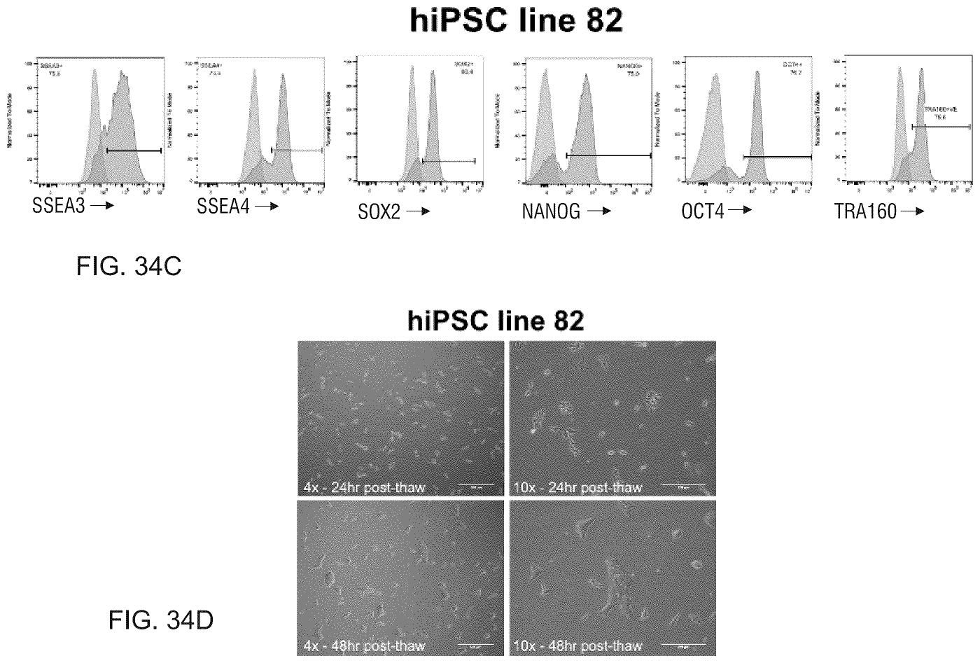

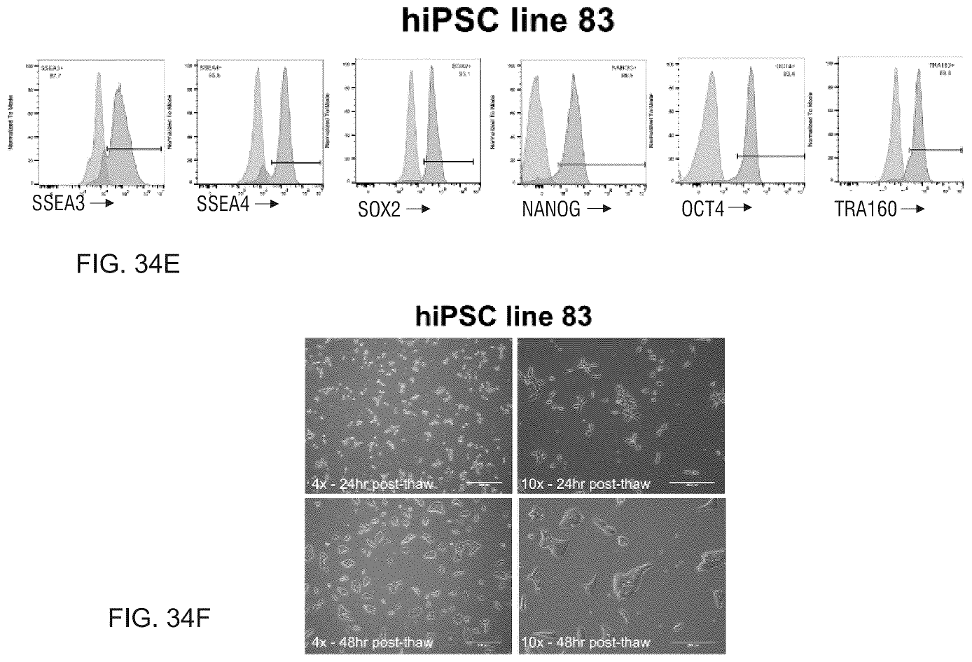

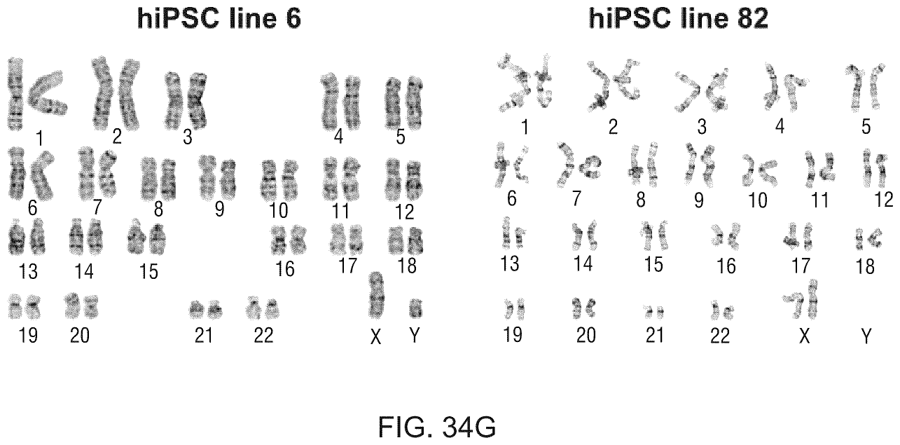

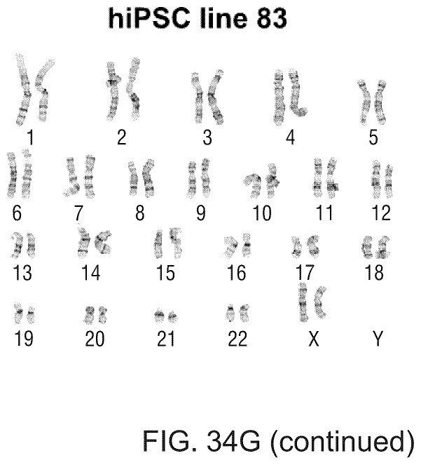

FIGS. 33A-33G shows that the engrafted myeloid cells upregulate microglial signature genes. Single-cell transcriptomic analysis was performed to delve deeper into the molecular profile of myeloid cells before transplantation. As myeloid cells are derived from protocols that mimic microglial development from primitive hematopoietic progenitors myeloid single-cell data was compared to a recent dataset of CD45+ fetal hematopoietic cells (Bian et al. 2020). In the original report, 15 clusters were annotated according to the expression of marker genes with varied representation of Carnegie stages and anatomical origin of sample (FIG. 33A and FIG. 33B). Of those, we found 4 macrophage clusters (clusters 1-3 were treated as one to simplify analysis). Mac 4 shared a macrophage molecular signature with Mac 1-3 but had the addition of unique expression of SALL1 and higher expression of TMEM119, TREM2, MERTK, and P2R1Y12, suggesting a more defined microglia profile.

Using principal-component analysis (PCA), it was observed that myeloid cells clustered closely with Mac 1-3 and Mac 4 while fetal non-macrophage cells (a combination of all other cell identities) lied farther along the PC2 axis. hiPSCs clustered separately from all other cells along the PC1 axis (FIG. 33C). Using the cell signatures derived from the original clusters published in Bian et al. 2020, the identity of myeloid cells was scored, and it was found to be most like Mac 1-3 with a fraction of cells having modest similarity to Mac 4.

To assess changes after engraftment, the differentially expressed genes were utilized for the Mac 4 cluster (FIG. 33D) and combined with differential expression analysis of myeloid cells before and after transplantation in the NSG-Quad (NSG-Q) mouse model, an immunodeficient mouse strain that expresses human cytokines and human growth factors (namely CSF1, CSF2, SCF, IL-3) that support human myeloid cell survival and engraftment 17 (FIG. 33E). An upregulation of Mac 4 markers was detected after engraftment, such as SALL1, TMEM119, MERTK, P2RY12 and TREM2, (FIG. 33F), supporting the microglial identity (and increased similarity to Mac 4) of myeloid cells after engraftment.

In parallel, the changes in known microglial transcripts and genes of interest were looked at between pre- and post-engraftment myeloid cells (FIG. 33G). It was found that expression increased in some microglial genes like HEXB and PU.1 [SPI1]. Some markers were already expressed at high levels in myeloid cells and showed similar or slightly reduced levels after engraftment, like IBA1 [AIF1] or PTPRC [CD45]. Lysosomal enzymes (Neuronopathic MPS enzymes) were also present in either equivalent or increased levels after engraftment, supporting our therapeutic strategy (FIG. 33G).

A critical concern for PSC-derived therapies are potential residual PSCs. POU5F1 [OCT4] transcripts in both the pre- and post-grafting data sets were not detected. To further address this, markers of proliferation were looked at and a 4-fold reduction in MKi67 transcript levels was observed (FIG. 33G).

RNA was isolated from micro-dissected fixed histology sections using the RNeasy FFPE kit and analyzed on an Agilent 2100 Bioanalyzer using Agilent 6000 RNA Nano Kit. cDNA libraries for sequencing were generated using the Collibri 3′ mRNA Library Prep Kit for Illumina. Libraries were quantified using the Collibri Library Quantification Kit and evaluated on the Agilent 2100 Bioanalyzer using the Agilent High Sensitivity DNA Kit. Libraries were sequence on the Illumina NextSeq 500 using the NextSeq 500/550 High Output Kit (75 Cycles). Reads were filtered and trimmed using Cutadapt v3.4 (https://doi.org/10.14806/ej.17.1.200) and aligned with STAR v2.7.8a (PMID: 23104886) to both hg38 and mm39. Aligned reads were sorted and indexed using SAMtools v1.12 (PMID: 19505943) and filtered to detect only human-specific reads using XenofilteR (PMID: 30286710). Reads aligning to expressed genes were counted using HTSeq v0.13.5 (PMID: 35311944) and differential expression analysis performed using DESEQ2 (PMID: 25516281).

To evaluate the efficacy of myeloid cell treatment in a juvenile (postnatal day 7) immunocompromised mouse model of Krabbe disease, the reduction of the toxic accumulation of the undegraded substrates (psychosine) is measured. Analysis of psychosine is performed by Nucrotechnics, using mass spectrometry. Tissues for analysis include brain, spinal cord, CSF, urine and the results are reported in μg/mL.

For gait analysis performed 34 days post transplantation, the animals (mice) are placed on a clear plexiglass runway, opposite end of the dark “goal box”. Animals are allowed 1 minute to walk across the runway to reach the “goal box”. Immediately following the first trial, animals are removed from the runway apparatus and placed back at the beginning of the runway to complete a second gait trial. All trials are recorded from underneath runway and tracking of animal body and paws are done via DeepLab Cut. Ataxic gait will assess width between front paws and hind paws, print position, stride length, speed, and duration of step.

### Example 6

### Cell Line Generation

### Human iPSC Line Generation and Maintenance Culture

The hiPSC lines 82 and 83 were derived by reprogramming peripheral blood mononuclear cells (PBMCs) to induced pluripotent stem cells. PBMCs were sourced from a healthy donor through Be The Match BioTherapies (Minneapolis, MN). Be The Match BioTherapies is a cell collection facility that complies with HCT/P regulations and is registered with the FDA. Erythroblasts were isolated and cultured from PBMCs according to Perriot et al (Perriot, S., Canales, M., Mathias, A. & Du Pasquier, R. Generation of transgene-free human induced pluripotent stem cells from erythroblasts in feeder-free conditions. STAR Protoc 3, 101620 (2022). Erythroblasts were electroporated with a synthetic self-replicating Venezuelan equine encephalitis (VEE) RNA replicon as described by Yoshioka and colleagues (Yoshioka, N. et al. Efficient Generation of Human iPSCs by a Synthetic Self-Replicative RNA. Cell Stem Cell 13, 246-254 (2013). The VEE replicon is a positive-sense, single-stranded RNA that does not utilize a DNA intermediate, so there is no potential for genomic integration and the absence of viral structural genes renders the RNA incapable of viral packaging and pathology. Human iPSC line 6 was obtained from Lonza (Baghbaderani, B. A. et al. cGMP-Manufactured Human Induced Pluripotent Stem Cells Are Available for Pre-clinical and Clinical Applications. Stem Cell Reports 5, 647-659 (2015). iPSCs were maintained using Essential 8 medium (Thermo Fisher Scientific, A26559-01) with recombinant laminin (Biolamina, LN521-05) as the hiPSC attachment substrate. Cell passaging was performed using EDTA as non-enzymatic method (Thermo Fisher Scientific, AM9260G). Dissociated cells were plated onto Laminin 521 coated tissue culture vessels in Essential 8 medium supplemented with 10 μM of Y-27632 (Bio-Techne, TB1254-GMP) for the first 24 hr.

### hiPSC Knockout Cell Line Generation

hiPSC lines lacking IDUA, GUSB, NAGLU, and SGSH were generated to test the ability of myeloid cells to in vitro cross-correct this enzyme deficiency. CRISPR/Cas9 single guide RNAs (sgRNAs) targeting a coding exon of the each of these four genes were designed and tested. The sequences of these sgRNAs are listed in Table 3. A vial of the parental hiPSC line was thawed and transfected with individual Cas9:sgRNA ribonucleoprotein (RNP) complex targeting each of the four genes to generate functional knockouts. The transfected cells were plated onto Vitronectin-coated tissue culture vessels in Essential 8 medium containing Y-27632 (10 μM). Clones were manually isolated by picking and then screened for bi-allelic gene KO using PCR primers flanking the sgRNA target site followed by Sanger sequencing. PCR primers used for genotyping are listed in Table 4. Due to the high editing efficiencies observed in similar experiments, for each gene only 96 or fewer clones were screened using this method. Multiple clones of various KO genotypes were identified for each gene and a single KO clone was selected for myeloid cell differentiation. Final genotypes for each KO line are shown under the “Genotype” column in Table 3.

TABLE 3
sgRNA sequences and genotypes for MG-KOs

| Parental | Gene | Clone | sgRNA sequence (5′ to 3′) | Genotype (bi-allelic) |
|---|---|---|---|---|
| Line 82 | IDUA | c.84 | GTTCCAGGGTTTGAGCTGAT | 1 bp “G” deletion |
| | | | (SEQ ID NO: 1) | |
| Line 82 | GUSB | c.52 | ATCGATGAGTGTCCCGGCGT | 4 bp “CGGC” deletion |
| | | | (SEQ ID NO: 2) | |
| Line 82 | NAGLU | c.1 | GGAACATGCCCCGCGAATGC | 1 bp “T” insertion |
| | | | (SEQ ID NO: 3) | |
| Line 82 | SGSH | c.5 | CCTCACGCAGCGGATGACGG | 1 bp “A” insertion |
| | | | (SEQ ID NO: 4) | |

TABLE 4
PCR primers used for genotyping

| | F Primer | R Primer |
|---|---|---|
| Gene | (5′ to 3′) | (5′ to 3′) |
| IDUA | GACGCTGACCGTCCTTCTG | CGCCTGCCCACCAATGTA |
| | (SEQ ID NO: 5) | (SEQ ID NO: 6) |
| GUSB | TCCACGGTGTCAACAAGCAT | GGGTGGTTCTTGTCCCTACG |
| | (SEQ ID NO: 7) | (SEQ ID NO: 8) |
| NAGLU | AACCTGCACACCTGGGATG | TGTGCAAAGTCACAGATA |
| | (SEQ ID NO: 9) | GATCG (SEQ ID NO: 10) |
| SGSH | GATGTACCCACTCGGGTGTC | TCTGCACTCACAGCCATA |
| | (SEQ ID NO: 11) | GG (SEQ ID NO: 12) |

### Myeloid Cell Differentiation Protocol

Human iPSCs were plated onto Vitronectin (Thermo Scientific, A14700) at 1.0×10⁴ cells/cm² in Essential 8 medium (Thermo Scientific, A1517001) containing 10 μM of Y-27632 (Tocris, 1254) for 24 hours. PSCs were cultured for an additional 2 days in Essential 8 medium with daily medium changes before being induced by Essential 6 (Thermo Scientific, A1516401) medium supplemented with 80 ng/mL of BMP-4 (R&D Systems, 314E-GMP-050). BMP-4 induction continued for 4 days with daily medium changes before the cultures were changed to StemPro-34 SFM medium (Thermo Scientific, 10639011) (containing 1×GlutaMAX Thermo Scientific, 35050061) supplemented with 100 ng/mL of SCF (R&D Systems, 255B-GMP-050), 80 ng/mL of VEGF (R&D Systems, 293-GMP-050) and 25 ng/mL of bFGF (R&D Systems, 233-GMP-025) for 2 days with daily medium changes. On day 6 and day 8, the cells were cultured with StemPro-34 SFM medium containing 50 ng/mL of SCF, 50 ng/mL of IL-3 (R&D Systems, 203-GMP-050), 50 ng/mL of M-CSF (R&D Systems, 216-GMP-500), 50 ng/mL of Flt3 ligand (R&D Systems, 308E-GMP-050) and 5 ng/mL of TPO (R&D Systems, 288-TPE-050). Starting on day 10, cells from the supernatant fraction were pelleted, resuspended in the same fresh medium as day 6 and 8 and placed back into their respective vessel. Beginning on day 14, cells in the supernatant fraction were pelleted, resuspended in StemPro-34 SFM medium containing 50 ng/mL of Flt-3, 50 ng/mL of M-CSF and 25 ng/mL of GM-CSF (R&D Systems, 215-GMP-050) and placed back into their respective vessel. Day 14 medium change was repeated every other day until the cells in the supernatant reached a concentration of >1.0×10⁶ live cells per mL. Beginning on day 18 and every other day after, cell counts were performed prior to pelleting the cells in the supernatant for medium exchange to determine the harvest date. In some embodiments, the myeloid cells are cultured from about 1 to about 30 days, from about 2 to about 25 days, from about 2 to about 20 days, from about 2 to about 18 days, from about 2 to about 15 days, from about 2 to about 10 days, from about 2 to about 8 days, from about 2 to about 6 days or from about 2 to about 4 days. In some embodiments, the myeloid cells are at least 90% CD45⁺, at least 91% CD45⁺, at least 92% CD45⁺, at least 93% CD45⁺, at least 94% CD45⁺, or at least 95% CD45⁺. In some embodiments, the myeloid cells are cultured for about 2 days, for about 4 days, for about 6 days or about 8 days, for about 10 days, for about 12 days for about 14 days, for about 16 days, for about 18 days, for about 20 days, for about 22 days, for about 24 days, for about 26 days, for about 28 days or for about 30 days. In some embodiments, the myeloid cells are at least about 70% CD45⁺/CD14⁺/CX3CR1⁺, at least about 71% CD45⁺/CD14⁺/CX3CR1⁺, at least about 72% CD45⁺/CD14⁺/CX3CR1⁺, at least about 73% CD45⁺/CD14⁺/CX3CR1⁺, at least about 74% CD45⁺/CD14⁺/CX3CR1⁺, at least about 75% CD45⁺/CD14⁺/CX3CR1⁺, at least about 80% CD45⁺/CD14⁺/CX3CR1⁺, or at least about 85% CD45⁺/CD14⁺/CX3CR1⁺.

### Cryopreservation and Thawing of Myeloid Cells

Myeloid cells were isolated and counted on the automated cell counter NC-200 (Chemometec) prior to freezing. Cells were resuspended in either BamBanker (Wako Chemicals, 30214681)) or STEM-CELLBANKER GMP grade (amsbio, 11924) freezing medium and transferred to cryogenic vials (Thermo Scientific). Cryogenic vials with cells were frozen with a controlled rate freezer (CBS CRF2101). Cells were transferred to liquid nitrogen (gas phase) for long-term storage. To thaw myeloid cells, cryogenic vials were transferred to a 37° C. water bath for ˜2 minutes until a small ice crystal remained. In a biosafety cabinet, cells from the cryogenic vial were transferred to a centrifuge tube and quenched with 1 mL of either RPMI-1640 (Thermo Scientific, 11-875-101) or StemPro-34 SFM medium in a drop-wise fashion. The cells were counted on the NC-200 and quenched with an additional 3 mL of its respective medium. Cells were centrifuged at 250 g for 5 minutes and resuspended in the appropriate assay or culture medium.

### Replating and Polarization of Myeloid Cells

Myeloid cells were plated at 1.0×10⁵ cells per well of a 96 well tissue culture treated plate in 50 μL of RPMI-1640 (Thermo Scientific, 11-875-101) containing 1×GlutaMAX supplemented with 100 ng/mL of IL-34 (R&D Systems, 5265-IL-010/CF) and 10 ng/mL of GM-CSF (maturation medium). For M0-like microglia (M (GM-CSF, IL34), resting not polarized microglia/macrophage), an additional 50 μL of maturation medium was added directly on top of the cells. For M1-like microglia (M (LPS,IFNg), inflammatory microglia/macrophage), an additional 50 μL of maturation medium supplemented with 200 ng/mL of LPS (Sigma-Aldrich, L4391) and 200 ng/mL of IFNγ (R&D Systems, 285-GMP) was added directly on top of the cells.

### Cytokine Release Assay (FRET)

Spent medium collected from 48 hour polarized microglia were centrifuged at 250 g for 5 minutes and the supernatant were frozen at −80° C. Supernatant were thawed at room temperature and 16 ul were transferred to small-volume 96 well plate for addition of donor and acceptor antibodies from either the IL-6, TNFα, or CXCL10 kit (Cisbio, 62HIL06PEG, 62HTNFAPEG, 62HCX10PEG). Following the manufacturer's instructions, samples were incubated for 2-24 hours and processed along with standards and analyzed on a Clariostar microplate reader (BMG Labtech) using the recommended parameters from Cisbio. Data was interpreted by calculating the ratio of the acceptor and donor emission signals and determining the delta ratio (ratio standard or sample—ratio standard 0).

### Flow Cytometry Analysis

Myeloid cells were thawed in 2 mL maturation medium as previously described and a cell count was performed on the NucleoCounter. Based on the live cell count per mL, 5.0×10⁵ cells were transferred to a 5 mL FACS tube (Falcon). Aliquoted samples of cells were prepared and placed into separate tubes for unstained and viability dye controls. Additional maturation medium was added to the cells at a 1:5 dilution to further dilute the cryopreservation medium. The cells were pelleted by centrifugation at 250 g for 5 minutes, the supernatant was carefully removed, and the cells were resuspended in 10 μL of surface marker staining cocktail shown in Table 5. The unstained sample was resuspended in 100 μL of 1×DPBS (Thermo Scientific, 14190250) and viability dye (Thermo Scientific, NC0476349) control was resuspended in 100 μL of 1×DPBS containing 0.33 μL of the viability dye. The samples were incubated at 4° C. for 30 minutes, washed with 2 mL of 1×DPBS and pelleted by centrifugation at 200 g for 5 minutes. Samples were resuspended in 200 ul of FACs buffer (Table 5) and then analyzed on the CytoFLEX LX (Beckman Coulter), FCS files were exported and gated on FlowJo software.

TABLE 5
Surface marker cocktail for flow cytometry of myeloid cells

| Reagent: | | |
|---|---|---|
| FACs Buffer (1X FBS 10 mL, 1X HBSS | | |
| 490 mL, 0.5M EDTA 2 mL) | Amount per sample: | |
| 1x PBS | 97.5 | μL |
| FcR Blocker | 1 | μL |
| CD14-APC | 1 | μL |
| CX3CR1-PE | 1 | μL |
| CD45-BV421 | 0.33 | μL |
| Zombie Aqua Fixable Viability Kit | 0.33 | μL |

### Phagocytosis of E. coli pHrodo Bioparticles

Myeloid cells were plated at 1.0×10⁵ cells per well of a 96 well tissue culture plate, were polarized to either M0- or M1-like microglia. After 2 days, medium change was performed by including pHrodo Red E. coli Bioparticles (Thermo Scientific, P35361) in both M0- and M1-like microglia maturation medium (see polarization section) at a final concentration of 45.45 μg/mL per well. Cells on the plate were placed into the IncuCyte S3 (Sartorius) within a 37° C. incubator with 5% CO2 and imaged every 2 hours for phagocytic activity. One well with M0 medium including pHrodo Red E. coli Bioparticles only (no cells), and a single well with 10×10³ myeloid cells with M0 medium alone (no pHrodo) were also imaged as controls.

### Immunofluorescent Staining for Imaging

Cells were fixed with cold 4% paraformaldehyde (Thermo Scientific, AAJ61899AK) for 10 minutes at room temperature (RT). After fixation, cells were washed with 3 rounds of 1×DPBS (Thermo Scientific, 14190250) for 5 minutes at RT before being permeabilized with three washes of 1×DPBS containing 0.2% Triton X-100 (Sigma-Aldrich, T8787) for 10 minutes each at RT. Next, 10% normal donkey serum (Jackson ImmunoResearch, 017-000-121) in DPBS was added to the cells for 1 hour at RT, and then cells were stained with primary antibody cocktail in blocking solution at 4° C. overnight. Cells underwent 3 rounds of washes with 1×DPBS containing 0.2% Triton X-100 (DPBS-T) for 10 minutes each at RT the next morning prior to adding the secondary antibody cocktail in DPBS at RT for 1 hour. An additional round of three 10-minute DPBS-T washes preceded a 20-minute incubation with DAPI (Invitrogen, D21490) in DPBS at RT. Lastly, cells were washed with DPBS and stored at 4° C.

### In-Vitro Metabolic Cross-Correction

Wild-type and enzyme-deficient (MG-KO) microglia were thawed separately in maturation medium. Wild-type microglia were plated in 6 well tissue culture treated plates at 1.0×10⁶ in 2 mL of maturation medium. In a separate 6 well tissue culture treated plate holding 0.4 μM porous transparent polyester membrane inserts (Falcon, 353090), MG-KO were plated at 5.0×10⁵ in 1 mL of maturation medium (2 mL of maturation medium was added to the well below to prevent the membrane from drying out). After 48 hours, MG-KO on inserts were transferred and placed on top of wild-type wells, to begin co-culture. Medium change was performed at the start of co-culture and every other day for 10-14 days. Standalone MG-KOs on inserts were cultured alone in parallel, as diseased control. To measure metabolic cross-correction wild-type and enzyme-deficient microglia were harvested separately from the insert and wells, lysed in 1% TritonX-100, and evaluated for intracellular enzymatic activity and total GAG content.

### Dose Formulation for Animal Transplantation

Myeloid cells were thawed and transferred to a centrifuge tube. Prior to performing a cell count on the NucleoCounter, 1 mL of StemPro-34 SFM medium supplemented with 50 ng/mL of Flt-3 ligand, 50 ng/mL of M-CSF and 25 ng/mL of GM-CSF (SP34-d14) was added directly on top in a drop-wise fashion. An additional 3 mL of StemPro-34 SFM-d14 medium was added on top to further dilute the freezing medium. Cells were centrifuged at 250 g for 5 minutes at room temperature and the supernatant was aspirated. The cell pellet volume was determined using a 20-200 μL single channel pipette and transferred to a 1.5 mL microcentrifuge tube. Based on the live cell count per mL, the cell pellet was diluted with additional StemPro-34 SFM-d14 medium for a final formulation of 7×10⁴ (high-dose) or 2×10⁴ (low-dose) live cells per μL. The prepared cells were placed on ice until ready for transplantation.

### Enzymatic Activity Assay

To determine enzyme activity, cells from the membrane insert were lysed in 1% Triton-X 100 containing protease inhibitors followed by assessment of enzyme activity and GAG levels. The method was adapted from Ou et al., 2014 (Ou, L., Herzog, T. L., Wilmot, C. M. & Whitley, C. B. Standardization of α-L-iduronidase enzyme assay with Michaelis-Menten kinetics. Mol. Genet. Metab. 111, 113-115 (2014). Enzyme catalytic activity for each lysosomal enzyme was determined by quantifying 4-Methylumbelliferyl (4-MU), the fluorescent moiety produced after cleavage of an artificial fluorogenic substrate that is unique to each enzyme (Table 6). The resulting fluorescence of cleaved substrate was read on the CLARIOstar with an excitation setting at 355 nm and emission at 460 nm. Enzyme levels and activity were interpolated using a 4-MU standard curve. For animal experiments, frozen tissue was homogenized using 1% Triton X-100 in HBSS (Cytiva, SH30588.01) with Halt protease inhibitor (Thermo Scientific, 87786). A BCA protein assay was performed on the resulting lysates to estimate the amount of protein isolated. Up to 50 μg of total protein was used to measure enzyme activity and GAG levels from brain lysates, spinal cord lysates and serum. For CSF, 1 μl of the sample was used for the assay and results were post-normalized to total protein concentration.

TABLE 6
Fluorogenic substrate for MPS enzymes

| | | Involved | |
|---|---|---|---|
| | | degradation | |
| Disease | Gene/Enzyme | pathway | Artificial Fluorogenic Substrate |
| MPS I (Scheie, Hurler | IDUA (Iduronidase) | DS, HS | 4-Methylumbelliferyl-a-L-iduronide |
| Syndrome) | | | |
| MPS VII (Sly Syndrome) | GUSB (β-Glucuronidase) | DS, HS, | 4-Methylumbelliferyl-beta-D-glucuronide |
| MPS IIIA (Sanfilippo A) | SGSH (Sulfamidase) | HS | — |
| MPS IIIB (Sanfilippo B) | NAGLU | HS | 4-Methylumbellifery1-N-acetyl-alpha-D- |
| | (acetylglucosaminidase) | | glucosaminide |

Glycosaminoglycan (GAG) compromised of; dermatan sulfate (DS), heparan sulfate (HS),
keratan sulfate (KS), chondroitin sulfate (CS), hyaluronic acid (HA)

### GAG Quantification

Frozen tissue was homogenized as above. Quantification of GAGs from the various cell lysates was performed using a cationic dye 1,9 dimethylmethelene blue (DMB) (Sigma Aldrich, 341088-1G) which binds to highly charged sulfated GAGs. The resulting absorbance at 525 nm is proportional to the concentration of GAGs in the sample. Values were interpolated using a GAG standard curve.

### Animal and Husbandry

Experiments were performed with male and female mice (3 weeks and 8 weeks old; n=6-8/age group/genotype) with comparative groups (Prkdc(scid) IDUA^{-/-} versus their wild-type Prkdc(scid) IDUA^{+/+} littermates (Strain #:004083, Jackson Laboratory) and Prkdc(scid) GUSB^{-/-} versus their wild-type Prkdc(scid) GUSB^{+/+} littermates (Jackson Laboratory)). Animals were maintained on a 12-h light-dark cycle (lights on at 6:00 am) at 22-25° C. with ad libitum access to food and water. Mice were housed 2-5 per cage. All experiments were conducted in accordance with the guidelines of the Institutional Animal Care and Use Committee.

### Stereotaxic Surgery

30 minutes prior to surgery, animals were injected subcutaneously with 4 mg/kg Meloxicam SR (Putney, RXMELOXICAM-INJ) and were anaesthetized by inhalation of ˜2% isoflurane (Attane, RXISO-250) in oxygen throughout the entire surgical procedure. Anaesthetized animals were positioned in a stereotaxic frame (Kopf instruments) and 10 μL of either myeloid cells low dose (0.4×10⁶ cells/brain), myeloid cells high dose (1.4×10⁶ cells/brain), or vehicle (StemPro-34 SFM medium containing 50 ng/mL of Flt-3, 50 ng/mL of M-CSF and 25 ng/mL of GM-CSF; 0 cells/brain) were injected bilaterally with a blunt, 27-gauge needle attached to a 10 μL Model 701 RN Hamilton syringe into the lateral ventricles using the following coordinates from bregma:
i) 8 weeks old mice: AP −0.3, ML+/−1.4, DV 2.34 at a 10° angle
ii) 3 weeks old mice: AP +0.1, ML+/−1.4, DV −2.34 at a 10° angle.

### Hindlimb Clasping Test

Behavioral testing was performed at 1, 5 and/or 8 months post-surgery, depending on the cohort. Briefly, animals were removed from cage and suspended by their tail for ˜10-15 seconds and their abilities of hindlimb clasping were monitored via video recording. Hindlimb clasping was scored on a scale from 0-3 based on the following:
i) score 0: Both hindlimbs were splayed outward and away from abdomen
ii) score 1: One hindlimb is retracted toward the abdomen for more than 50% of the time the mouse is suspended from its tail
iii) score 2: Both hindlimbs are partially retracted toward the abdomen for more than 50% of the time the mouse is suspended from its tail
iv) score 3: Both hindlimbs are entirely retracted and touching the abdomen for more than 50% of the time the mouse is suspended from its tail.

### Tissue Collection and Analysis

Terminal tissue collection was performed 1-, 5-, or 8-months post myeloid cell transplantation, depending on the cohort. Briefly, animals were anaesthetized by inhalation of ˜2% isoflurane in oxygen and a small incision was made along the back of the neck to expose the muscle tissue. Next, muscle tissue was carefully removed to expose the cisterna magna and a 20 um glass pulled pipette was inserted into the cisterna magna to collect ˜3-8 ul of cerebrospinal fluid (CSF). Following CSF collection, blood was collected via intracardial puncture and then animals were perfused transcardially with cold phosphate buffered saline (PBS). Brains were dissected into two hemispheres and the left hemisphere was placed in cold 4% paraformaldehyde (PFA) for 48 h for histological analysis and the right hemisphere was flash frozen for biochemical analysis. One spinal column was collected from each group and placed in 4% PFA for 48 h for histological analysis and the remaining spinal cords were extruded via PBS flush and flash frozen for biochemical analysis.

### Histology and Immunofluorescence

Harvested tissues for histological evaluation had been fixed in 4% PFA for 48 h. Left brain hemispheres were transferred into 20% sucrose (MP Biomedicals, 0219474705) in PBS for 24 h and then into 30% sucrose in PBS for 24 h. Brains were then embedded in OCT (Sakura, 4583)/30% sucrose solution (1:1) and snap-frozen in an ethanol bath, over dry ice. 20 μm thick sections were then obtained in a sagittal orientation using a Cryostat (LEICA; CM3050 S) and mounted onto positively charged microscope slides. The slides were allowed to air dry for ˜1 h and then stored at −80° C. Spinal columns were washed in PBS and then the spinal cord was dissected from the vertebral column. Extracted spinal cords followed the same sucrose gradient submersion as described above for brain hemispheres. Following the last sucrose solution, spinal cords were dissected into cervical, thoracic, and lumbar regions, embedded in OCT solution and stored at −80° C. Spinal cords were sectioned into serial 20 μm slices on the Cryostat, mounted onto positively charged slides and stored at −80° C. For immunofluorescence staining, slides were incubated at RT for 30 m, permeabilized with 0.2% Triton-X in PBS (PBS-T), blocked for 1 h with 10% donkey serum in PBS-T and incubated with primary antibodies (Table 7 below) in blocking solution overnight at 4° C. The next day, slides were washed with PBS-T 3 times and incubated with secondary antibodies (Table 7 below) for 1 h at RT. Slides were then washed 3 times with PBS-T, incubated with 300 nM DAPI in PBS for 30 m, and mounted with Fluorsave™ Reagent (MilliporeSigma, 345789) for imaging. Slides were imaged in an Axioscan 7 microscope (Zeiss).

TABLE 7
Primary and Secondary Antibody table

| | | | | |
|---|---|---|---|---|
| Antibody | Manufacturer | Catalog # | Host | Dilution |

Primary Antibodies

| | | | | |
|---|---|---|---|---|
| Human Nuclear Antigen | Abcam | Ab191181 | Mouse | 1:50 |
| (hNA) | | | | |
| IBA1 | Fujifilm-Wako | 019-19741 | Rabbit | 1:500 |
| Ku80 | Abcam | Ab80592 | Rabbit | 1:250 |
| CD163 | R&D Systems | AF1607 | Goat | 1:200 |
| CD45 | BioLegend | 304002 | Mouse | 1:50 |
| TMEM119 | Abcam | Ab185333 | Rabbit | 1:400 |
| PU.1 | Cell Signaling | 2258S | Rabbit | 1:200 |
| Ki67 | Agilent Technologies | M724029-2 | Mouse | 1:100 |
| CD68 | Bio-Rad | MCA1957 | Rat | 1:100 |
| P2RY12 | Sigma Aldrich | HPA014518 | Rabbit | 1:250 |
| TREM2 | R&D Systems | AF1828 | Goat | 10 μg/mL |

Secondary Antibodies

| | | | | |
|---|---|---|---|---|
| Anti-Goat IgG AlexaFluor 488 | Invitrogen | A-21202 | Donkey | 1:500 |
| Anti-Mouse IgG AlexaFluor 555 | Invitrogen | A-31570 | Donkey | 1:500 |
| Anti-Rabbit IgG AlexaFluor 555 | Invitrogen | A-31572 | Donkey | 1:500 |
| Anti-Mouse IgG AlexaFluor 647 | Invitrogen | A-32787 | Donkey | 1:500 |
| Anti-Rabbit IgG AlexaFluor 647 | Invitrogen | A-31573 | Donkey | 1:500 |
| Anti-Rat IgG AlexaFluor 488 | Invitrogen | A-21208 | Donkey | 1:500 |
| Biotin-SP Anti-Rabbit IgG | Jackson | 711-065-152 | Donkey | 1:500 |
| | ImmunoResearch Laboratories | | | |
| Peroxidase Streptavidin-HRP | Jackson | 016-030-084 | | 1:250 |
| | ImmunoResearch Laboratories | | | |

### Statistical Analysis

All statistical analysis was done using Prism 9.4.1 (GraphPad Software LLC). In all graphs: *p<0.05, **p<0.01, ***p<0.001, ****p<0.0001. For hindlimb clasping, a minimum of 5 animals per group were used across all studies. As the scoring from hindlimb clasping is not normally distributed, data were analyzed using the nonparametric Kruskal-Wallis test and Conover-Iman post hoc tests were used if significant main effects were observed via Kruskal-Wallis. All data are represented as median. For enzymatic activity and GAG readouts, data was tested for normality using the Shapiro-Wilk normality test. If normally distributed, it was analyzed with a one-way ANOVA using Dunnet's multiple comparison test, comparing all groups to the vehicle group. If not normally distributed, it was analyzed with Kruskal-Wallis test followed by Dunn's multiple comparisons test. Data is represented as mean and SEM. For urine and serum samples, data was analyzed by fitting a mixed effects model followed by Dunnet's multiple comparison test, comparing all groups to the vehicle group. Data is represented as mean and SEM.

### Bulk RNA Sequencing

RNA was isolated from micro-dissected fixed histology sections using the RNeasy FFPE kit (Qiagen) and analyzed on an Agilent 2100 Bioanalyzer using Agilent 6000 RNA Nano Kit (Agilent Technologies). cDNA libraries for sequencing were generated using the Collibri 3′ mRNA Library Prep Kit for Illumina (ThermoFisher). Libraries were quantified using the Collibri Library Quantification Kit (ThermoFisher) and evaluated on the Agilent 2100 Bioanalyzer using the Agilent High Sensitivity DNA Kit (Agilent Technologies). Libraries were sequenced on the Illumina NextSeq 500 using the NextSeq 500/550 High Output Kit (75 Cycles). Reads were filtered and trimmed using Cutadapt v3.4 (Martin, M. Cutadapt removes adapter sequences from high-throughput sequencing reads. EMBnet.journal 17, 10-12 (2011) and aligned with STAR v2.7.8a (Dobin, A. et al. STAR: ultrafast universal RNA-seq aligner. Bioinforma. Oxf. Engl. 29, 15-21 (2013) to both hg38 and mm39. Aligned reads were sorted and indexed using SAMtools v1.12⁴⁴ (Li, H. et al. The Sequence Alignment/Map format and SAMtools. Bioinforma. Oxf. Engl. 25, 2078-2079 (2009) and filtered to detect only human-specific reads using XenofilteR (Kluin, R. J. C. et al. XenofilteR: computational deconvolution of mouse and human reads in tumor xenograft sequence data. BMC Bioinformatics 19, 366 (2018). Reads aligning to expressed genes were counted using HTSeq v0.13.5 (Putri, G. H., Anders, S., Pyl, P. T., Pimanda, J. E. & Zanini, F. Analysing high-throughput sequencing data in Python with HTSeq 2.0. Bioinforma. Oxf. Engl. btac166 (2022) doi:10.1093/bioinformatics/btac166).

### Single-Cell RNA Sequencing and Analysis

Fetal hematopoietic (CD45+) single-cell data from Bian et al., 2020 (Bian, Z. et al. Deciphering human macrophage development at single-cell resolution. Nature 582, 571-576 (2020) was downloaded from NCBI Gene Expression Omnibus, accession number GSE133345. For downstream analysis, primitive macrophage subpopulations (Mac_1, Mac_2, and Mac_3) were grouped together. UMAP plots were generated colored by origin, Carnegie stage and cell type grouping of all cells using Scanpy⁴⁸ (SCANPY: large-scale single-cell gene expression data analysis|Genome Biology|Full Text. https://genomebiology.biomedcentral.com/articles/10.1186/s13059-017-1382-0). Single-cell RNA-sequencing was performed on myeloid cells (n=3 independent biological replicates) and hiPSCs using 10×Genomics. The raw data for each dataset was the “Filtered Feature Barcode Matrix” output by Cell Ranger (Massively parallel digital transcriptional profiling of single cells|Nature Communications. https://www.nature.com/articles/ncomms14049) alignment and deconvolution pipeline. Each dataset was imported and mitochondrial content, number of genes by counts, and total counts annotated for each cell. A knee locator-based method (Satopaa, V., Albrecht, J., Irwin, D. & Raghavan, B. Finding a ‘Kneedle’ in a Haystack: Detecting Knee Points in System Behavior. (2010) was used to remove cells that have an overrepresentation of mitochondrial counts. The data was then filtered to include only cells that have a minimum of 1000 non-zero transcripts. Double detection and removal were performed and the dataset subset to only include 5000 cells (so that each dataset has the same number of cells in the analysis). Each dataset was iteratively joined to the other datasets included in the analysis. Joining is performed on Ensembl IDs on an inner-join basis (only genes common to both datasets are included in the resulting data object). Myeloid cell and hiPSC data was then joined to Bien at al. data on Ensembl IDs. Finally, the total counts per cell was normalized to 1,000,000 (CPM normalization), and log 2 transformed. PCA was run on the concatenated dataset using highly variable genes. Differential gene expression was run using Scanpy's rank_genes_groups function and filtered based on a minimum log fold change=4 and a threshold fraction of cells expressing the gene within and outside of the group=0.5. Genes enriched in the Mac 1-3 groups and Mac 4 group were used for sparsity-based scoring. To score, the normalized expression matrix was binarized and the sparsity of the matrix was calculated. The matrix was subsetted to include the genes in the group of interest (Mac 1-3 or Mac 4). The gene set score for each cell was calculated as the sum of the binarized expression data for the gene group multiplied by the sparsity, normalized by the maximum score so that the value ranged from 0 to 1. Differential gene expression was run on the Bian et al. dataset (with the same settings described previously) based on the cell type groupings of all cells to find unique markers for each group. Expression data from this dataset was standardized between 0 and 1 and shown as a heatmap. Unique genes found in the Mac 4 group and Mac 1-3 groups were displayed as dotplots using the single-cell RNA sequencing datasets and as a heatmap using engrafted myeloid cell bulk RNAseq expression data.

### Miscellaneous Software

Figures were created and assembled in Affinity Designer 1.10. Varied illustrations were created with BioRender.com.

1. A method of treating a metabolic disorder comprising:
administering myeloid cells into a central nervous system of a subject to be treated;
allowing the administered myeloid cells to engraft and to produce an enzyme;
monitoring the level of the enzyme in a serum sample, a urine sample or a cerebrospinal fluid sample of the subject to be treated; and
monitoring the amount of a substrate in the serum sample, a urine sample or a cerebrospinal fluid sample of the subject to determine the progress of the treatment.

2. The method of claim 1, wherein said myeloid cells are pluripotent stem cell (PSC)-derived myeloid cells.

3. The method of claim 1, wherein said myeloid cells are non-pluripotent stem cell (PSC)-derived myeloid cells.

4. The method of claim 3, wherein said non-pluripotent stem cell (PSC)-derived myeloid cells are peripheral blood mononuclear cells (PBMC).

5. The method of claim 1, wherein said metabolic disorder is an inherited metabolic disorder.

6. The method of claim 2, wherein said inherited metabolic disorder is Hurler Syndrome.

7. The method of claim 3, wherein said enzyme is alpha L-iduronidase (IDUA).

8. The method of claim 2, wherein said inherited metabolic disorder is Sly Syndrome.

9. The method of claim 5, wherein said enzyme is Beta-glucuronidase (GUSB).

10. The method of claim 1, wherein said central nervous system is selected from the group consisting of intracerebroventricular (ICV) space, brain, spinal cord, cerebrospinal fluid (CSF), and intraparenchymal space.

11. The method of claim 1, wherein said subject is a mouse or human.

12. The method of claim 1, wherein said substrate is glycosaminoglycans.

13. The method of claim 1, wherein said subject to be treated has an accumulation of undegraded substrate in cells of said subject.

14. The method of claim 1, wherein said enzyme is used to reduce the accumulated undegraded substrate in brain cells of the subject to be treated.

15. The method of claim 1, wherein a sustained reduction in total substrate levels greater than about 20% is indicative of successful treatment of said metabolic disorder.

16. The method of claim 1 wherein the amount of enzyme in said serum sample is compared to the amount of enzyme in said serum sample of a healthy subject.

17. The method of claim 1 wherein the activity of enzyme in said serum sample is compared to the activity of enzyme in said serum of a healthy subject.

18. The method of claim 1, wherein the amount of said cells to be injected in a human is in the range of about 25×10⁶ to about 1250×10⁶ cells.

19. (canceled)

20. The method of claim 1, wherein the amount of said cells to be injected in a human is in the range of about 100×10⁶ to about 500×10⁶ cells.

21.-22. (canceled)

23. The method of claim 13, wherein said cells are brain cells.

24. The method of claim 23, wherein said brain cells are selected from the group consisting of neurons, oligodendrocytes, astrocytes, microglia, perivascular macrophages, meningial macrophages, endothelial cells, pericytes, ependymal cells and blood cells.

25. The method of claim 5, wherein said inherited metabolic disorder is a lysosomal storage disease.

26. The method of claim 25, wherein said lysosomal storage diseases are selected from the group consisting of MPS I (Hurler Syndrome), MPS IIIA (Sanfilippo A), MPS IIIB (Sanfilippo B), MPS VII (Sly Syndrome), Krabbe disease, Pompe (glycogen storage disease type II), GM1-gangliosidosis, GM2-gangliosidosis (Sandhoff/Tay-Sachs) and Metachromatic leukodystrophy.

27.-34. (canceled)


SEQUENCE LISTING (12 sequences)

SEQ ID NO: 1 - DNA, 20 bp, synthetic construct
  misc_feature 1..20: note = IDUA sgRNA
  source 1..20: mol_type = other DNA
gttccagggtttgagctgat

SEQ ID NO: 2 - DNA, 20 bp, synthetic construct
  misc_feature 1..20: note = GUSB sgRNA
  source 1..20: mol_type = other DNA
atcgatgagtgtcccggcgt

SEQ ID NO: 3 - DNA, 20 bp, synthetic construct
  misc_feature 1..20: note = NAGLU sgRNA
  source 1..20: mol_type = other DNA
ggaacatgccccgcgaatgc

SEQ ID NO: 4 - DNA, 20 bp, synthetic construct
  misc_feature 1..20: note = SGSH sgRNA
  source 1..20: mol_type = other DNA
cctcacgcagcggatgacgg

SEQ ID NO: 5 - DNA, 19 bp, synthetic construct
  misc_feature 1..19: note = IDUA Forward Primer
  source 1..19: mol_type = other DNA
gacgctgaccgtccttctg

SEQ ID NO: 6 - DNA, 18 bp, synthetic construct
  misc_feature 1..18: note = IDUA Reverse Primer
  source 1..18: mol_type = other DNA
cgcctgcccaccaatgta

SEQ ID NO: 7 - DNA, 20 bp, synthetic construct
  misc_feature 1..20: note = GUSB Forward Primer
  source 1..20: mol_type = other DNA
tccacggtgtcaacaagcat

SEQ ID NO: 8 - DNA, 20 bp, synthetic construct
  misc_feature 1..20: note = GUSB Reverse Primer
  source 1..20: mol_type = other DNA
gggtggttcttgtccctacg

SEQ ID NO: 9 - DNA, 19 bp, synthetic construct
  misc_feature 1..19: note = NAGLU Forward Primer
  source 1..19: mol_type = other DNA
aacctgcacacctgggatg

SEQ ID NO: 10 - DNA, 23 bp, synthetic construct
  misc_feature 1..23: note = NAGLU Reverse Primer
  source 1..23: mol_type = other DNA
tgtgcaaagtcacagatagatcg

SEQ ID NO: 11 - DNA, 20 bp, synthetic construct
  misc_feature 1..20: note = SGSH Forward Primer
  source 1..20: mol_type = other DNA
gatgtacccactcgggtgtc

SEQ ID NO: 12 - DNA, 20 bp, synthetic construct
  misc_feature 1..20: note = SGSH Reverse Primer
  source 1..20: mol_type = other DNA
tctgcactcacagccatagg